(19) United States
(12) Patent Application Publication
(10) Pub. No.: US 2014/0178461 A1
(43) Pub. Date: Jun. 26, 2014

(54) COMPOUNDS AND COMPOSITIONS FOR USE IN THE TREATMENT AND PREVENTION OF LUNG AND BRAIN CANCER AND PRECANCEROUS CONDITIONS THEREOF

(71) Applicant: Medicon Pharmaceuticals, Inc., Setauket, NY (US)

(72) Inventor: Basil RIGAS, Setauket, NY (US)

(21) Appl. No.: 14/033,932

(22) Filed: Sep. 23, 2013

Related U.S. Application Data
(60) Provisional application No. 61/703,980, filed on Sep. 21, 2012.

(51) Int. Cl. A61K 31/675; A61K 45/06; C07F 9/572; A61K 31/661; C07F 9/09

(52) U.S. Cl. CPC A61K 31/675; A61K 31/661; C07F 9/09; C07F 9/5728; A61K 45/06
USPC 424/450; 514/119; 558/170; 514/80; 548/414; 131/290

(57) ABSTRACT

Novel compounds and pharmaceutical compositions thereof administered by the respiratory route for prevention and/or treatment of lung and brain cancer and precancerous conditions thereof.

### FIELD OF THE INVENTION

The invention is directed to compounds and pharmaceutical compositions for the prevention and/or treatment of lung and brain cancer and precancerous conditions thereof.

### BACKGROUND OF THE INVENTION

Lung cancer is the major cause of cancer mortality in the industrial world. Despite significant advances in its early detection, the survival of lung cancer patients remains poor, with the 5-year survival being as low as 5%. Because of frequent and widespread metastases, surgical procedures for lung cancer are not particularly effective and therefore chemotherapy often is the treatment of choice. The efficacy of chemotherapy against lung cancer is, however, limited primarily by the intrinsically low anticancer activity of available agents; the development of drug resistance; and drug toxicity. Therefore, there is a pressing need for the development not only of new drugs but also of methods of their administration to treat lung cancer and its precancerous conditions.

An important approach to the control of lung cancer is the form of cancer prevention known as chemoprevention, i.e., the administration of natural or synthetic agents to subjects at risk of cancer to prevent its development or its recurrence in those who already had a cancer. When effective, chemoprevention abrogates the development of lung cancer. Prominent among those individuals at risk of lung cancer are former and current smokers, and those with its precancerous conditions. The opportunity for the chemoprevention of lung cancer is provided by the fact that the development of lung cancer represents a long transition of the tracheal epithelium from normal through various precancerous stages to lung cancer. Therefore, chemoprevention (administered during this transitional period) is a simpler and more cost-effective approach compared to treating an already developed lung cancer.

Regarding the treatment and/or prevention of lung cancer and its precancerous conditions, there is a need for a) novel anticancer drugs and b) improved methods to administer such drugs, which would enhance their delivery to the lung and limit systemic drug exposure, thus reducing side effects.

Several components of tobacco smoke are carcinogenic and smoking has also been recently linked to increased risk of brain cancers such as glioma. Furthermore, primary lung cancers can spread to the brain with 40% of patients with lung cancers developing brain metastases; small cell lung cancer can spread to the brain even before it is diagnosed. Therefore, there is a need for new methods to prevent and/or treat brain cancers and their precancerous conditions in persons at increased risk of developing lung or brain cancers, in particular smokers.

Even though it is widely known that smoking causes lung cancer, smoking is a very difficult addiction to break and currently-marketed smoking cessation products are of limited efficacy. Even patients diagnosed with lung or brain cancer or with precancerous conditions thereof often fail to quit smoking.

Accordingly, there is a need for new methods of therapy and prevention of lung and brain cancer and precancerous conditions thereof for smokers.

WO 2009/023631 discloses compounds suitable for the treatment of cancer.

The inventor surprisingly found that some derivatives, in particular phospho-derivatives of certain compounds can be employed for the treatment and/or prevention of lung and brain cancer and precancerous conditions thereof, when these compounds are administered by the respiratory route i.e. by the nasal or oral respiratory route. This administration can be combined with smoking and/or consuming a smoking-cessation product, and, therefore, is particularly suitable for smokers.

### SUMMARY OF THE INVENTION

The present invention provides a novel therapy and prevention and/or treatment of lung and brain cancer and precancerous conditions thereof.

The invention provides compounds of general Formula I and compositions thereof and their use in the treatment and/or prevention of lung and brain cancer and precancerous conditions thereof, wherein said compounds are administered to a human or animal by the respiratory route including the nasal passages. In a preferred embodiment, the invention provides the use of certain derivatives of non-steroidal anti-inflammatory drugs (NSAIDs) in the treatment and/or prevention of lung and brain cancer and precancerous conditions thereof, wherein said NSAID derivatives are administered to a human or animal by the respiratory route.

The present invention relates to a compound of general Formula I

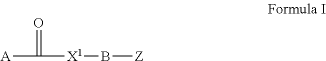

or an enantiomer, a diastereomer, a racemate, a tautomer, salt or hydrate or cocrystal thereof, for use in the treatment and/or prevention of lung and brain cancer and precancerous conditions thereof, wherein said treatment and/or prevention comprises administering a pharmaceutically effective dose of the compound to a human or animal in need thereof.
X¹ is selected from the group consisting of —O—, —S— and —NR¹—,
R¹ being hydrogen or C₁₋₁₀₀-alkyl, preferably C₁₋₂₂-alkyl, particularly preferred C₁₋₁₀-alkyl.
A is an optionally substituted aliphatic, heteroaliphatic, aromatic, heteroaromatic substituent or alkylaryl substituent having in a preferred embodiment 1 to 100, and even more preferably 1 to 42 carbon atoms. Preferably, A is derived from among NSAIDs. In one of the preferred embodiments, A is selected from the group consisting of

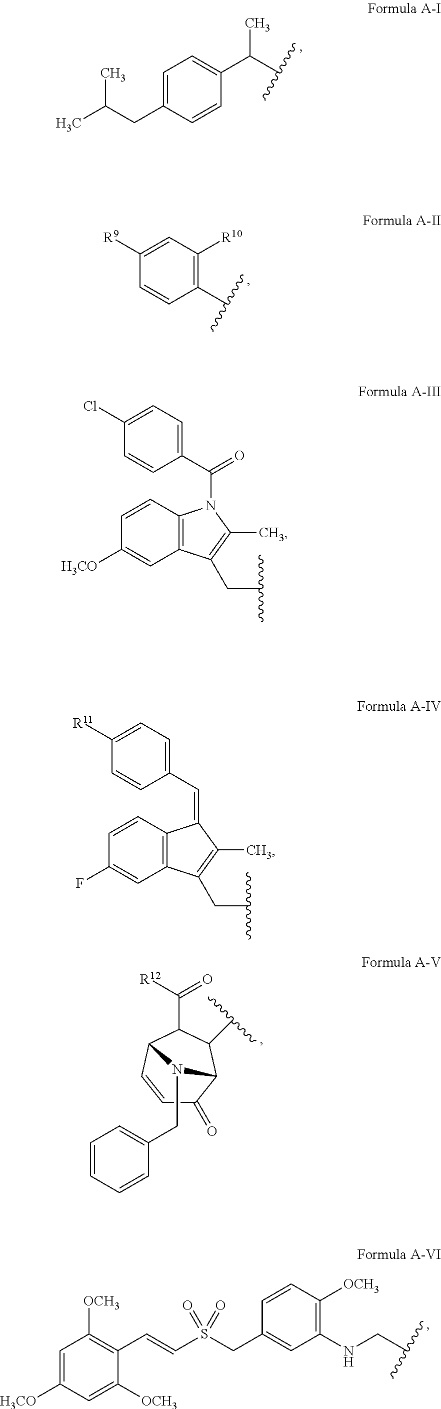

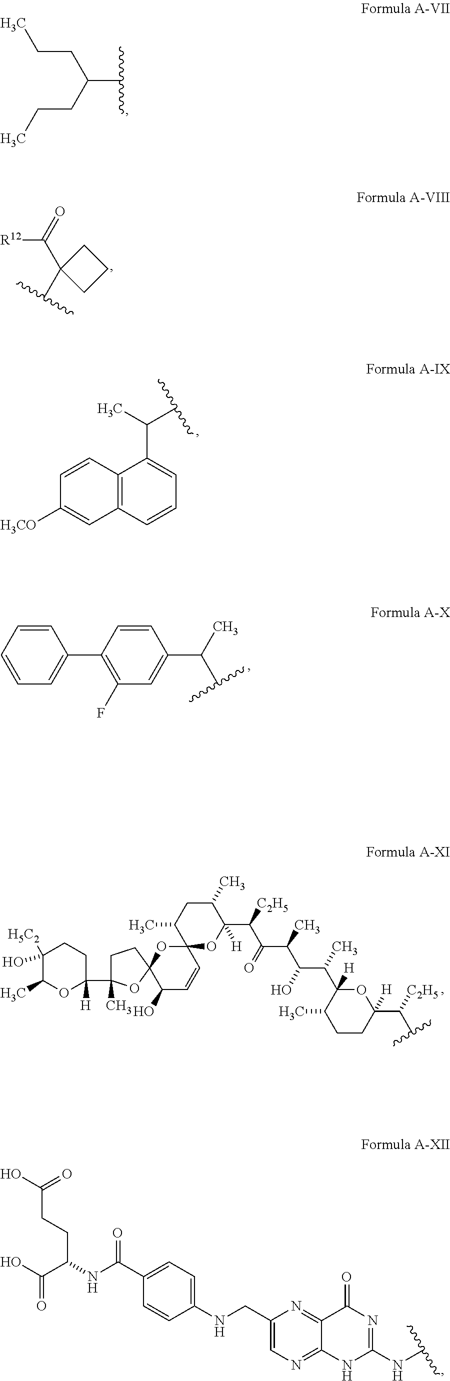

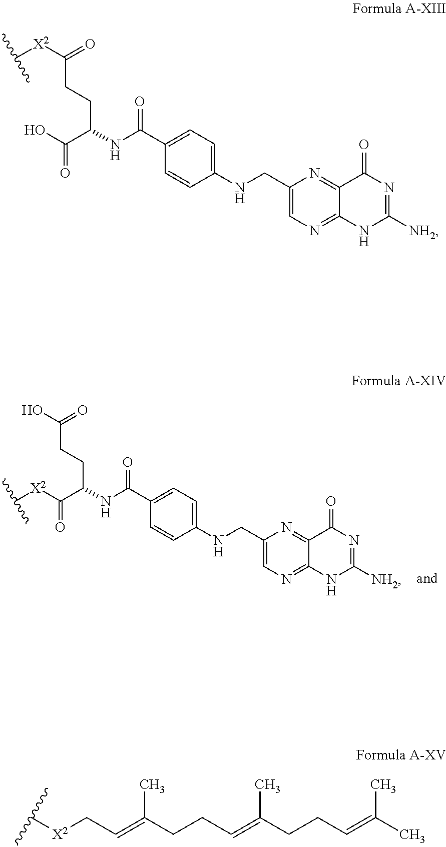

R⁹ being selected from hydrogen and trifluoromethyl;
R¹⁰ being selected from —X²—C(O)—CH₃,
R¹¹ being selected from —SCH₃, —S(O)CH₃ and —S(O)₂CH₃;
R¹² being selected from hydroxy, —B—Z and Formula A-XII
   whereby X² is selected from the group consisting of —O—, —S— and —NR¹³—,
   R¹³ being hydrogen or C₁₋₆-alkyl.
   B is selected from the group consisting of

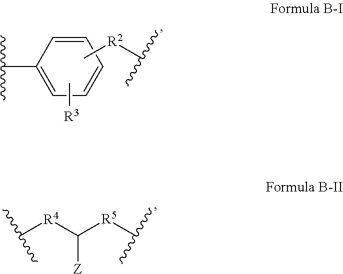

a single bond and an aliphatic substituent, preferably with 1 to 100, more preferred with 1 to 42 and particularly preferred with 1 to 22 carbon atoms,
R², R⁴ and R⁵ being the same or different C₁₋₃-alkylene,
R³ being hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)O—C₁₋₆-alkyl, —OC(O)—C₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxyl.
Z is selected independently from the group consisting of

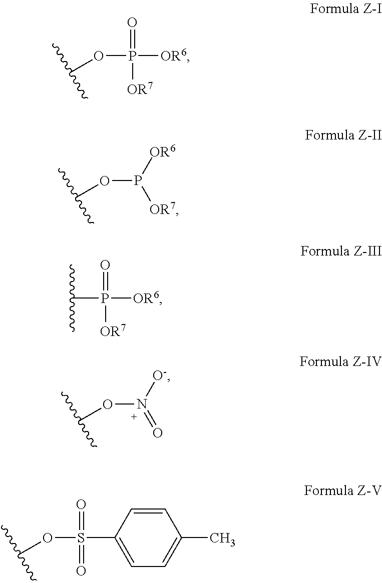

and a folic acid residue;
R⁶ being independently selected from hydrogen, C₁₋₁₀₀-alkyl, preferably C₁₋₆-alkyl, and polyethylene glycol residue,
R⁷ being selected from hydrogen, C₁₋₁₀₀-alkyl, preferably C₁₋₆-alkyl, and polyethylene glycol residue;
or B together with Z forms a structure

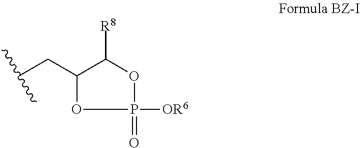

R⁶ being defined as above,
R⁸ being independently selected from hydrogen, an aliphatic substituent, preferably with 1 to 22 carbon atoms, more preferred C₁₋₆-alkyl, and a polyethylene glycol residue.

Preferably, the folic acid residue is selected from the group consisting of

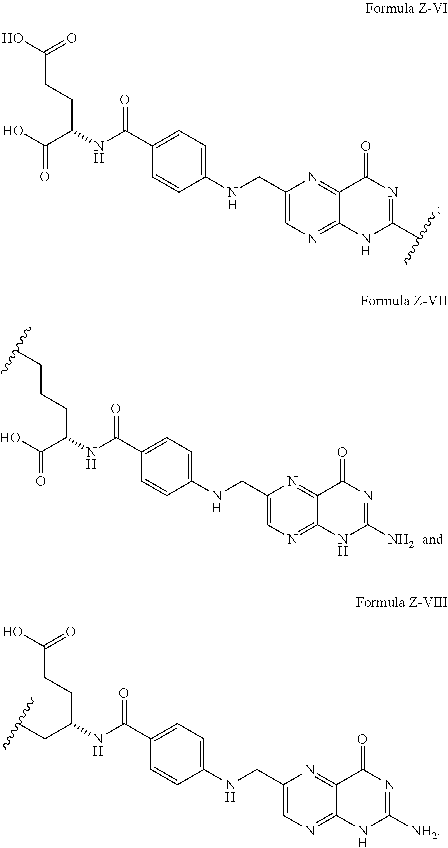 [SMILES: *c1nc(=O)c2nc(CNc3ccc(C(=O)N[C@@H](CCC(=O)O)C(=O)O)cc3)cnc2[nH]1 ; *CCC[C@H](NC(=O)c1ccc(NCc2cnc3[nH]c(N)nc(=O)c3n2)cc1)C(=O)O ; *C[C@H](CCC(=O)O)NC(=O)c1ccc(NCc2cnc3[nH]c(N)nc(=O)c3n2)cc1]

In one embodiment, A is represented by Formula A-I or A-IV, X¹ is —O— and —B—Z is not —(CH₂)₄—O—P(O)(OC₂H₅)₂.

In another embodiment, A is represented by Formula A-II and X′ is not —O— and/or —B— is an aliphatic substituent with 1 to 100, preferably with 1 to 42 carbon atoms.

In a further embodiment of the present invention, the compound of Formula I is used to prevent or to treat lung cancer, the lung cancer being preferably selected from the group consisting of small cell lung cancer and non-small cell lung cancer.

In a further embodiment, the compound of Formula I is used to prevent a precancerous condition of the brain such as a precancerous brain lesion.

In still a further embodiment, the compound of Formula I is used to prevent or to treat brain cancer, for instance a glioma.

A further embodiment of the present invention relates to prevention of a precancerous condition of the lung such as a precancerous lung lesion.

Another aspect of the present invention relates to a pharmaceutical composition comprising the compound of the present invention for use in the treatment and prevention of lung and brain cancer and precancerous conditions thereof, wherein said composition is administered to a human or animal by the respiratory route.

The pharmaceutical composition of the present invention may, for instance, be administered to a human or animal by nasal administration.

In some embodiments, the present invention relates to the pharmaceutical composition of the present invention, wherein said composition is administered to a human or animal in the form of an aerosol.

In a further embodiment, the pharmaceutical composition of the present invention is administered to a human or animal in the form of a dry powder aerosol.

The pharmaceutical composition of the present invention can be formulated in the form of nanoparticles. Said nanoparticles may be lipid or polymeric nanoparticles or combinations thereof. Said nanoparticles may also be in form of a liposome, submicron emulsion, microemulsion, nanoemulsion, lipid micelle, solid lipid nanoparticle, polymeric micelle, polymeric nanoparticle or combinations thereof.

The pharmaceutical composition of the present invention may be formulated with another additional compound having anticancer activity, for instance, with difluoromethylornithine or with tyrosine kinase inhibitors such as erlotinib or with compounds enhancing oxidative stress such as thiostrepton.

In yet another embodiment of the present invention, the pharmaceutical composition is administered to a human or animal, preferably to a human, in combination with tobacco smoke.

In a further aspect, the present invention is directed to an inhalation device comprising the pharmaceutical composition of the present invention.

Yet another aspect of the present invention is directed to a smoking device, for instance, to a cigarette, comprising tobacco and the pharmaceutical composition of the present invention.

In a particularly preferred embodiment of such a smoking device of the present invention, the pharmaceutical composition is spatially separated from the tobacco.

A further aspect of the invention relates to a method of treating and/or preventing lung cancer and precancerous conditions of the lung, wherein said method comprises administering to a human or animal in need thereof, a pharmaceutically effective amount of the compound of Formula I, or of the pharmaceutical composition thereof, wherein said administration is by the respiratory route.

Such administration is effected via the inhalation device, or via the smoking device described in the application.

Still a further aspect of the invention relates to a product comprising a nicotine-containing material and an anti-cancer agent, wherein the anti-cancer agent preferably comprises the compound of Formula I.

In some embodiments, the anti-cancer agent may be an oxidative stress enhancer.

The anti-cancer agent may also comprise a combination of at least two different compounds having anti-cancer activity, preferably a combination of curcumin and of the compound of Formula I.

In one preferred embodiment, the nicotine-containing material is tobacco leaf.

Preferably, the product of the present invention contains nicotine and the anti-cancer agent in the ratio of from 1000:1 to 1:10 (wt:wt).

In some embodiments, the product is a smoking device selected from the group consisting of cigarette, cigar and smoking pipe, the smoking device optionally including an additional unit which renders the anti-cancer agent suitable for inhalation.

In other embodiments, the product is a smoking cessation product.

In some embodiments of the invention, the product is a transdermal patch.

In some further embodiments of the invention, the product is an inhalation device.

In some further embodiments of the invention, the product is an electronic cigarette.

In some further embodiments of the invention, the product is an orally applied product, for instance a smokeless tobacco product.

A further aspect of the invention relates to an anti-cancer agent for use in the prevention and/or treatment of cancer and/or precancerous conditions, wherein said anti-cancer agent is administered simultaneously with nicotine. The cancer may be, for instance, a lung cancer, brain cancer or a precancerous condition thereof.

In one preferred embodiment, the anti-cancer agent is inhaled together with tobacco smoke.

### BRIEF DESCRIPTION OF THE FIGURES

FIG. 1. Nose-only aerosol exposure system.

FIGS. 2A-2F. Modes of administration of the compound of Formula I.

FIG. 3. Biodistribution of liposomal phospho-ibuprofen amide 1 in mice after i.v. administration at 200 mg/kg.

FIG. 4. Inhibition of human lung cancer by phospho-ibuprofen amide 1.

FIG. 5. Inhibition of human lung cancer by phospho-ibuprofen amide 1.

FIG. 6. Pharmacokinetic study of PTI 93 in mice.

FIG. 7. Effective inhibition of human cancer cell xenograft tumor growth by PTI 93.

FIG. 8. Levels of phospho-sulindac (PS, 96) and its metabolites in the lungs (A) and plasma (B) of mice subjected to aerosol administration of PS 96.

FIG. 9. Survival rates of control and aerosolized-PS treated groups of mice implanted orthotopically with A549 cells.

FIG. 10. Aerosol administration of PS 96.

FIG. 11. Aerosol administration of PS 96.

FIG. 12. Lung level of PS 96 after inhalation and oral administration.

FIG. 13. Plasma level of PS 96 after inhalation and oral administration.

FIG. 14. Phosphovalproic acid (PV, 116) and ibuprofen phospho-glycerol amide (PGIA, 4) synergize strongly to inhibit the growth of glioblastoma and lung cancer

### DETAILED DESCRIPTION OF THE INVENTION

The term “aliphatic substituent”, as used herein, includes saturated or unsaturated, branched or unbranched aliphatic univalent or bivalent substituents. In the present application, aliphatic substituent is intended to include, but is not limited to, alkyl, cycloalkyl, alkylene, alkenylene, alkynylene and alkadienylene substituents. According to the present invention, the aliphatic substituent has 1 to 100, preferably 1 to 42 carbon atoms, preferably 1 to 22 carbon atoms, more preferred 1 to 15 carbon atoms, further preferred 1 to 10 carbon atoms, even more preferred 1 to 6 carbon atoms, for instance 4 carbon atoms. Most preferably, the aliphatic substituent is C₁₋₆-alkylene, e.g. methylene, ethylene, trimethylene and tetramethylene.

The term “alkyl” used is the present application relates a saturated branched or unbranched aliphatic univalent substituent. Preferably, the alkyl substituent has 1 to 100 carbon atoms, more preferred 1 to 22 carbon atoms, further preferred 1 to 10 carbon atoms, yet more preferred 1 to 6 carbon atoms, even more preferred 1 to 3 carbon atoms. Accordingly, examples of the alkyl substituent include methyl, ethyl, n-propyl, isopropyl, n-butyl, isobutyl, sec-butyl, tert-butyl, n-pentyl and n-hexyl and preferable examples include methyl, ethyl, n-propyl and isopropyl, whereby ethyl and isopropyl are particularly preferred.

As used herein, the term “cycloalkyl” refers to a monocyclic, bicyclic, or tricyclic substituent, which may be saturated or partially saturated, i.e. possesses one or more double bonds. Monocyclic substituents are exemplified by a saturated cyclic hydrocarbon group containing from 3 to 8 carbon atoms. Examples of monocyclic cycloalkyl substituents include cyclopropyl, cyclobutyl, cyclopentyl, cyclopentenyl, cyclohexyl, cyclohexenyl, cycloheptyl and cyclooctyl. Bicyclic fused cycloalkyl substituents are exemplified by a cycloalkyl ring fused to another cycloalkyl ring. Examples of bicyclic cycloalkyl substituents include, but are not limited to decalin, 1,2,3,7,8,8a-hexahydro-naphthalene, and the like. Tricyclic cycloalkyl substituents are exemplified by a cycloalkyl bicyclic fused ring fused to an additional cycloalkyl substituent.

The term “alkylene” used is the present application relates a saturated branched or unbranched aliphatic bivalent substituent. Preferably, the alkylene substituent has 1 to 6 carbon atoms, more preferred 1 to 3 carbon atoms. Accordingly, examples of the alkylene substituent include methylene, ethylene, trimethylene, propylene, tetramethylene, isopropylidene, pentamethylene and hexamethylene. Preferable examples of the alkylene substituent include methylene, ethylene, trimethylene and tetramethylene, whereby tetramethylene is particularly preferred.

The term “alkenylene” as used is the present application is an unsaturated branched or unbranched aliphatic bivalent substituent having a double bond between two adjacent carbon atoms. Preferably, the alkenylene substituent has 2 to 6 carbon atoms, more preferred 2 to 4 carbon atoms. Accordingly, examples of the alkenylene substituent include but are not limited to vinylene, 1-propenylene, 2-propenylene, methylvinylene, 1-butenylene, 2-butenylene, 3-butenylene, 2-methyl-1-propenylene, 2-methyl-2-propenylene, 2-pentenylene, 2-hexenylene. Preferable examples of the alkenylene substituent include vinylene, 1-propenylene and 2-propenylene, whereby vinylene is particularly preferred.

The term “alkynylene” as used is the present application is an unsaturated branched or unbranched aliphatic bivalent substituent having a tripple bond between two adjacent carbon atoms. Preferably, the alkynylene substituent has 2 to 6 carbon atoms, more preferred 2 to 4 carbon atoms. Examples of the alkynylene substituent include but are not limited to ethynylene, 1-propynylene, 1-butynylene, 2-butynylene, 1-pentynylene, 2-pentynylene, 3-pentynylene and 2-hexynylene. Preferable examples of the alkenylene substituent include ethynylene, 1-propynylene and 2-propynylene, whereby ethynylene is particularly preferred.

The term “alkadienylene” as used is the present application is an unsaturated branched or unbranched aliphatic bivalent substituent having two double bonds between two adjacent carbon atoms. Preferably, the alkadienylene substituent has 4 to 10 carbon atoms. Accordingly, examples of the alkadienylene substituent include but are not limited to 2,4-pentadienylene, 2,4-hexadienylene, 4-methyl-2,4-pentadienylene, 2,4-heptadienylene, 2,6-heptadienylene, 3-methyl-2,4-hexadienylene, 2,6-octadienylene, 3-methyl-2,6-heptadienylene, 2-methyl-2,4-heptadienylene, 2,8-nonadienylene, 3-methyl-2,6-octadienylene, 2,6-decadienylene, 2,9-decadienylene and 3,7-dimethyl-2,6-octadienylene substituents, whereby 2,4-pentadienylene is particularly preferred.

The term “heteroaliphatic substituent”, as used herein, refers to a monovalent or a bivalent substituent, in which one or more carbon atoms have been substituted with a heteroatom, for instance, with an oxygen, sulfur, nitrogen, phosphorus or silicon atom, wherein the nitrogen and sulfur atoms may optionally be oxidized, and the nitrogen heteroatom may optionally be quaternized. The heteroatom(s) O, N and S may be placed at any interior position of the heteroaliphatic substituent. Examples include —CH₂—CH₂—O—CH₃, —CH₂—CH₂—NH—CH₃, —CH₂—CH₂—N(CH₃)—CH₃, —CH₂—S—CH₂—CH₃, —S(O)—CH₃, —CH₂—CH₂—S(O)₂—CH₃, —CH═CH—O—CH₃, —CH₂—CH═N—OCH₃, and —CH═CH—N(CH₃)—CH₃. A heteroaliphatic substituent may be linear or branched, and saturated or unsaturated.

In one preferred embodiment, the heteroaliphatic substituent has 1 to 100, preferably 1 to 42 carbon atoms. In yet another preferred embodiment, the heteroaliphatic substituent is a polyethylene glycol residue.

The term “polyethylene glycol” (PEG) refers to a compound of formula H—(OCH₂CH₂)ₙOH in which n has a value typically from 21 to 135, but not restricted to this range. Commercial polyethylene glycols having number average molecular weights of 1,000, 1,500, 1,540, 4,000 and 6,000 are useful in this invention. These solid polyethylene glycols have melting points of 35° C. to 62° C. and boiling or flash points ranging from 430° C. to over 475° C. The preferred polyethylene glycol residues falling within the definition of the present invention are those having the formula —(OCH₂CH₂)ₙOCH₃ in which n is from 21 through 135, and preferably from 40 to 50.

As used herein, “aromatic substituent” is intended to mean any stable monocyclic, bicyclic or polycyclic carbon ring of up to 10 atoms in each ring, wherein at least one ring is aromatic, and may be unsubstituted or substituted. Examples of such aromatic substituents include phenyl, p-toluenyl (4-methylphenyl), naphthyl, tetrahydronaphthyl, indanyl, biphenyl, phenanthryl, anthryl or acenaphthyl. In cases where the aromatic substituent is bicyclic and one ring is non-aromatic, it is understood that attachment is via the aromatic ring.

The term “arylalkyl substituents” refers to alkyl substituents as described above wherein one or more bonds to hydrogen contained therein are replaced by a bond to an aryl substituent as described above. It is understood that an arylalkyl substituents is connected to the carbonyl group if the compound of Formula I through a bond from the alkyl substituent. Examples of arylalkyl substituents include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl and the like.

The term “heteroaromatic substituent” as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic heteroaromatic substituents include phenyl, pyridine, pyrimidine or pyridizine rings that are
a) fused to a 6-membered aromatic (unsaturated) heterocyclic ring having one nitrogen atom;
b) fused to a 5- or 6-membered aromatic (unsaturated) heterocyclic ring having two nitrogen atoms;
c) fused to a 5-membered aromatic (unsaturated) heterocyclic ring having one nitrogen atom together with either one oxygen or one sulfur atom; or
d) fused to a 5-membered aromatic (unsaturated) heterocyclic ring having one heteroatom selected from O, N or S.

Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The aliphatic, heteroaliphatic, aromatic and heteroaromatic substituents can be optionally substituted one or more times, the same way or differently with any one or more of the following substituents including, but not limited to: aliphatic, heteroaliphatic, aromatic and heteroaromatic substituents, aryl, heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃: —CH₂CF₃; —CHCl₂; —CH₂OH; —CH₂CH₂OH; —CH₂NH₂; —CH₂SO₂CH₃; —C(O)Rₓ, —CO₂(Rₓ); —CON(Rₓ)₂; —OC(O)Rₓ; —OCO₂Rₓ; —OCON(Rₓ)₂; —N(R_{X})₂; —S(O)Rₓ; —S(O)₂Rₓ; —NRₓ(CO)Rₓ wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, heteroaryl, (alkyl)aryl or (alkyl)heteroaryl substituents described above and herein may be substituted or unsubstituted. Additionally, it will be appreciated, that any two adjacent substituents taken together may represent a 4, 5, 6, or 7-membered substituted or unsubstituted alicyclic or heterocyclic substituents. Additional examples of generally applicable substituents are illustrated by the specific embodiments shown below.

The terms “halo” and “halogen” refer to a halogen atom selected from the group consisting of F, Cl, Br and I. Preferably the halogen atom is ClI or Br, whereby CI is particularly preferred.

The term “halogenated alkyl substituent” refers to an alkyl substituents as defined above which is substituted with at least one halogen atom. In a preferred embodiment, the halogenated alkyl substituent is perhalogenated. In a more preferred embodiment, the halogenated alkyl substituent is a univalent perfluorated substituent of formula CₙF₂ₙ₊₁. Preferably, the halogenated alkyl substituent has 1 to 6 carbon atoms, even more preferred 1 to 3 carbon atoms. Accordingly, examples of the alkyl group include trifluoromethyl, 2,2,2-trifluoroethyl, n-perfluoropropyl, n-perfluorobutyl and n-perfluoropentyl. Preferable examples of halogenated alkyl substituents include trifluoromethyl and 2,2,2-trifluoroethyl, whereby trifluoromethyl is particularly preferred.

The term “smoking” as used herein, refers to the action of inhaling or tasting the smoke of burning plant material, preferably of tobacco leaves. Smoking further includes a process wherein the smoking composition is heated but not pyrolysed, and the heated vapors are inhaled or tasted by the smoker.

One aspect of the present invention relates to the compound of Formula I:

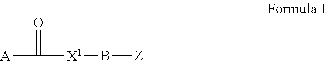

or an enantiomer, a diastereomer, a racemate, a tautomer, salt or hydrate or cocrystal thereof.

A is an optionally substituted aliphatic, heteroaliphatic, aromatic, heteroaromatic substituent or alkylaryl substituent having 1 to 100, preferably 1 to 42 carbon atoms. Preferably, A is derived from among non-steroidal anti-inflammatory drugs (NSAIDs) having a carboxylic acid moiety in the structure, whereby the carbonyl group of said carboxylic acid moiety corresponds to the carbonyl group in the Formula I.

In some particularly preferred embodiments, the substituent A is derived from among the NSAIDs ibuprofen (Formula A-I), Aspirin® (Formula A-II), indomethacin (Formula A-III) or sulindac (Formula A-IV):

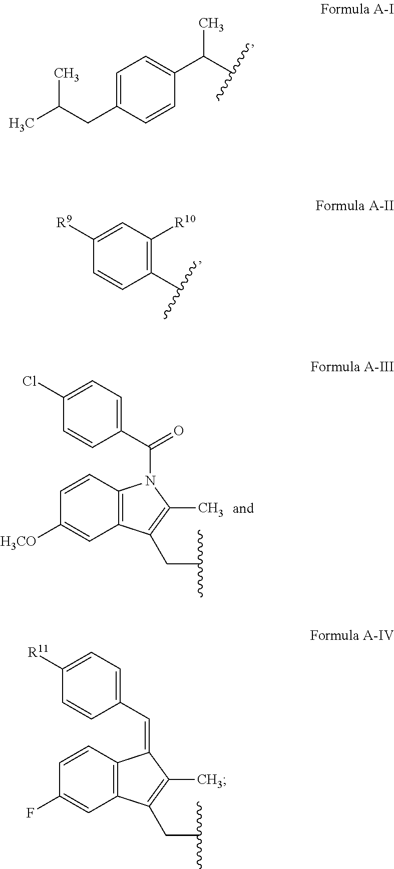

R⁹ being selected from hydrogen and trifluoromethyl,
R¹⁰ being selected from selected from —X²—C(O)—CH₃,

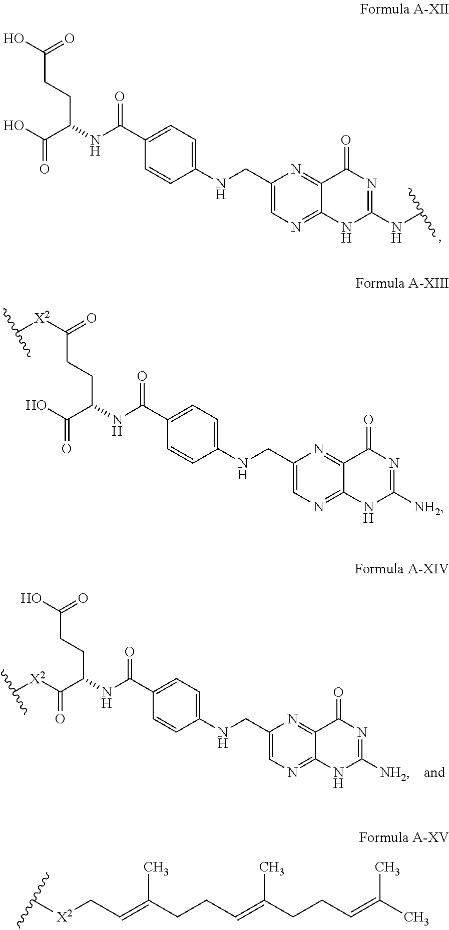

R¹¹ being selected from —SCH₃, —S(O)CH₃, —S(O)₂CH₃ and
X² being selected from the group consisting of —O—, —S— and —NR¹³—, whereby R¹³ is hydrogen or C₁₋₆-alkyl.

The substituent R¹¹ in Formula A-IV is selected from the group consisting of —SCH₃, —S(O)CH₃, —S(O)₂CH₃. Preferably R¹¹ in Formula A-IV is —S(O)CH₃.

In some other embodiments the substituent A is represented by Formulae A-V to A-XI shown below:

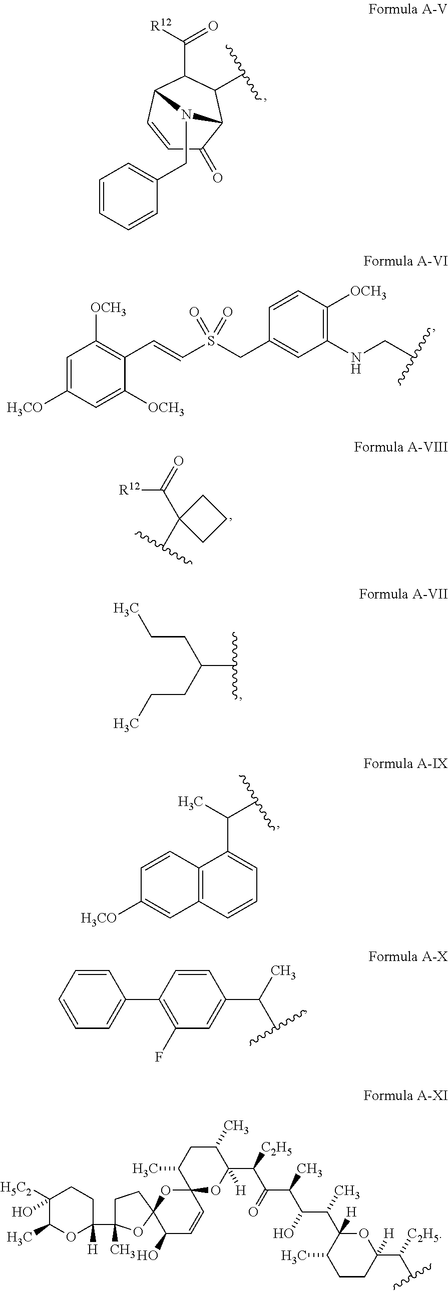

The substituent X¹ in Formula I can be —O—, —S— or —NR¹—, R¹ being hydrogen or an alkyl group having 1 to 100, preferably 1 to 22 carbon atoms, more preferred 1 to 10 carbon atoms, yet even more preferred 1 to 6 carbon atoms and particularly preferred 1 to 3 carbon atoms, such as for instance methyl or ethyl, preferably methyl.

In one of the preferred embodiments, the substituent X¹ in Formula I is —NR¹— and R¹ is hydrogen, the compound of the present invention being represented by Formula II:

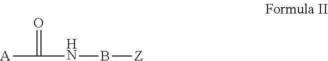

In other preferred embodiments, X¹ in Formula I is —O—. In these embodiments, the compound of the present invention is represented by Formula III shown below:

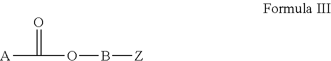

In yet another preferred embodiment, X¹ in Formula I is —S— and the compound of the present invention is thus represented by Formula IV:

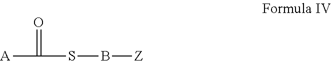

The substituent X² in R¹⁰ of Formula A-II can be —O—, —S— or —NR¹³—, R¹³ being hydrogen or an alkyl substituent having 1 to 3 carbon atoms. Preferably, R¹³ is hydrogen.

In some embodiments, A is represented by

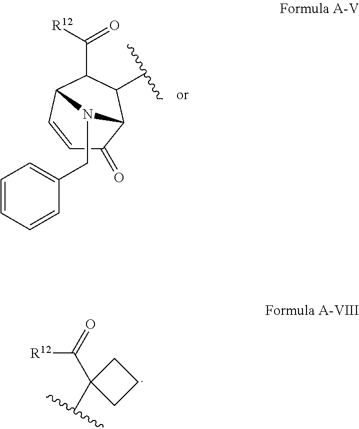

In these embodiments, R¹² is represented by hydroxy, —B—Z or

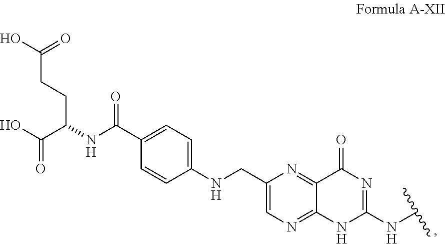 [SMILES: *Nc1nc(=O)c2nc(CNc3ccc(C(=O)N[C@@H](CCC(=O)O)C(=O)O)cc3)cnc2[nH]1]

whereby —B— and —Z are as specified for Formula I above.

The substituent represented by Formula A-XII is a folic acid residue. Without wishing to be bound by any theory it is believed that compounds of the present invention having R¹² represented by Formula A-XII have a particularly strong activity against lung and brain cancer. In particular, the activity against lung and brain cancer of compounds in which R¹² is represented by Formula A-XII is usually higher than activity against lung cancer of corresponding compounds in which R¹² is hydroxyl substituent.

In one embodiment A is represented by Formula A-I or A-IV, X¹ is —O— and —B—Z is not —(CH₂)₄—O—P(O)(OC₂H₅)₂.

In another embodiment A is represented by Formula A-II and X¹ is not —O— and/or —B— is an aliphatic substituent with 1 to 100, preferably with 1 to 42 carbon atoms.

The substituent B in Formula I is

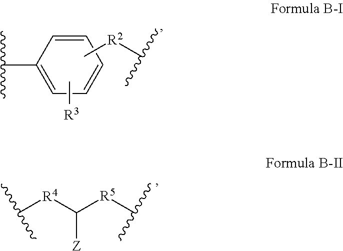

a single bond or an aliphatic substituent, preferably with 1 to 100, more preferred with 1 to 42 and particularly preferred with 1 to 22 carbon atoms, preferably with 1 to 15 carbon atoms, more preferred with 1 to 10 carbon atoms and particularly preferred with 1 to 6 carbon atoms.

Substituents R², R⁴, and R⁵ can be the same or different alkylene substituent having 1 to 3 carbon atoms. Preferably the substituent R² in Formula B-I is selected from the group consisting of methylene, ethylene and trimethylene; in a more preferred embodiment, R² is methylene or ethylene, whereby methylene is particularly preferred.

In yet another preferred embodiment, the substituent B is represented by Formula B-II and R⁴, and R⁵ are identical alkylene substituent having 1 to 3 carbon atoms. In a particularly preferred embodiment, R⁴ and R⁵ are both methylene substituents so that the substituent B in Formula I is represented by Formula B-IV:

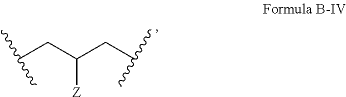

Z being defined below.

In a particularly preferred embodiment, the substituent B forms a glycerol ester residue together with X¹ and Z.

R³ in Formula B-I can be hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)O—C₁₋₆-alkyl, —OC(O)—C₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxy substituents. In some preferred embodiments, R³ in Formula B-I is selected from hydrogen, an alkyl having 1 to 3 carbon atoms, halo and methoxy. Preferably, R³ is selected from the group consisting of hydrogen, methyl, fluoro, chloro, bromo and methoxy, preferably from the group consisting of hydrogen, methyl, chloro and fluoro. In a particularly preferred embodiment, R³ represents hydrogen so that the substituent B is represented by Formula B-III:

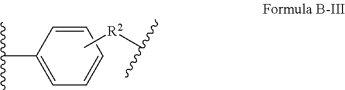

The substitution pattern of the substituent B in Formulae B-I and B-III is not particularly limited. When the substituent B is represented by Formula B-III the aromatic moiety of the substituent B can be 1,2- or 1,3- or 1,4-substituted. Preferably, the aromatic moiety of B is 1,4-substituted so that B is represented by Formula B-V:

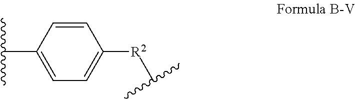

In yet another embodiment of the present invention the substituent B is an aliphatic substituent, preferably having 1 to 22 carbon atoms, more preferred 1 to 6 carbon atoms. In a particularly preferred embodiment, B is selected from the group consisting of alkylene substituents with 1 to 6 carbon atoms, alkenylene substituent having 2 to 6 carbon atoms and alkynylene substituent having 2 to 6 carbon atoms. In a more preferred embodiment, the substituent B is an alkylene substituent with 1 to 6 carbon atoms, preferably an alkylene substituent with 1 to 4 carbon atoms, even more preferred tetramethylene substituent.

The substituent Z in Formula I is selected from the group consisting of

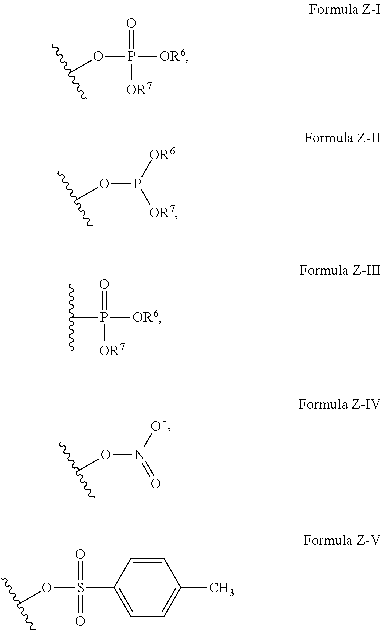

and a folic acid residue;
R⁶ being independently selected from hydrogen, C₁₋₁₀₀-alkyl, preferably C₁₋₆-alkyl, and polyethylene glycol substituent, R⁷ being independently selected from hydrogen, C₁₋₁₀₀-alkyl, preferably C₁₋₆-alkyl, and polyethylene glycol substituent.

The folic acid residue is preferably selected from the one of the following:

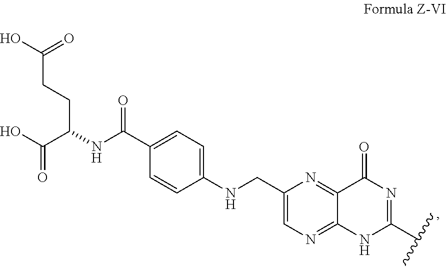 [SMILES: *c1nc(=O)c2nc(CNc3ccc(C(=O)N[C@@H](CCC(=O)O)C(=O)O)cc3)cnc2[nH]1]

In a particularly preferred embodiment of the present invention, X′ is —N— and Z is represented by Formula Z-VI. Accordingly, the compound of the present invention is represented by the Formula V

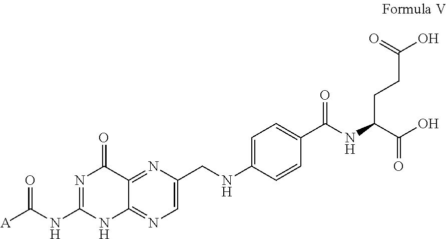

Preferably, the substituent Z is represented by Formula Z-I. In preferred embodiments, R⁶ and R⁷ are identical. Preferably, R⁶ and R⁷ are alkyl substituents having 1 to 42 carbon atoms, more preferred 1 to 22 carbon atoms, even more preferred 1 to 6 carbon atoms, yet even more preferred 1 to 3 carbon atoms, whereby it is most preferred that R⁶ and R⁷ are ethyl substituents.

In yet another preferred embodiment, R⁶ is represented by hydrogen and R⁷ is polyethylene glycol residue, for instance (OCH₂CH₂)ₙOCH₃, whereby n is from 40 to 50.

In a further embodiment of the present invention, the substituent B together with the substituent Z forms a structure

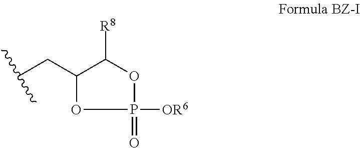

R⁶ being defined as above,
R⁸ being independently selected from hydrogen, an aliphatic substituent, preferably with 1 to 22 carbon atoms, more preferred C₁₋₆-alkyl, and a polyethylene glycol residue.

In yet another preferred embodiment, R⁸ is hydrogen and the substituent B together with the substituent Z forms a structure

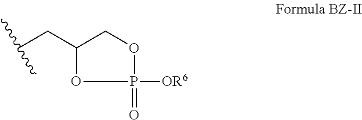

Thus, in some preferred embodiments of the present invention, X¹ is —NR¹—, R¹ is hydrogen; the substituent B is selected from the group consisting of

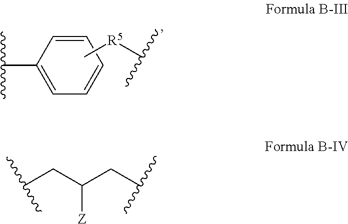

C₁₋₆-alkylene, C₂₋₆-alkenylene and C₂₋ₛ-alkynylene,
the substituent Z is represented by Formula Z-I and
R⁹ being independently selected from hydrogen, C₁₋₃-alkyl and (OCH₂CH₂)ₙOCH₃,
R¹⁰ being independently selected from C₁₋₃-alkyl and (OCH₂CH₂)ₙOCH₃,
whereby n is from 40 to 50.

In another preferred embodiment, X¹ is —NR¹—, R¹ is hydrogen;

the substituent B is selected from the group consisting of C₁₋₄-alkylene and

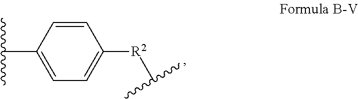

R² being methylene or ethylene; and
Z is represented by Formula Z-I, R⁶ and R⁷ being identical C₁₋₃-alkyl substituents.

In another preferred embodiment, X¹ is —O—;

the substituent B is selected from the group consisting of C₁₋₄-alkylene and

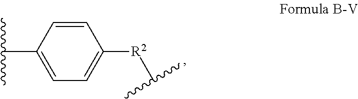

R² being methylene or ethylene; and
the substituent Z is represented by Formula Z-I, R⁶ and R⁷ being identical C₁₋₃-alkyl substituents.

In yet another preferred embodiment, X¹ is —NR¹—, R¹ is hydrogen; the substituent B is —(CH₂)₄—; and the substituent Z is represented by Formula Z-I, R⁶ and R⁷ being identical C₁₋₃-alkyl substituents.

In yet another preferred embodiment, X¹ is —NH—, —S— or —O—; B is —(CH₂)₄—; and the substituent Z is represented by Formula Z-I, R⁶ and R⁷ being identical C₁₋₃-alkyl substituents.

If the substituent A is represented by Formula A-IV, then R² is preferably S(O)CH₃.

In one embodiment of the invention, the substituent A is represented by Formula A-I. The corresponding compounds are structurally related to ibuprofen. Accordingly, the compounds of Formula I include but are not limited to compounds 1 to 8, the structures of which are shown below:

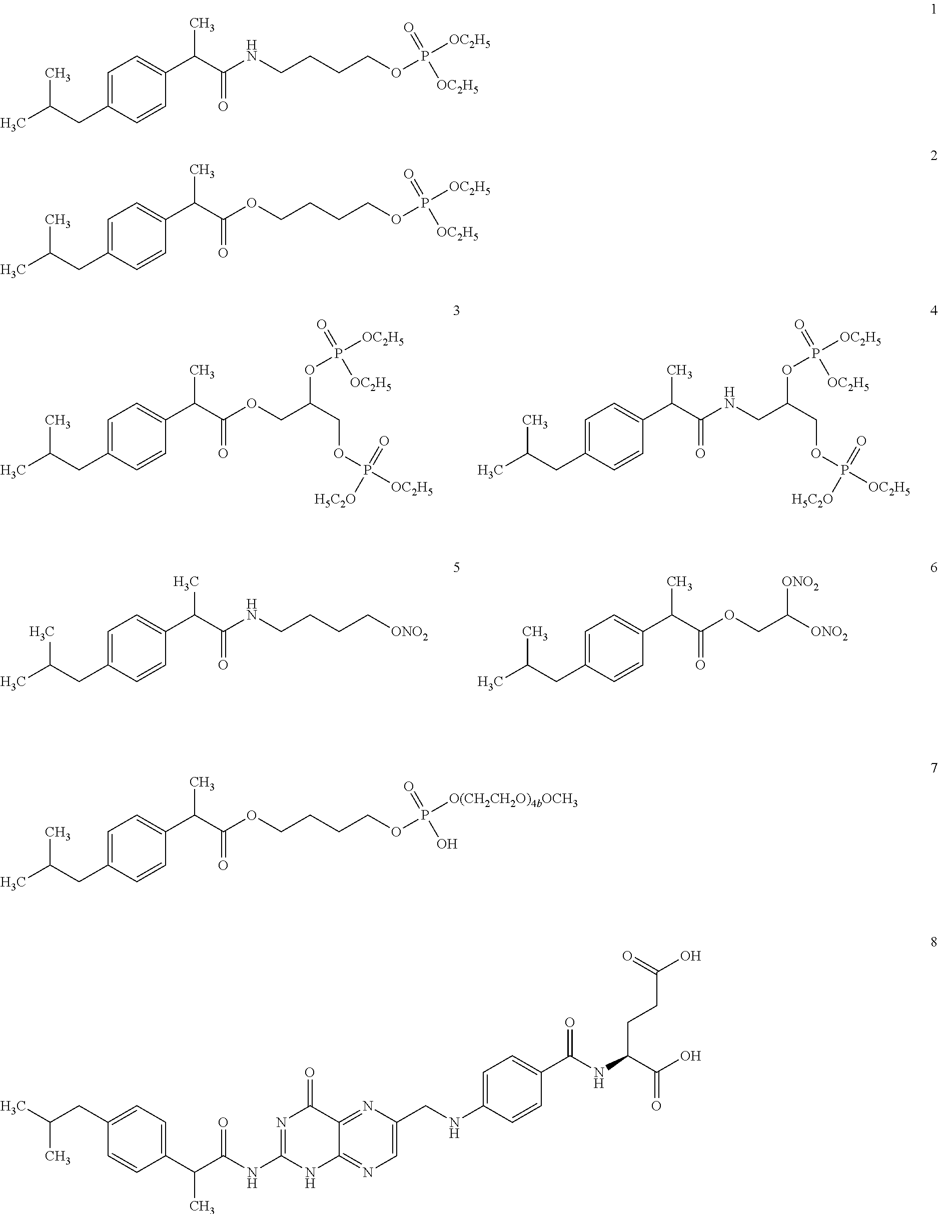

In another embodiment of the invention, the substituent A is represented by Formula A-II, R⁹ is hydrogen and R¹⁰ is —X²—C(O)—CH₃. Thus, the corresponding compounds are structurally related to Aspirin®. The corresponding compounds of Formula I include but are not limited to the following compounds 9 to 32:

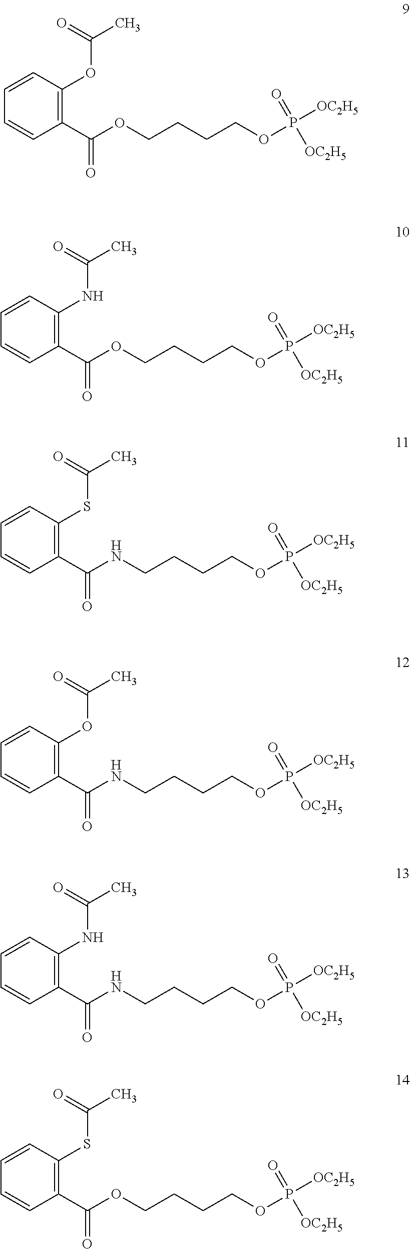 [SMILES: CCOP(=O)(OCC)OCCCCOC(=O)c1ccccc1OC(C)=O ; CCOP(=O)(OCC)OCCCCOC(=O)c1ccccc1NC(C)=O ; CCOP(=O)(OCC)OCCCCNC(=O)c1ccccc1SC(C)=O ; CCOP(=O)(OCC)OCCCCNC(=O)c1ccccc1OC(C)=O ; CCOP(=O)(OCC)OCCCCNC(=O)c1ccccc1NC(C)=O ; CCOP(=O)(OCC)OCCCCOC(=O)c1ccccc1SC(C)=O]

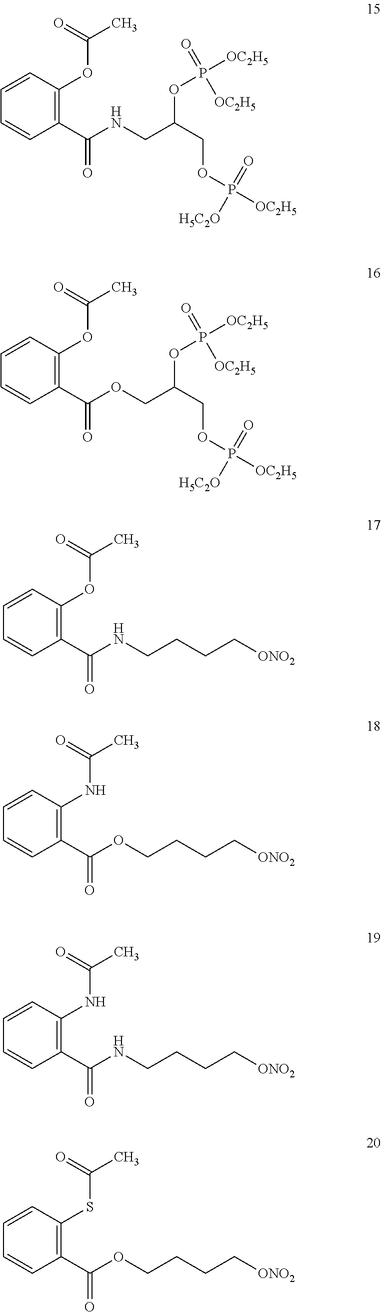

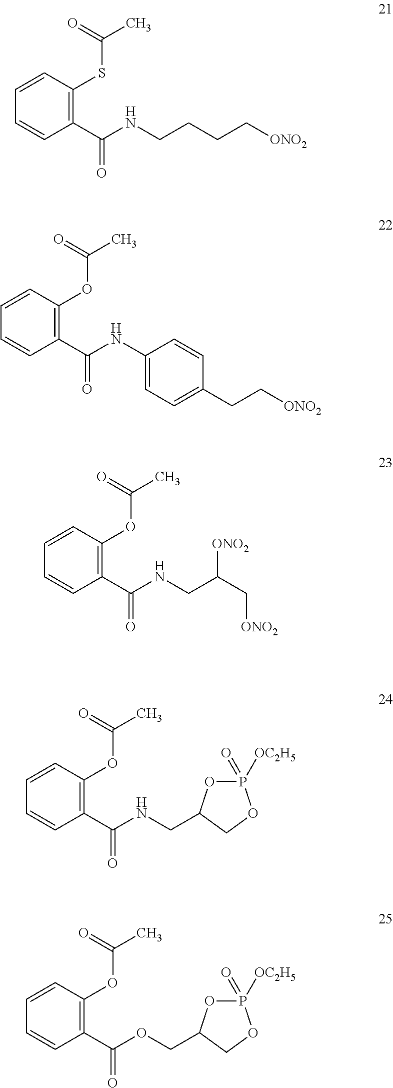

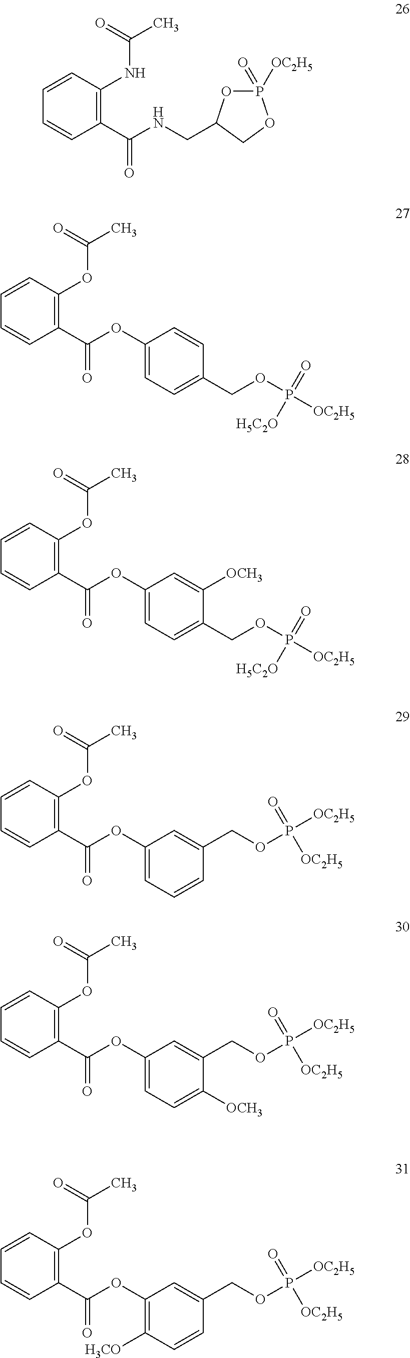

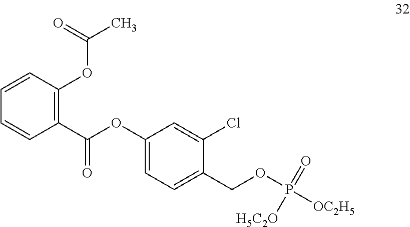

In a further embodiment of the invention, the substituent A is represented by Formula A-II, R⁹ is hydrogen and R¹⁰ is represented by Formula A-XII, Formula A-XIII or Formula A-XIV. The corresponding compounds of Formula I include but are not limited to the following compounds 33 to 50:

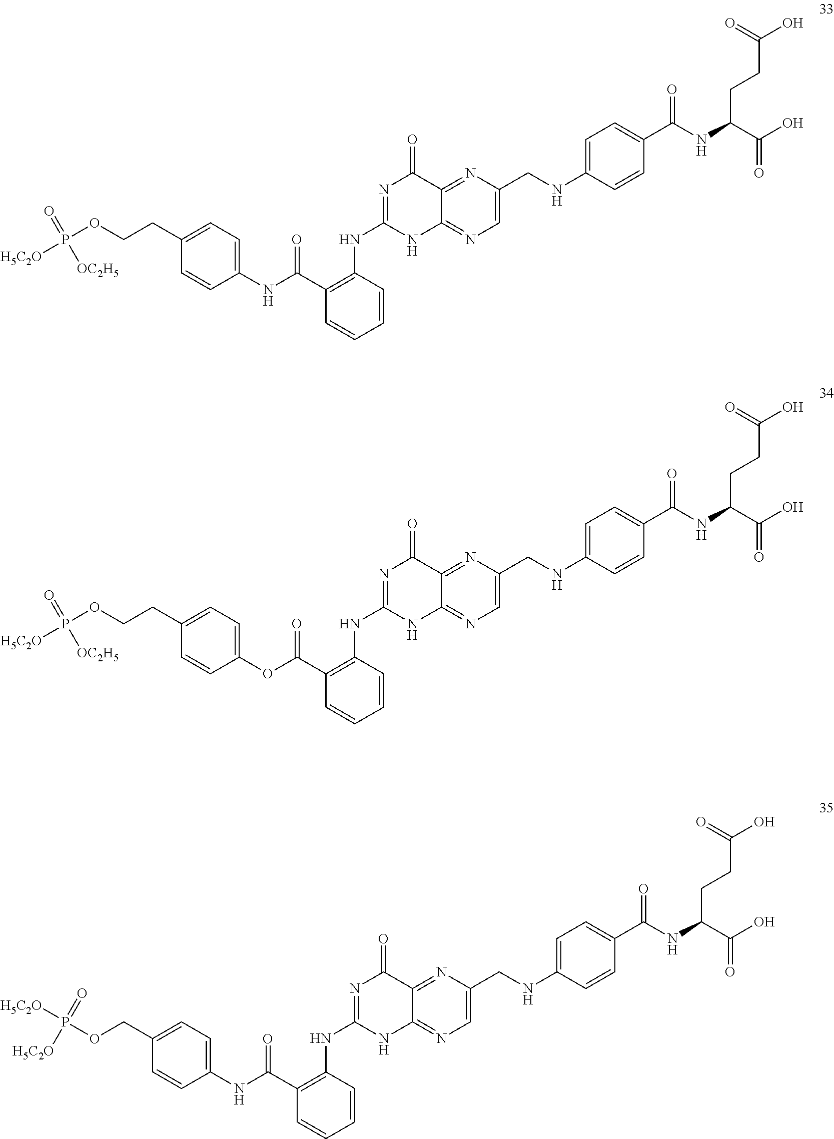

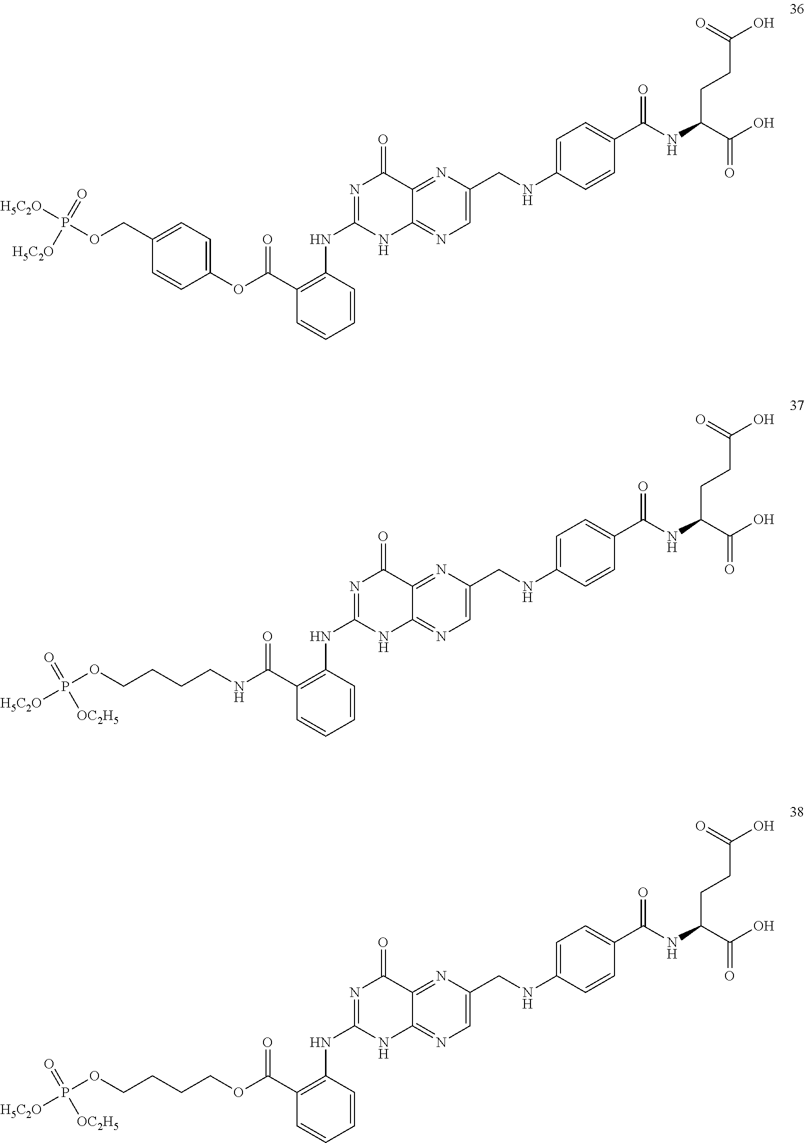

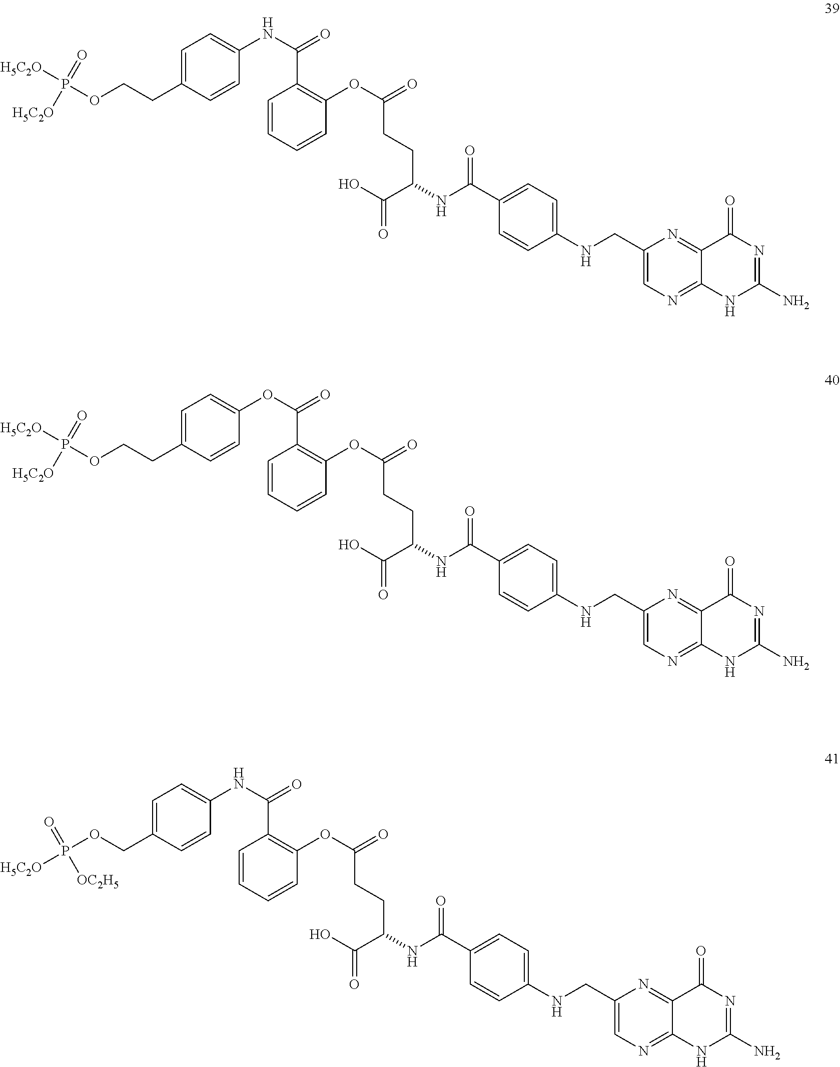

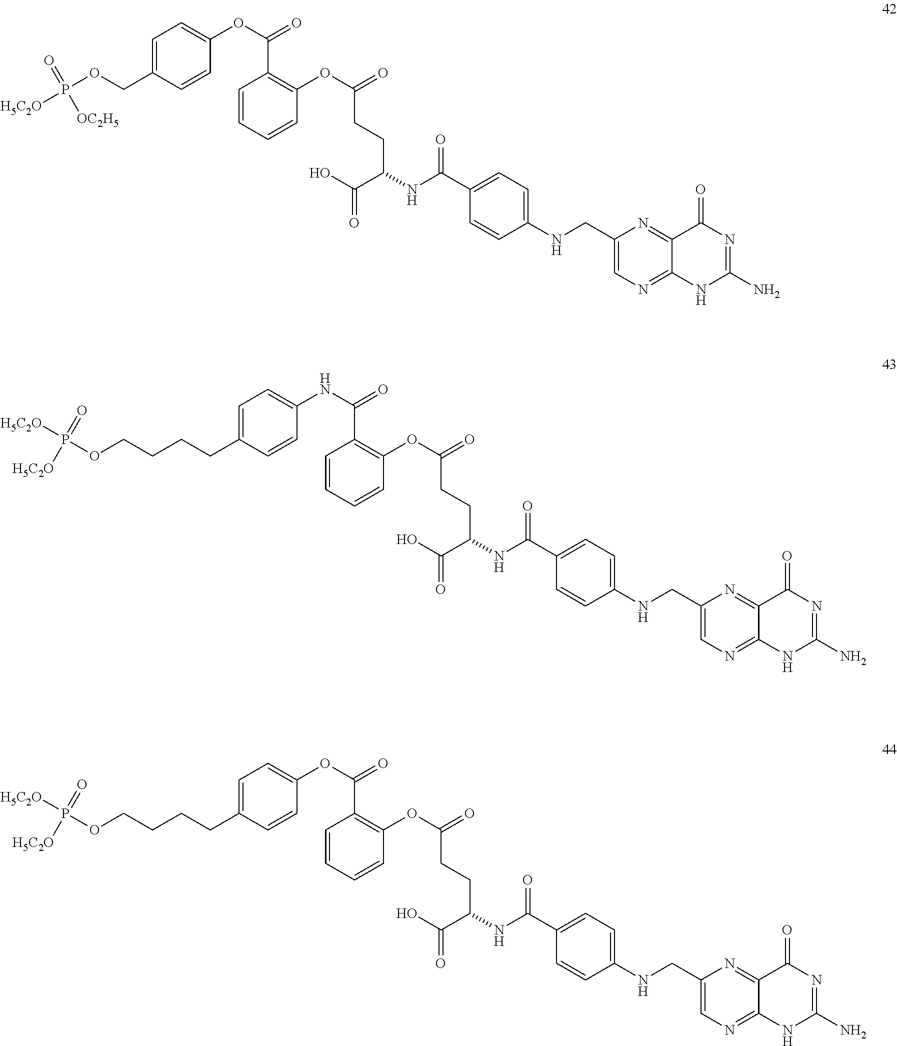

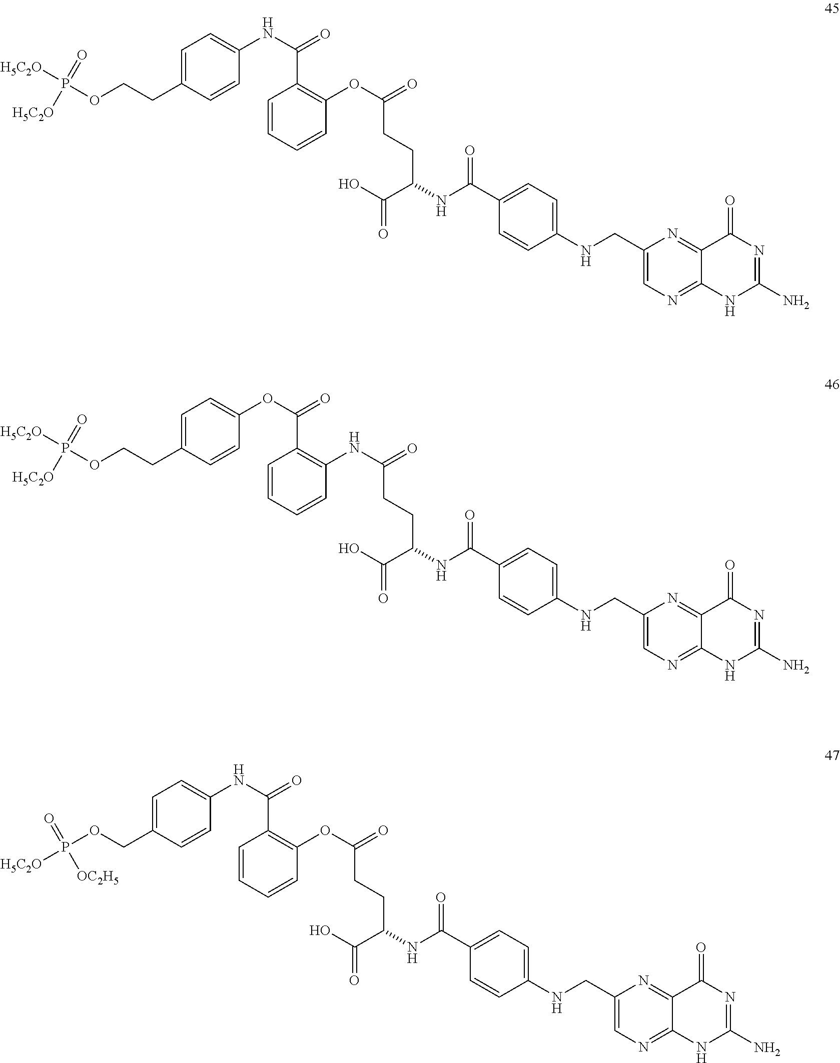

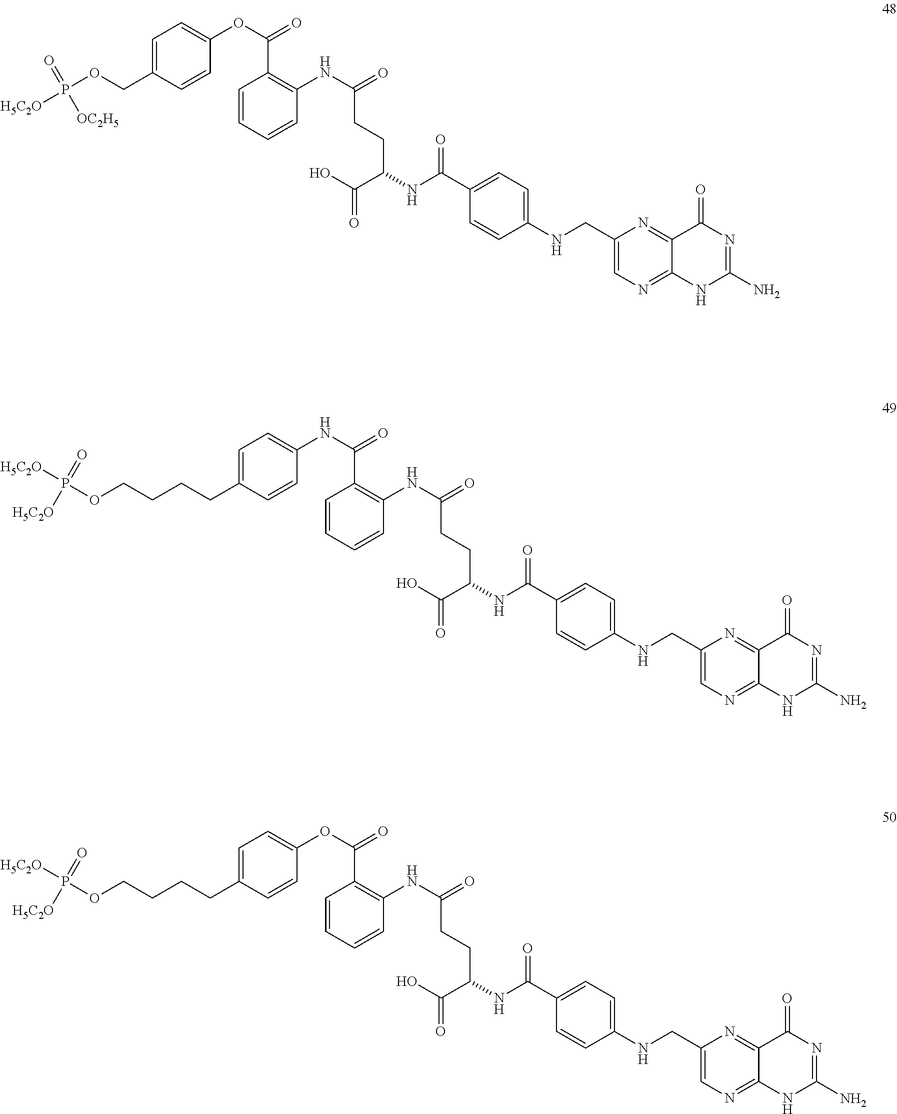

In a further embodiment of the invention, the substituent A is represented by Formula A-II, R⁹ is hydrogen and R¹⁰ is represented by Formula A-XV. The corresponding compounds of Formula I include but are not limited to the following compounds 51 to 68:

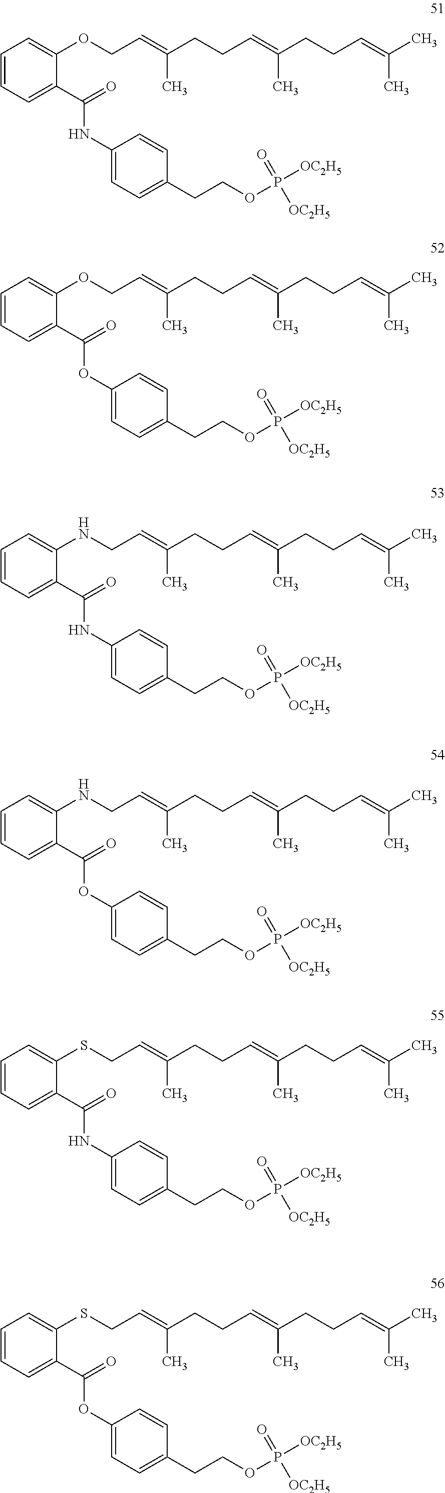 [SMILES: CCOP(=O)(OCC)OCCc1ccc(NC(=O)c2ccccc2OC/C=C(\C)CC/C=C(\C)CCC=C(C)C)cc1 ; CCOP(=O)(OCC)OCCc1ccc(OC(=O)c2ccccc2OC/C=C(\C)CC/C=C(\C)CCC=C(C)C)cc1 ; CCOP(=O)(OCC)OCCc1ccc(NC(=O)c2ccccc2NC/C=C(\C)CC/C=C(\C)CCC=C(C)C)cc1 ; CCOP(=O)(OCC)OCCc1ccc(OC(=O)c2ccccc2NC/C=C(\C)CC/C=C(\C)CCC=C(C)C)cc1 ; CCOP(=O)(OCC)OCCc1ccc(NC(=O)c2ccccc2SC/C=C(\C)CC/C=C(\C)CCC=C(C)C)cc1 ; CCOP(=O)(OCC)OCCc1ccc(OC(=O)c2ccccc2SC/C=C(\C)CC/C=C(\C)CCC=C(C)C)cc1]

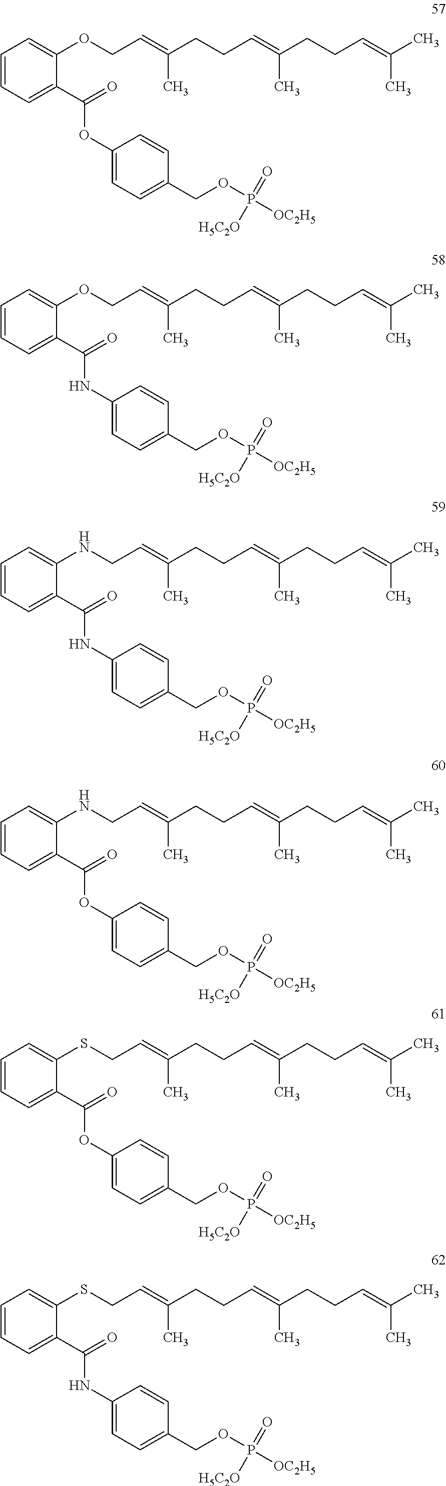

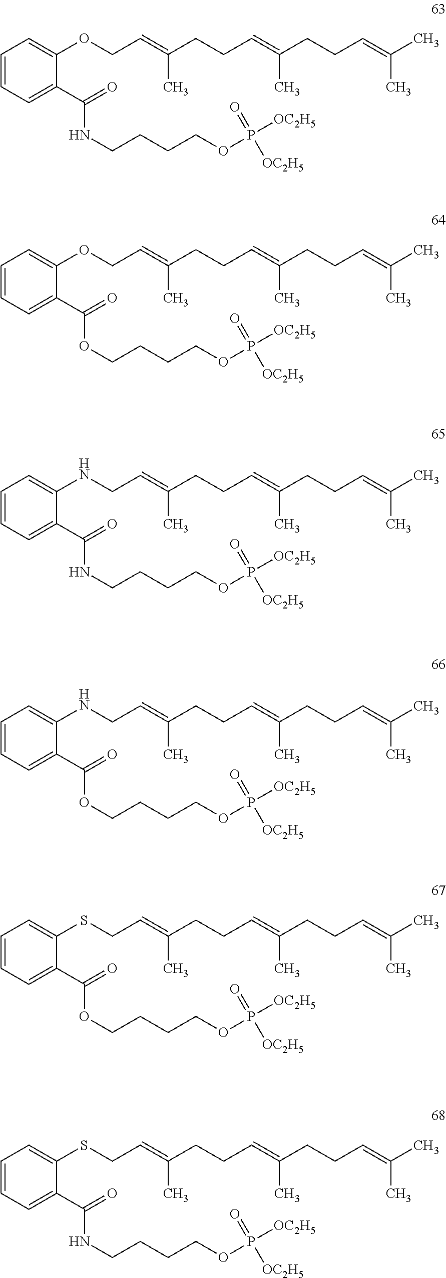 [SMILES: CCOP(=O)(OCC)OCCCCNC(=O)c1ccccc1OC/C=C(\C)CC/C=C(\C)CCC=C(C)C ; CCOP(=O)(OCC)OCCCCOC(=O)c1ccccc1OC/C=C(\C)CC/C=C(\C)CCC=C(C)C ; CCOP(=O)(OCC)OCCCCNC(=O)c1ccccc1NC/C=C(\C)CC/C=C(\C)CCC=C(C)C ; CCOP(=O)(OCC)OCCCCOC(=O)c1ccccc1NC/C=C(\C)CC/C=C(\C)CCC=C(C)C ; CCOP(=O)(OCC)OCCCCOC(=O)c1ccccc1SC/C=C(\C)CC/C=C(\C)CCC=C(C)C ; CCOP(=O)(OCC)OCCCCNC(=O)c1ccccc1SC/C=C(\C)CC/C=C(\C)CCC=C(C)C]

In another embodiment of the invention, the substituent A is represented by Formula A-II, R⁹ is trifluoromethyl and R¹⁰ is —X²—C(O)—CH₃. The corresponding compounds are structurally related to triflusal. According to this embodiment, the compounds of Formula I include but are not limited to the compounds 69 to 74 listed below:

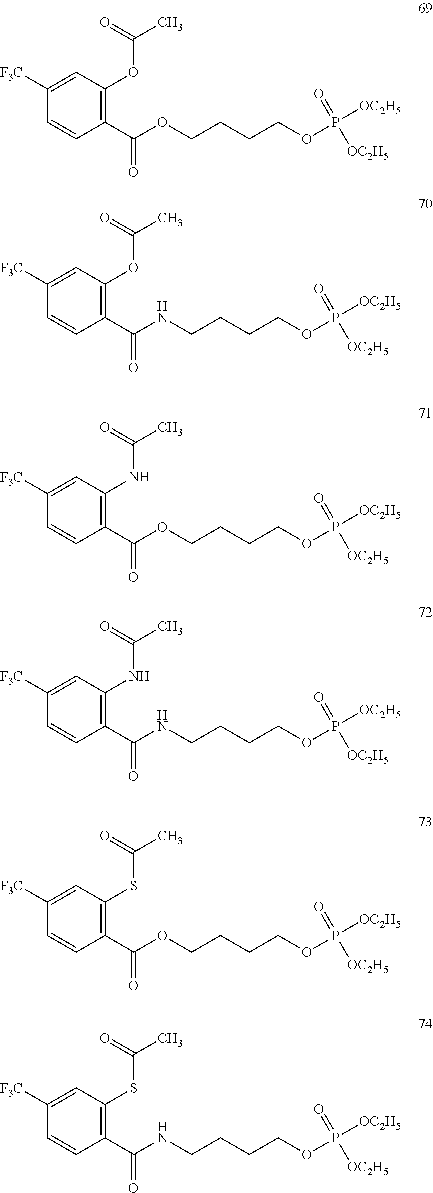 [SMILES: CCOP(=O)(OCC)OCCCCOC(=O)c1ccc(C(F)(F)F)cc1OC(C)=O ; CCOP(=O)(OCC)OCCCCNC(=O)c1ccc(C(F)(F)F)cc1OC(C)=O ; CCOP(=O)(OCC)OCCCCOC(=O)c1ccc(C(F)(F)F)cc1NC(C)=O ; CCOP(=O)(OCC)OCCCCNC(=O)c1ccc(C(F)(F)F)cc1NC(C)=O ; CCOP(=O)(OCC)OCCCCOC(=O)c1ccc(C(F)(F)F)cc1SC(C)=O ; CCOP(=O)(OCC)OCCCCNC(=O)c1ccc(C(F)(F)F)cc1SC(C)=O]

In a further embodiment of the invention, the substituent A is represented by Formula A-II, R⁹ is trifluoromethyl and R¹⁰ is represented by Formula A-XII, Formula A-XIII or Formula A-XIV. The corresponding compounds of Formula I include but are not limited to the following compounds 75 to 92:

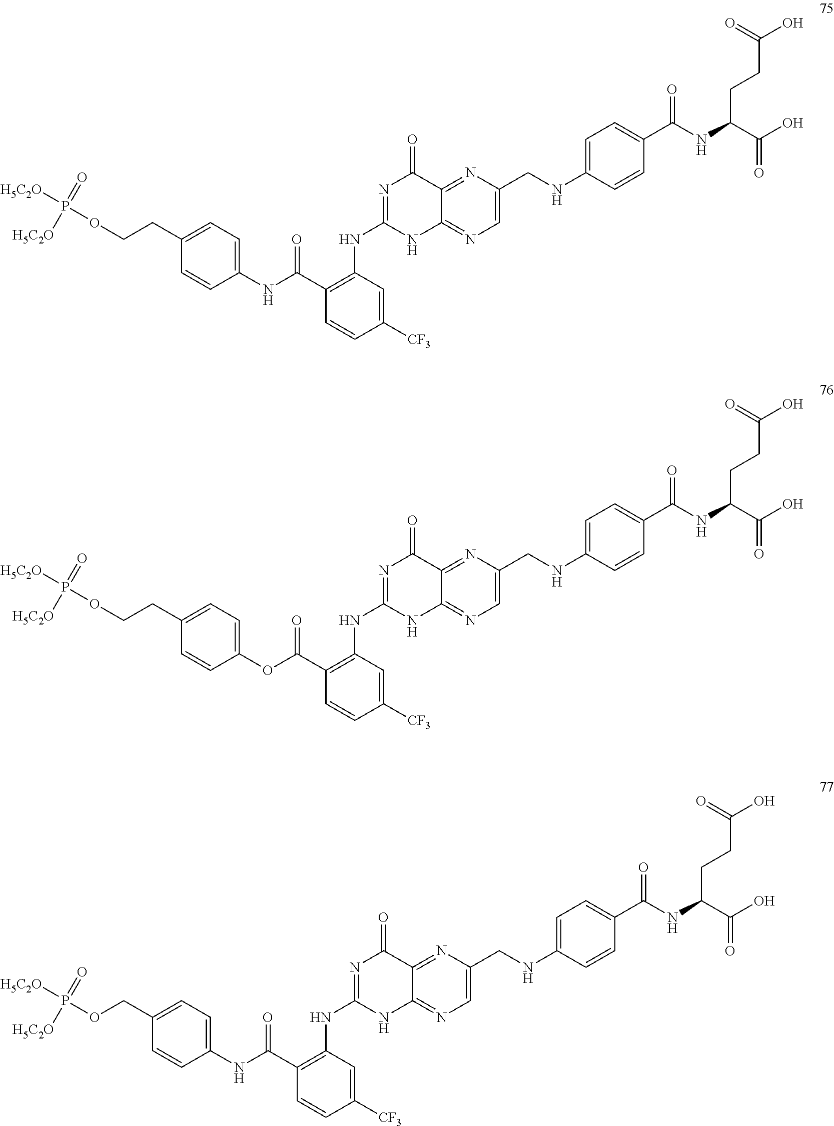

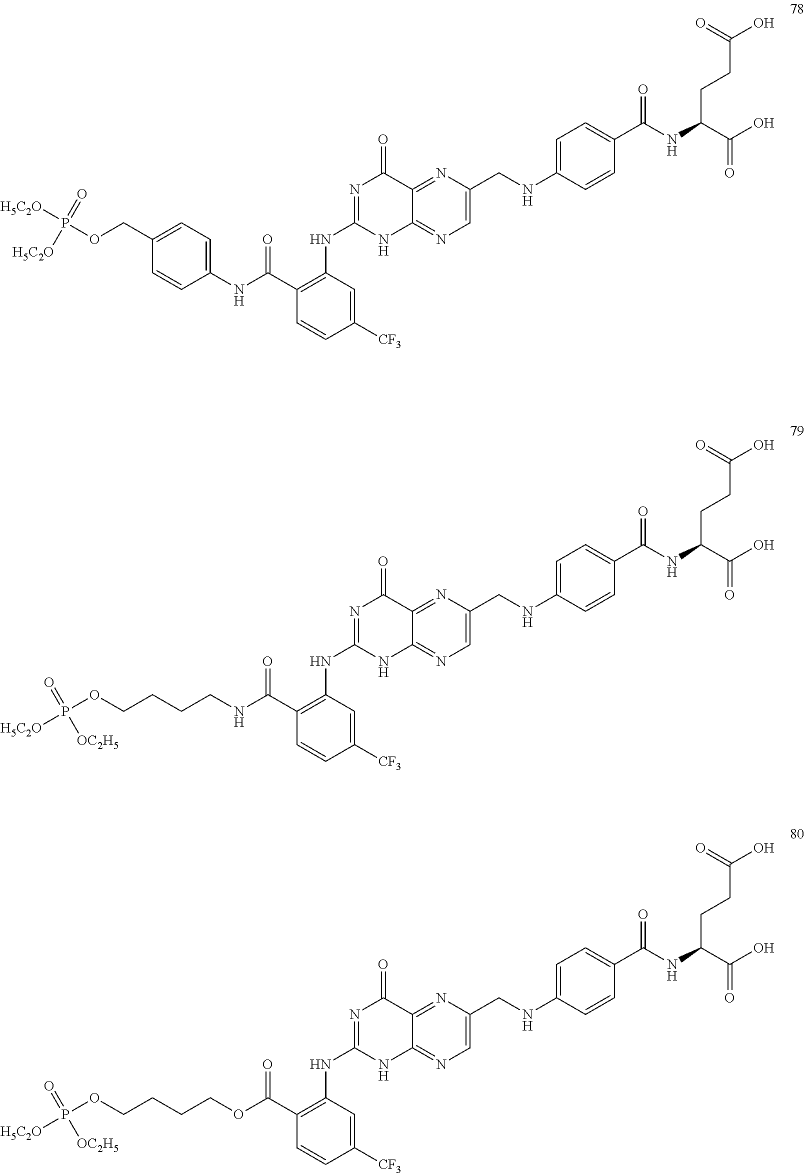

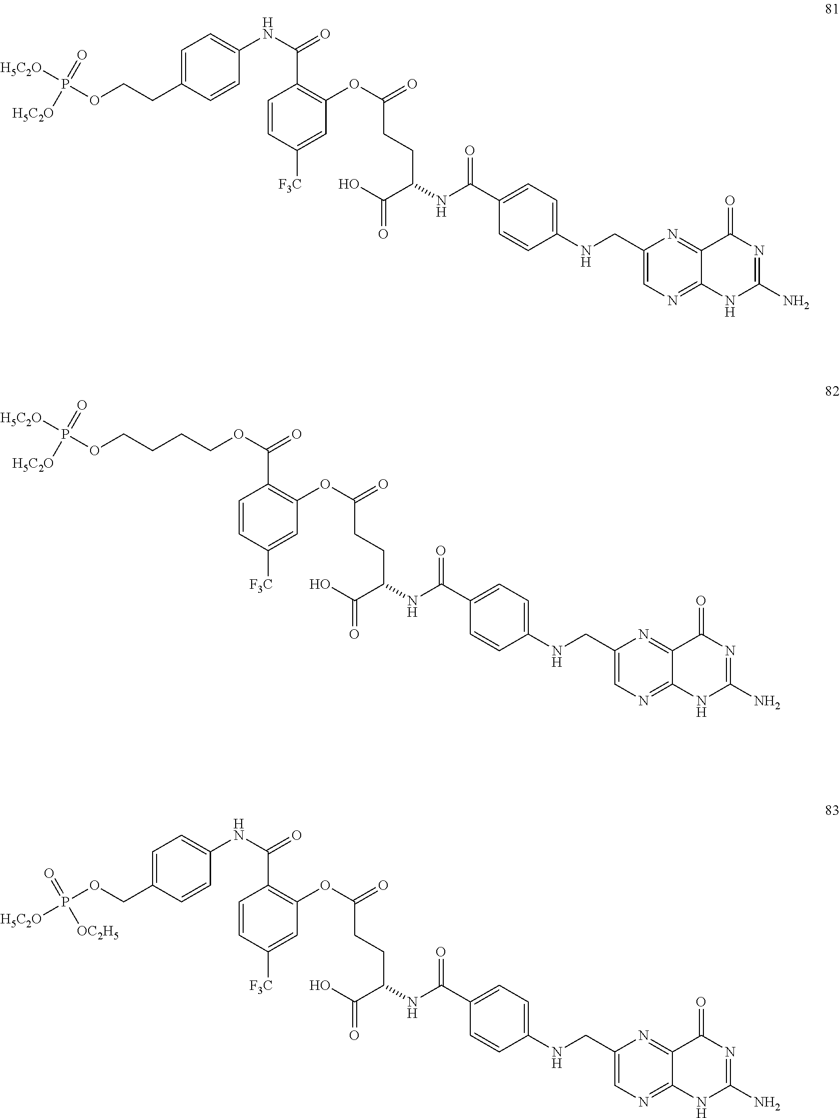

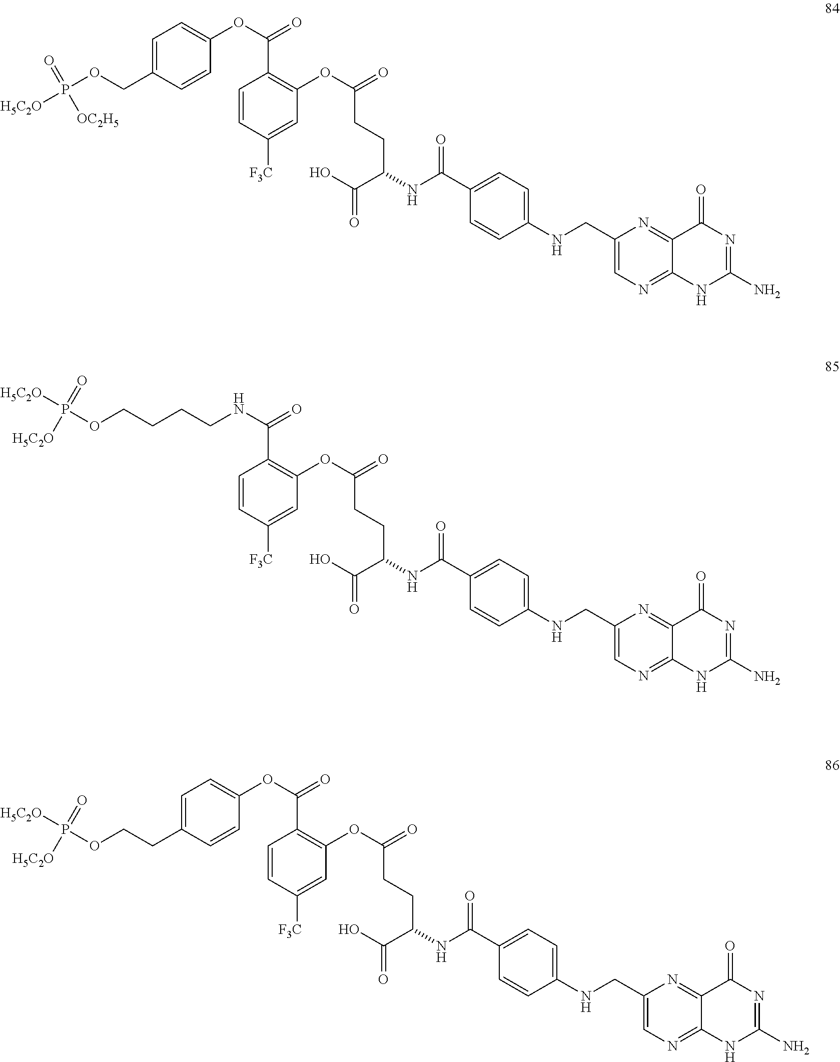

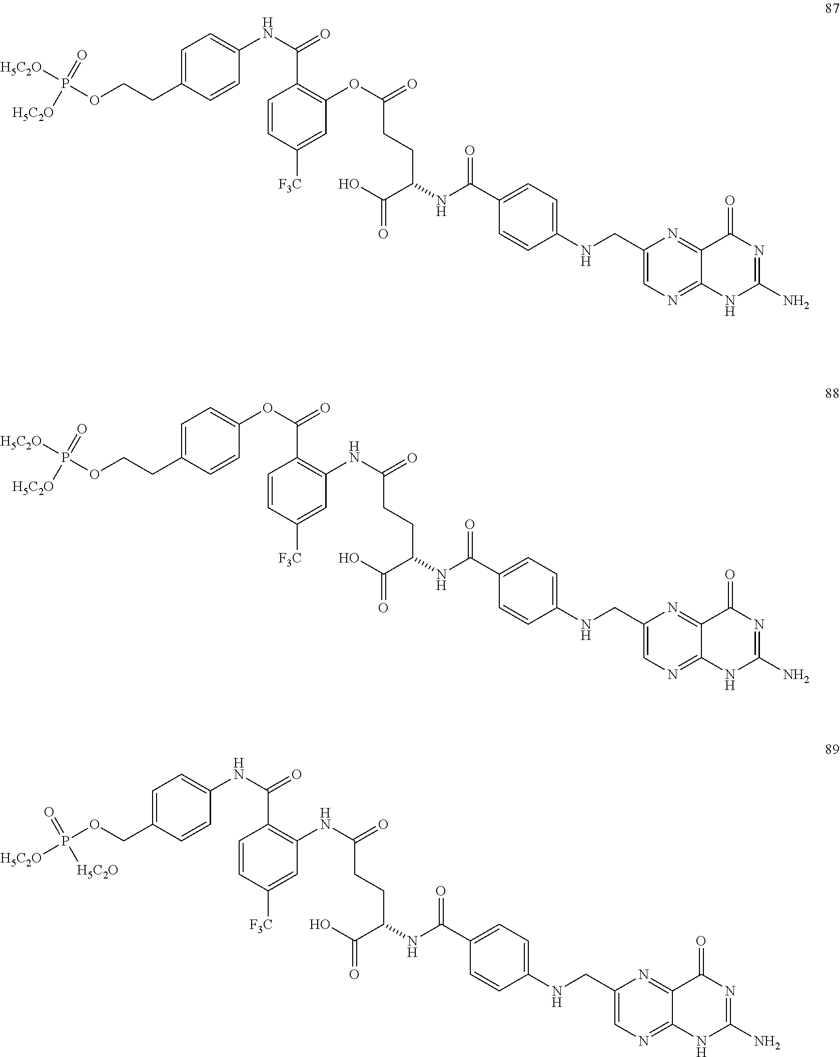

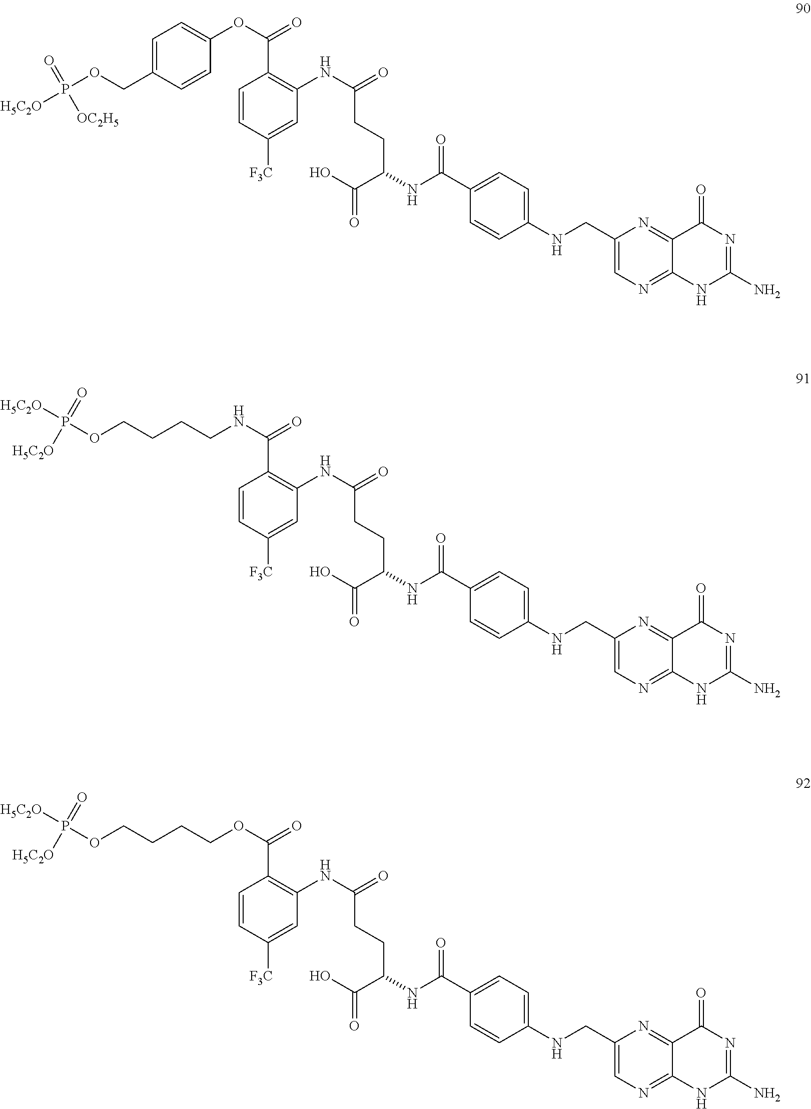

In a further embodiment of the invention, the substituent A is represented by Formula A-IN. The corresponding compounds are structurally related to indomethacin. In this embodiment, the compounds of Formula I include but are not limited to the compounds 93 and 94 shown below:

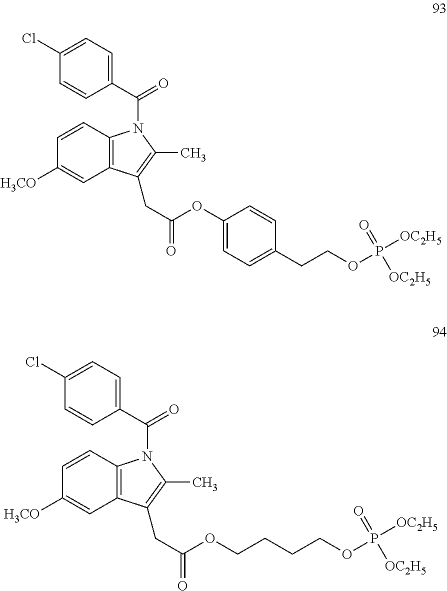 [SMILES: CCOP(=O)(OCC)OCCc1ccc(OC(=O)Cc2c(C)n(C(=O)c3ccc(Cl)cc3)c3ccc(OC)cc23)cc1 ; CCOP(=O)(OCC)OCCCCOC(=O)Cc1c(C)n(C(=O)c2ccc(Cl)cc2)c2ccc(OC)cc12]

Yet another embodiment of the present invention provides compounds of Formula I in which the substituent A is represented by Formula A-IV. Preferably, R¹¹ is S(O)CH₃. The corresponding compounds are structurally related to sulindac. These compounds include but are not limited to the compounds 95 to 100 listed below:

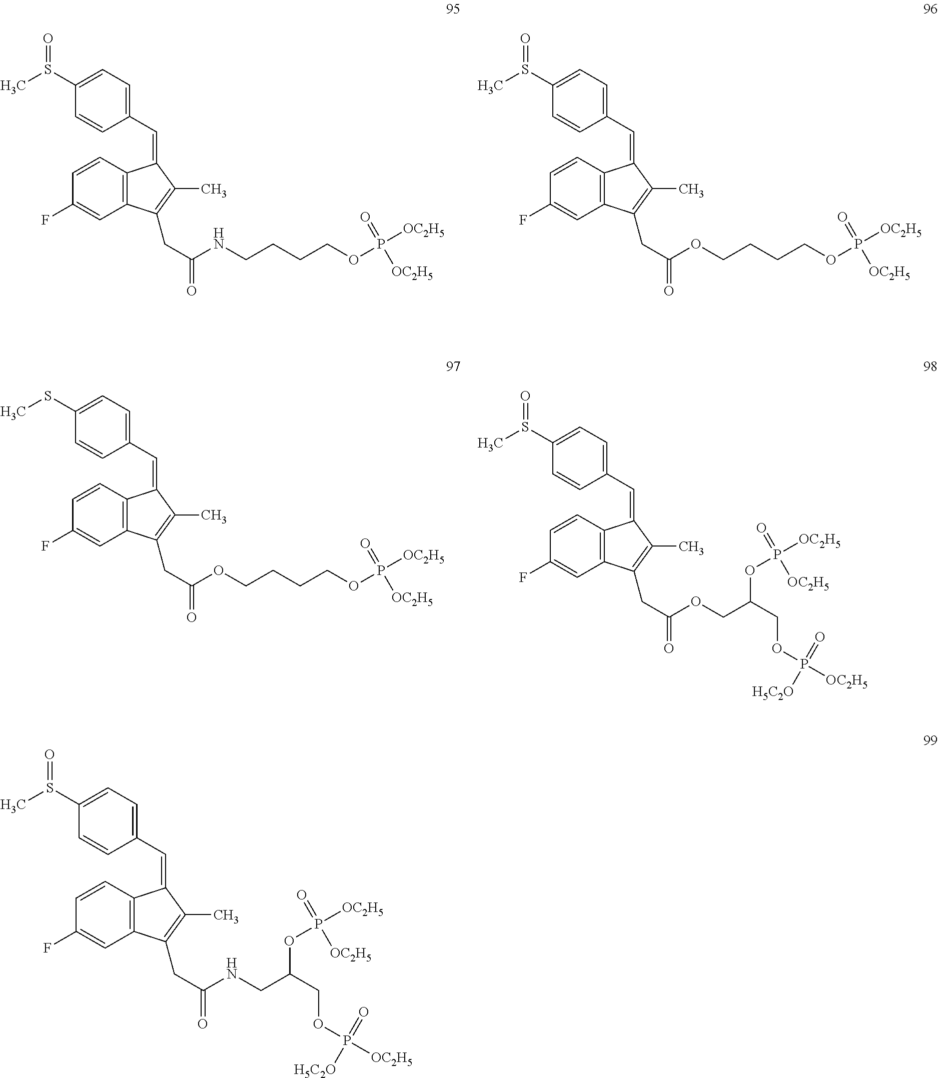

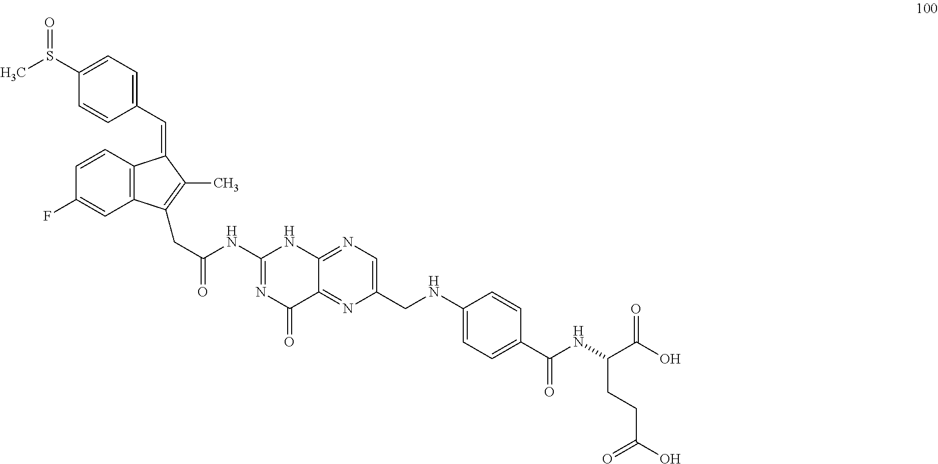

Compounds phospho-sulindac (PS, 96) and phospho-sulindac II 97 have a strong activity against lung and brain cancer and can be administered to human by the respiratory route for the purpose of treatment and/or prevention of lung and brain cancer and precancerous conditions thereof.

In a further embodiment of the invention, the substituent A is represented by Formula A-V. The corresponding compounds of Formula I include but are not limited to the compounds 101 to 112 shown below:

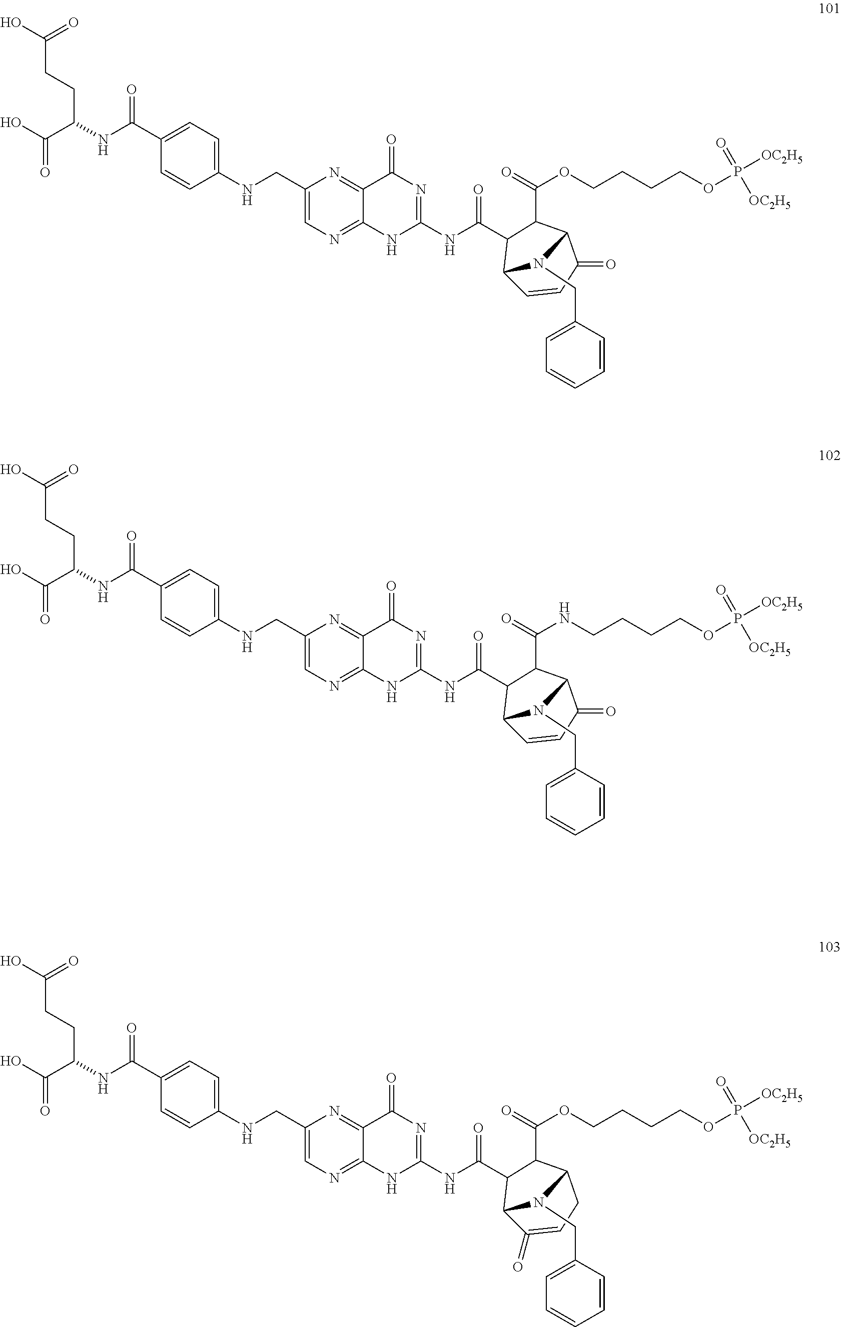

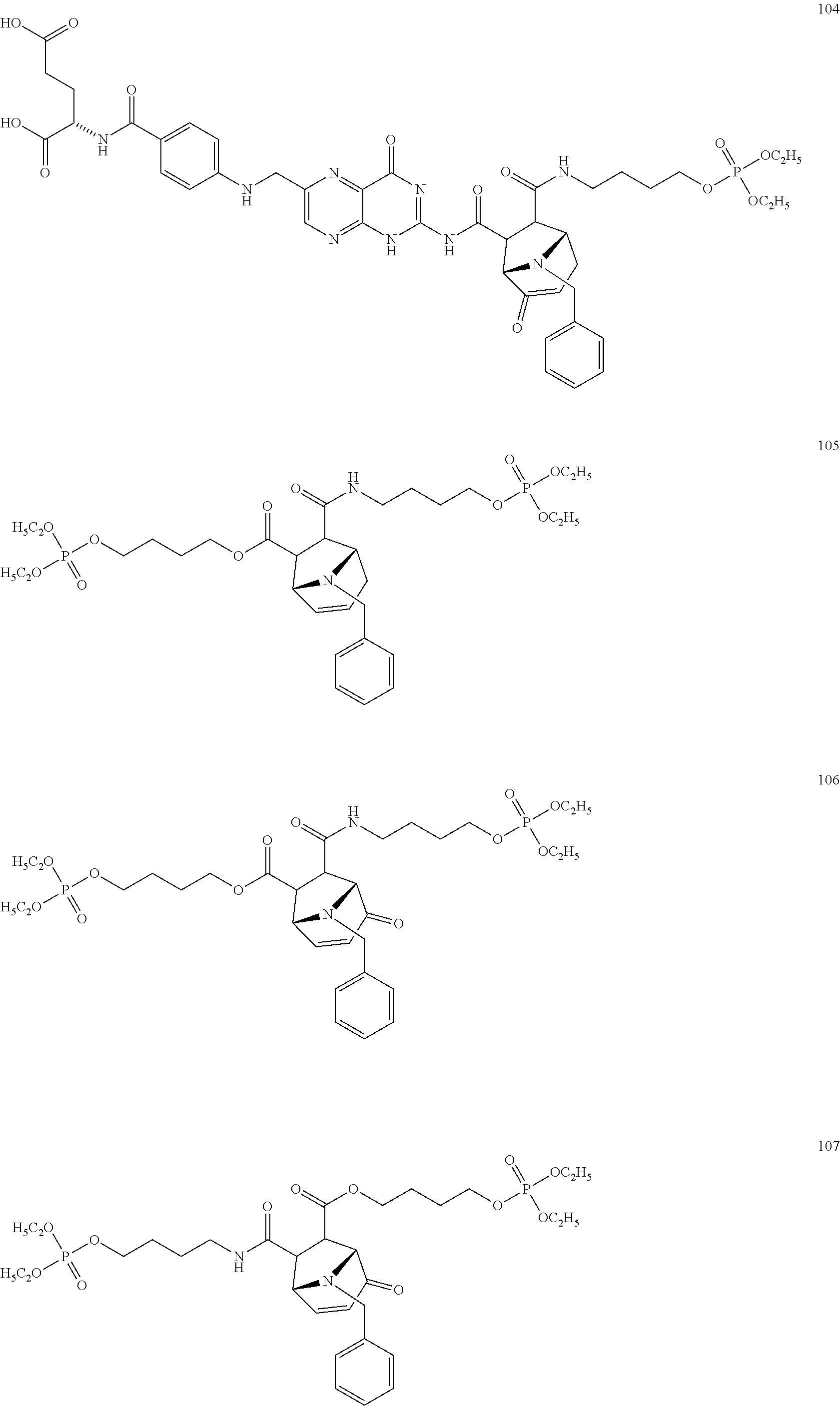

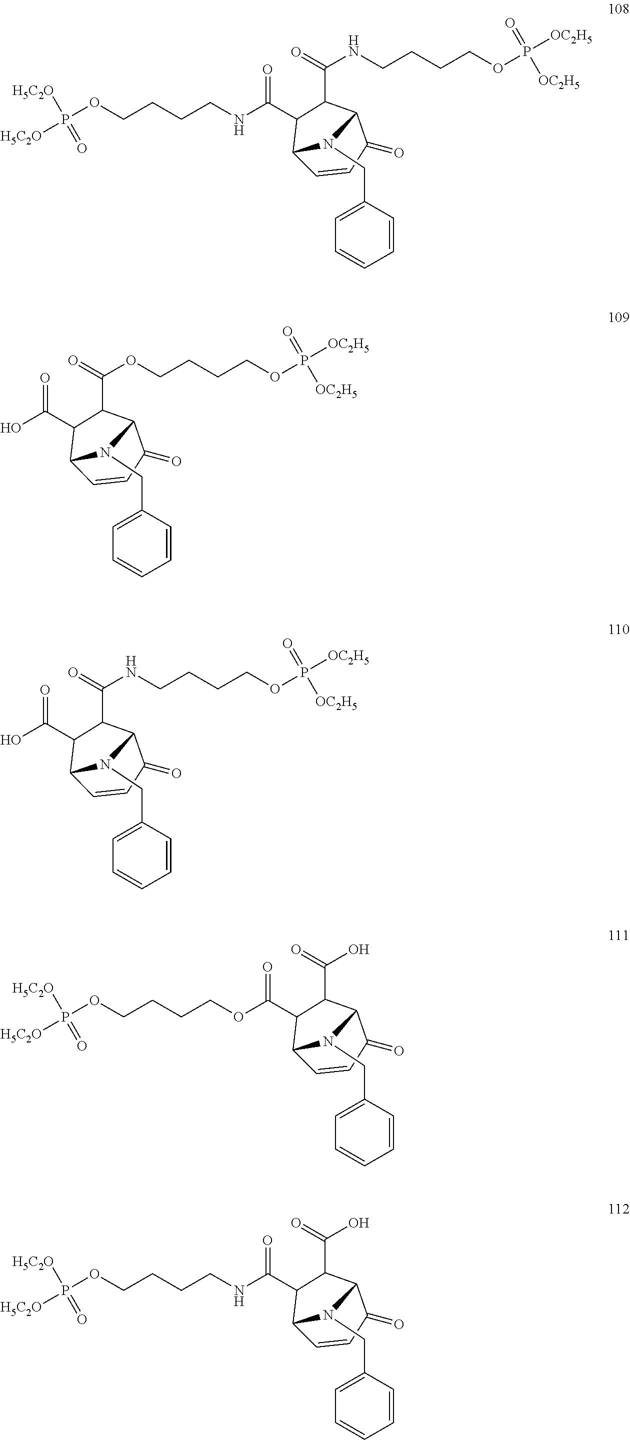

In a further embodiment of the invention, the substituent A is represented by Formula A-VI. These compounds are structurally related to rigosertib. In this embodiment, the compounds of Formula I include but are not limited to the compounds 113 and 114 shown below:

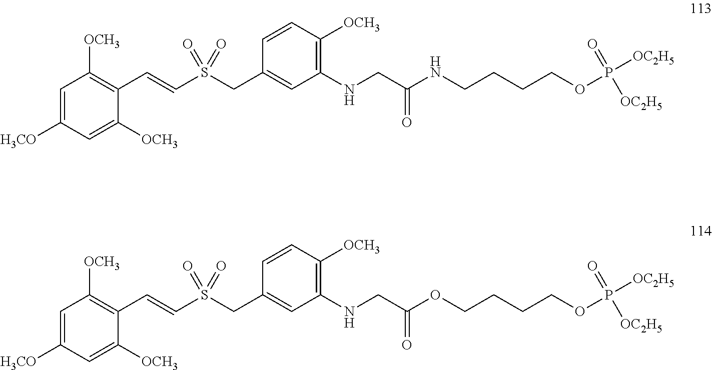 [SMILES: CCOP(=O)(OCC)OCCCCNC(=O)CNc1cc(CS(=O)(=O)/C=C/c2c(OC)cc(OC)cc2OC)ccc1OC ; CCOP(=O)(OCC)OCCCCOC(=O)CNc1cc(CS(=O)(=O)/C=C/c2c(OC)cc(OC)cc2OC)ccc1OC]

In a further embodiment of the invention, the substituent A is represented by Formula A-VII. Thus, the corresponding compounds are structurally related to valproic acid. The corresponding compounds are particularly suitable for the treatment of brain cancer and precancerous conditions of brain cancer, for instance for the treatment of glioma. In this embodiment, the compounds of Formula I include but are not limited to the compounds 115 to 118 shown below:

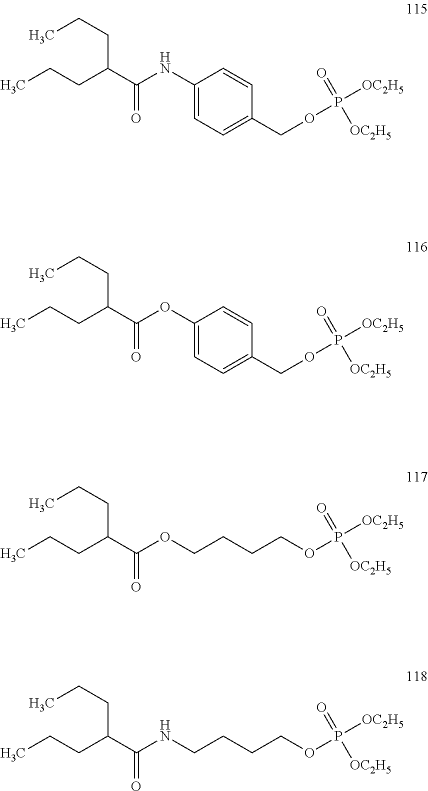 [SMILES: CCCC(CCC)C(=O)Nc1ccc(COP(=O)(OCC)OCC)cc1 ; CCCC(CCC)C(=O)Oc1ccc(COP(=O)(OCC)OCC)cc1 ; CCCC(CCC)C(=O)OCCCCOP(=O)(OCC)OCC ; CCCC(CCC)C(=O)NCCCCOP(=O)(OCC)OCC]

In a further embodiment of the invention, the substituent A is represented by Formula A-VM. In this embodiment, the compounds of Formula I include but are not limited to the compounds 119 and 120 shown below:

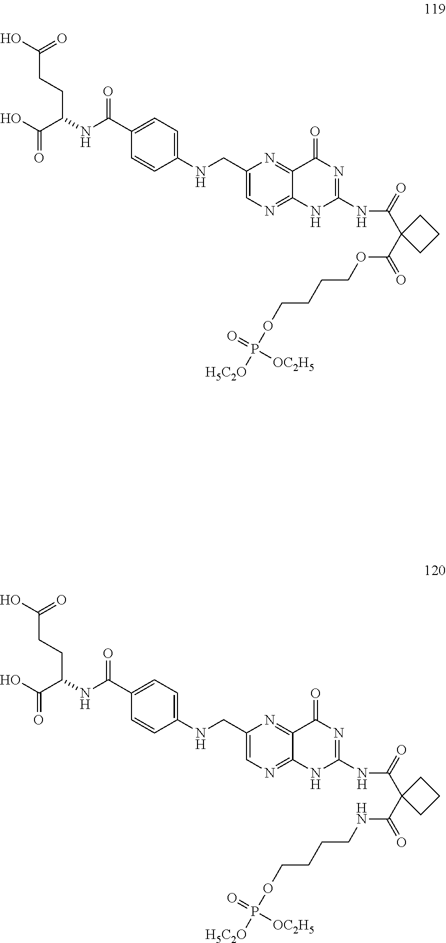

In a further embodiment of the invention, the substituent A is represented by Formula A-IX. Thus, the corresponding compounds are structurally related to naproxen. In this embodiment, the compounds of Formula I include but are not limited to the compounds such as phospho-naproxen 121, the structure of which is shown below:

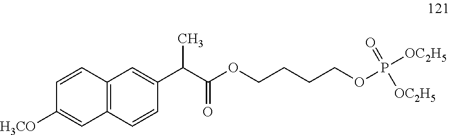 [SMILES: CCOP(=O)(OCC)OCCCCOC(=O)C(C)c1ccc2cc(OC)ccc2c1]

In a further embodiment of the invention, the substituent A is represented by Formula A-X. Thus, the corresponding compounds are structurally related to flurbiprofen. In this embodiment, the compounds of Formula I include but are not limited to the compounds such as phospho-flurbiprofen 122, the structure of which is shown below:

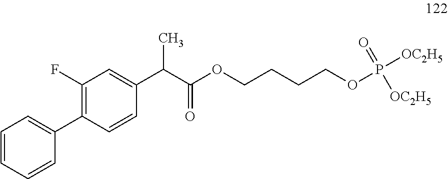 [SMILES: CCOP(=O)(OCC)OCCCCOC(=O)C(C)c1ccc(-c2ccccc2)c(F)c1]

In yet another embodiment of the invention, the substituent A is represented by Formula A-XI and thus the corresponding compounds are structurally related to salinomycin. In this embodiment, the compounds of Formula I include but are not limited to the compounds 123 and 124 shown below:

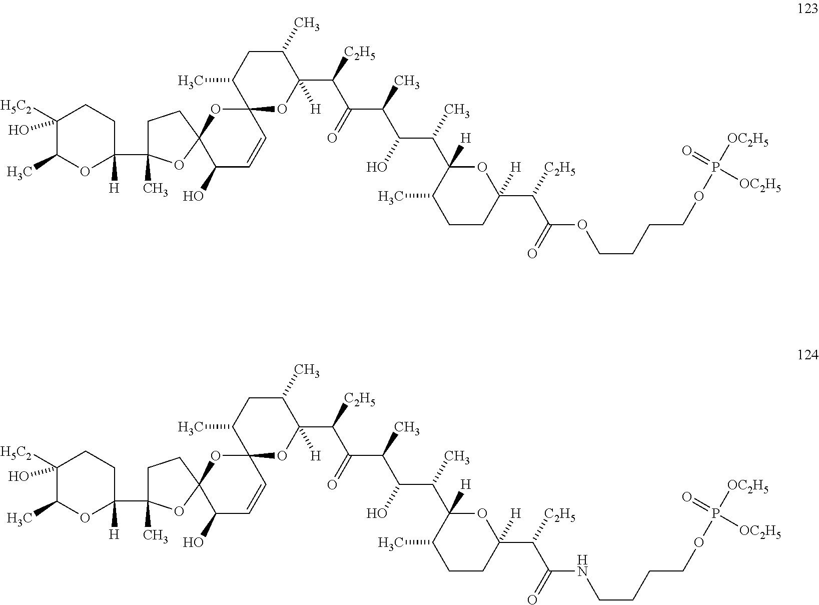

In some preferred embodiments of the present invention, the compound of Formula I is selected from the following: 2-acetoxy-benzoic acid 4-(diethoxy-phosphoryloxymethyl)-phenyl ester (27), 2-acetoxy-benzoic acid 3-(diethoxy-phosphoryloxymethyl)-phenyl ester (29), and phospho-sulindac 96, phospho-sulindac II 97, phospho-flurbiprofen 122, phospho-ibuprofen 2, phosphoaspirin 125, phosphoraspirin II 16, and phosphovalproic acid 116.

In other preferred embodiments of the present invention, the compound of Formula I is selected from the compounds 2, 3, 7, 9, 93, 94, 96, 97 and 98.

In yet other preferred embodiments of the present invention, the compound of Formula I is selected from the compounds 1, 4, 12, 15, 95 and 99.

Some of the compounds of the present invention can comprise one or more stereogenic centers, and thus can exist in various isomeric forms, e.g. stereoisomers and/or diastereomers. Thus, the compounds of Formula I and pharmaceutical compositions thereof may be in the form of an individual enantiomer, diastereomer or geometric isomer, or may be in the form of a mixture of stereoisomers. In certain embodiments, the compounds of the invention are enantiopure compounds. In certain other embodiments, mixtures of stereoisomers or diastereomers are provided. Moreover, when compounds of the invention exist in tautomeric forms, each tautomer is embraced herein.

Furthermore, certain compounds, as described herein may have one or more double bonds that can exist as either the Z or E isomer, unless otherwise indicated. The invention additionally encompasses the compounds as individual isomers substantially free of other isomers and alternatively, as mixtures of various isomers, e.g., racemic mixtures of stereoisomers. In addition to the above-mentioned compounds per se, this invention also encompasses pharmaceutically acceptable derivatives of these compounds and compositions comprising one or more compounds of the invention and one or more pharmaceutically acceptable excipients or additives.

The phrase, “pharmaceutically acceptable derivative”, as used herein, denotes any pharmaceutically acceptable salt, ester, or salt or cocrystal of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others prodrugs. A prodrug is a derivative of a compound, usually with significantly reduced pharmacological activity, which contains at least one additional moiety, which is susceptible to removal in vivo yielding the parent molecule as the pharmacologically active species. An example of a prodrug is an ester, which is cleaved in vivo to yield a compound of interest. Prodrugs of a variety of compounds, and materials and methods for derivatizing the parent compounds to create the prodrugs, are known and may be adapted to the present invention. Certain exemplary pharmaceutical compositions and pharmaceutically acceptable derivatives will be discussed in more detail herein below.

According to the present invention, the compounds of Formula I are active against lung and/or brain cancer and therefore can be used in the treatment and/or prevention of lung and/or brain cancer and precancerous conditions thereof, wherein said compound is administered to a human or animal by the respiratory route. As used herein, “preventing”, “prevention” or “prevent” describes reducing or eliminating the onset of lung or brain cancer or the precancerous conditions thereof or the symptoms or complications of lung and/or brain cancer and precancerous conditions thereof.

Lung cancer can include all forms of cancer of the lung. Lung cancer can include malignant lung neoplasms, carcinoma in situ, typical carcinoid tumors, and atypical carcinoid tumors. Lung cancer can include small cell lung cancer (“SCLC”), non-small cell lung cancer (“NSCLC”), non-squamous non-small cell lung cancer, squamous non-small cell lung cancer, squamous cell carcinoma, non-squamous cell carcinoma, adenocarcinoma, small cell carcinoma, large cell carcinoma, adenosquamous cell carcinoma, and mesothelioma. Lung cancer can include “scar carcinoma,” bronchioalveolar carcinoma, giant cell carcinoma, spindle cell carcinoma, and large cell neuroendocrine carcinoma. Lung cancer can include lung neoplasms having histologic and ultrastructual heterogeneity (e.g. mixed cell types).

The term “brain cancer” as used herein refers to both primary brain tumors and metastatic brain tumors that originate from non-brain cancer cells such as lung cancer cells. Preferably, the term “brain cancer” refers to primary brain tumors.

Primary brain tumors are categorized by the type of tissue in which they first develop. The most common brain tumors are called glioma; they originate in the glial tissue. There are a number of different types of gliomas: for instance, astrocytomas, brain stem gliomas, ependymomas, and oligodendrogliomas.

Other types of primary brain tumors which do not originate from the glial tissue are, for instance, meningiomas, craniopharyngiomas and germinomas.

Treating lung and/or brain cancer can result in a reduction in size or volume of a tumor. A reduction in size or volume of a tumor may also be referred to as “tumor regression.” Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor or by any reproducible means of measurement.

Treating lung and/or brain cancer may further result in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating lung and/or brain cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. A metastasis is a region of cancer cells, distinct from the primary tumor location resulting from the dissemination of cancer cells from the primary tumor to other parts of the body. The number of metastatic lesions may be measured by any reproducible means of measurement. The number of metastatic lesions may be measured by counting metastatic lesions visible to the naked eye or at a specified magnification. Preferably, the specified magnification is 2×, 10×, or 50×.

Treating lung and/or brain cancer can result in an increase in average survival time of a population of subjects treated according to the present invention in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with the compound of Formula I. An increase in average survival time of a population may also be measured, for example, by calculating for a population the average length of survival following completion of a first round of treatment with the compound of Formula I.

Treating lung and/or brain cancer can also result in a decrease in the mortality rate of a population of treated subjects in comparison to an untreated population. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means, for example, by calculating for a population the average number of disease-related deaths per unit time following initiation of treatment with the compound of Formula I. A decrease in the mortality rate of a population may also be measured, for example, by calculating for a population the average number of disease-related deaths per unit time following completion of a first round of treatment with the compound of Formula I.

Another embodiment of the present invention relates to a method for preventing cancer by means of administering the compound of Formula I or a pharmaceutical composition thereof. Accordingly, treatment of an individual with the compound of Formula I or a pharmaceutical composition thereof reduces the risk of the individual to develop cancer. Preferably, after the treatment, the risk of the individual to develop cancer is reduced by 5% or greater; more preferably, the risk develop cancer is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. As used herein, reducing risk of developing cancer includes decreasing the probability or incidence of developing cancer for an individual compared to a relevant, e.g. untreated, control population, or in the same individual prior to treatment according to the invention. Reduced risk of developing cancer may include delaying or preventing the onset of a cancer. Risk of developing cancer can also be reduced if the severity of a cancer or a precancerous condition is reduced to such a level such that it is not of clinical relevance. That is, the cancer or a precancerous condition may be present but at a level that does not endanger the life, activities, and/or well-being of the individual. For example, a small tumor may regress and disappear, or remain static. Preferably, tumor formation does not occur. In some circumstances the occurrence of the cancer or the precancerous condition is reduced to the extent that the individual does not present any signs of the cancer or the precancerous condition during and/or after the treatment period.

The method for preventing cancer according to the present invention is beneficial both for individuals having a precancerous condition and individuals who are healthy. Individuals with lifestyle habits that could lead to cancer, particularly smokers, and individuals affected by diseases for which the probability of cancer incidence is high have a particularly high order of priority as individuals for the preventive method of the present invention. Furthermore, individuals who are likely to acquire familial cancers, and such individuals as those who are diagnosed with a risk of cancer by means of gene diagnoses based on single-nucleotide polymorphism or the like may also be targeted.

The compounds represented by Formula I and pharmaceutical compositions thereof have anticancer activity. Thus, the compounds represented by Formula I and pharmaceutical compositions thereof inhibit the growth of human or animal cancer cell lines such as A549 human lung cancer cells in in vitro tests and have IC₅₀ value of preferably less than 600 μM, more preferred of less than 100 μM, particularly preferred of less than 70 μM. The tests are preferably carried out as specified in S. Joseph et al. (Molecular Medicine Reports 2011, 4:891-899).

Some embodiments of the present invention are directed to the compound of Formula I and pharmaceutical compositions thereof for prevention and/or treatment of precancerous conditions of the lung. The term “precancerous conditions in the lung” as used therein refers to a group of cell proliferative disorders of the lung.

Cell proliferative disorders of the lung include all forms of cell proliferative disorders affecting lung cells. Cell proliferative disorders of the lung can include lung cancer, precancerous conditions of the lung. Cell proliferative disorders of the lung can include hyperplasia, metaplasia, and dysplasia of the lung. Cell proliferative disorders of the lung can include asbestos-induced hyperplasia, squamous metaplasia, and benign reactive mesothelial metaplasia. Cell proliferative disorders of the lung can include replacement of columnar epithelium with stratified squamous epithelium, precancerous lung lesion and mucosal dysplasia. Individuals exposed to inhaled injurious environmental agents such as cigarette smoke and asbestos may be at increased risk for developing cell proliferative disorders of the lung. Prior lung diseases that may predispose individuals to development of cell proliferative disorders of the lung can include chronic interstitial lung disease, necrotizing pulmonary disease, scleroderma, rheumatoid disease, sarcoidosis, interstitial pneumonitis, tuberculosis, repeated pneumonias, idiopathic pulmonary fibrosis, granulomata, asbestosis, fibrosing alveolitis, emphysema, and Hodgkin's disease.

The compounds represented by Formula I and pharmaceutical compositions thereof are further directed at individuals at risk of developing lung cancer. Such risk may be based on the medical or social history of an individual, such as inhalation of tobacco products as it occurs for example in smokers or exposure to asbestos or in non-smokers who breathe in secondhand smoke. Another category of individuals at risk for lung cancer are those harboring genetic mutations predisposing them to lung cancer. Yet another category is individuals who have been exposed to ionizing radiation or chemotherapeutic agents. Yet, another category is individuals with a known cancer at a location other than the lungs that have a propensity to metastasize to the lungs.

Finally, another category is individuals with prior lung cancer that has already been treated. Accordingly, the corresponding embodiment of the present invention relates to a method for preventing cancer recurrence by means of administering the compound of Formula I or a pharmaceutical composition thereof. Cancer recurrence is a re-development of the cancer in an individual, who had previously undergone a cancer treatment, after a period of time in which no cancer could be detected. The probability of a cancer recurring depend on many factors, including the type of cancer and its extent within the body at the time of the treatment.

The compounds of the present invention have high in vivo stability. Preferably, the concentration the compound of Formula I in blood plasma of an animal after 3 hr of administration is at least 30% of its initial concentration, more preferred at least 40% of its initial concentration, and particularly preferred at least 50% of its initial concentration. The corresponding tests can be carried out with animals such as mice according to the method described by Xie et al. (Xie G, Nie T, Mackenzie G, Sun Y, Huang L, Ouyang N, et al. Br. J. Pharmacol. 2011).

In addition, the compounds of the present invention have cellular uptake values, which can be determined by using cancer cells, for instance human non-small cell lung cancer cells A549 and subsequently assaying their intracellular levels by HPLC. The tests can be performed according to the method outlined in Example 2. Preferably, the cellular uptake values of the compounds are higher than 0.1 nmol/mg protein, more preferred higher than 1.0 nmol/mg protein, even more preferred higher than 10.0 nmol/mg protein and particularly preferred higher than 50.0 nmol/mg protein.

In one embodiment, the compounds of Formula I have n-octanol-water partition coefficient (log P) value higher than 2, more preferred higher than 3 and particularly preferred higher than 4. Log P is defined as ratio of concentrations (mol/volume) of the compounds of Formula I in n-octanol and in water. Suitable methods for the measurement of n-octanol-water coefficients are, for instance described in Octanol-Water Partition Coefficients: Fundamentals and Physical Chemistry, John Wiley and Sons Ltd., 1997, ISBN: 0-417-97397 1. Both solvents are mutually saturated before the measurement. At equilibrium the n-octanol phase contains 2.3 mol/l of water and the aqueous phase contains 4.5×10⁻³ mol/l of n-octanol. The measurement is carried out at the isoelectric point of the compound of Formula I at temperature of 25° C. The log P of the compounds of Formula I is preferably determined by the shake-flask method, which is, for example, described in the review of J. Sangster (J. Phys. Chem. Ref. Data 18, 1989; 3:1111-1227). The measurement is carried out under the conditions described by T. Fujita et al. (J. Am. Chem. Soc. 1964; 86:5175-5180) and the concentration of the compound of Formula I in each of the two phases is determined by high performance liquid chromatography (H PLC).

In a further aspect, the invention is directed to a pharmaceutical composition comprising a compound of Formula I, as described generally herein, and a pharmaceutically acceptable excipient.

In one embodiment, the pharmaceutical composition containing the compound of Formula I is used in the treatment and/or prevention of lung and/or brain cancer.

In yet another embodiment, the pharmaceutical composition containing the compound of Formula I is used in the treatment and/or prevention of precancerous conditions of the lung.

Pharmaceutical compositions of the present invention can comprise one or more other pharmaceutical agents in addition to one or more compounds of the invention.

In a specific embodiment, the invention is directed to a method for obtaining a pharmaceutical composition, comprising formulating the compounds of the present invention into a composition comprising the compounds and one or more pharmaceutically acceptable carriers or excipients. The invention is further directed to uses of the compounds of the present invention for manufacturing said pharmaceutical composition.

### Compositions

As discussed above, this invention provides novel compounds for use in the treatment and prevention of lung and/or brain cancer and precancerous conditions thereof, wherein said compounds are administered to a human or animal by the respiratory route. The term “respiratory route” as used herein refers to both nasal and pulmonary respiratory routes.

Administration by the nasal respiratory route includes nasal administration, and nose to brain delivery whereby the composition of the present invention is sprayed into the nasal cavity and delivered to the brain via the olfactory and trigeminal neural pathways. Nasal drug delivery is known to a person skilled in the art and is, for instance, described in L. Ilium (J. Control. Release 87 (2003), pp. 187-198). Administration by nasal respiratory route and nose to brain delivery is particularly suitable for the treatment of brain cancer and the corresponding precancerous conditions.

Preferably, the permeability of the nasal mucosa to the compounds of Formula I is high, and subsequently, their bioavailability upon nasal administration is more than 60%, preferably more than 70% and even more preferred more than 80%.

When the composition of the present invention is administered by the nasal respiratory route, more than 50 wt.-%, preferably more than 60 wt.-% and particularly preferred more than 70 wt.-% of the compound of Formula I is absorbed through the nasal mucosa and enters the systemic circulation of the patient. Thus, this embodiment of the present invention allows a rapid and effective administration of the compound of Formula I. Furthermore, if the aerosol particles have mass median aerodynamic diameter of less than 10 μm, up to 40 wt.-%, preferably up to 50 wt.-% and more preferred up to 60 wt.-% of the compound of Formula I is delivered to the lungs of the patient. Accordingly, the compound of Formula I is delivered to the lung cancer of the patient both locally and systemically.

The composition for nasal administration may be an aqueous solution designed to be administered to the nasal passages in form of drops or sprays. Preferably, this composition is isotonic to nasal secretions and slightly buffered to maintain a pH of 5.5 to 6.5. Antimicrobial agents and/or preservatives may be also present in this composition.

In another embodiment of the invention, the composition is administered by the oral respiratory route.

For administration by the respiratory route, the compounds can be delivered in the form of an aerosol spray from a pressurized container or dispenser, which contains a suitable propellant, e.g. hydrofluoroalkanes, chlorofluorocarbons, carbon dioxide, or a nebulizer. In the case of a pressurized aerosol, the dosage unit may be determined by providing a valve to deliver a metered amount. Capsules and cartridges of e.g. gelatine for use in an inhaler or insufflator may be formulated containing a powder mix of the compound and a suitable pharmaceutically acceptable carrier.

Administration by the respiratory route usually requires the use of pharmaceutical compositions suitable for the dispensing of the compounds of Formula I. Typically, each pharmaceutical composition is specific to the type of device employed and may involve the use of an appropriate propellant material, in addition to the usual diluents, adjuvants and/or carriers. Also, the use of liposomes, microcapsules or microspheres, inclusion complexes, or other types of carriers is contemplated. The compounds of Formula I may be prepared in different pharmaceutical compositions depending on their physical and chemical properties or the type of device employed.

Pharmaceutical composition suitable for use with a nebulizer, either jet or ultrasonic, will typically comprise the compound of Formula I dissolved in a solvent at a concentration of about 0.1 to 25 mg of the compound of Formula I per 1 ml of solution. The pharmaceutical composition may also include a buffer, for instance, an aminoacid, and a simple sugar (e.g. for compound of Formula I stabilization and regulation of osmotic pressure). The solvent in the pharmaceutical composition may be selected from the group consisting of water, ethanol, 1,3-propylene glycol, glycerol or a mixture of any of those. Nebulized pharmaceutical compositions may also contain a surfactant, to reduce or prevent surface induced aggregation of the compound of Formula I caused by atomization of the solution in forming the aerosol.

Pharmaceutical compositions for use with a metered-dose inhaler device generally comprise a finely divided powder containing the compound of Formula I (or a pharmaceutically acceptable derivative thereof) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Pharmaceutical compositions for dispensing from a powder inhaler device will comprise a finely divided dry powder containing the compound of Formula I and may also include a bulking agent, such as lactose, sorbitol, sucrose, or mannitol in amounts, which facilitate dispersal of the powder from the device, e.g. 50 to 90% by weight of the formulation. The compound of Formula I should most advantageously be prepared in a particulate form with an average particle size of less than 10 μm, preferably less than 5 μm and more preferred less than 1 μm, for effective delivery to the distal lung.

In another aspect of the present invention, pharmaceutical compositions are provided, which comprise the compound of Formula I (or a pharmaceutically acceptable salt, cocrystal or other pharmaceutically acceptable derivative thereof), and optionally comprise a pharmaceutically acceptable carrier. In certain embodiments, these compositions optionally further comprise one or more additional therapeutic agents. Alternatively, the compounds of this invention may be administered to a patient in need thereof in combination with the administration of one or more other therapeutic agents. For example, additional co-administered therapeutic agents or included in a pharmaceutical composition with a compound of this invention may be an approved anti-inflammation or analgesic agent, or it may be any one of a number of agents undergoing approval in the Food and Drug Administration that ultimately obtain approval for the treatment of any disorder related to inflammation and pain. Such additional therapeutic agents may also be provided to promote the targeting of the compounds of the invention to the desired site of treatment, or may increase their stability, increase their plasma half-life, and further improve their biodistribution and pharmacokinetics. It will also be appreciated that certain of the compounds of present invention can exist in a free form for treatment, or where appropriate, as a pharmaceutically acceptable derivative thereof. According to the present invention, a pharmaceutically acceptable derivative includes, but is not limited to, pharmaceutically acceptable salts, esters, salts or cocrystals of such esters, or a pro-drug or other adduct or derivative of a compound of this invention which upon administration to a patient in need is capable of providing, directly or indirectly, a compound as otherwise described herein, or a metabolite or residue thereof.

As used herein, the term “pharmaceutically acceptable salt or cocrystals” refers to those salts or cocrystals which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts or cocrystals of amines, carboxylic acids, and other types of compounds, are well known in the art. For example, S. M. Berge et al. describe pharmaceutically acceptable salts in detail in J. Pharmaceutical Sciences, 66: 1-19 (1977), incorporated herein by reference. The salts can be prepared in situ during the final isolation and purification of the compounds of the invention, or separately by reacting a free base or free acid function with a suitable reagent, as described generally below. For example, a free base function can be reacted with a suitable acid. Furthermore, where the compounds of the invention carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino substituent formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts and coformer molecules for cocrystal formation, include adipate, alginate, ascorbate, aspartate, benzenesulfonate, benzoate, bisulfate, borate, butyrate, camphorate, camphorsulfonate: citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound or a salt thereof. Suitable ester substituents include, for example, those derived from pharmaceutically acceptable aliphatic carboxylic acids, particularly alkenoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are transformed in vivo to yield the parent compound of the above formula, for example, by hydrolysis in blood. A thorough discussion is provided in T. Higuchi and V. Stella, Prodrugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

As described above, the pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to volatile solid materials, such as menthol, sugars such as lactose, glucose and sucrose; excipients such as cocoa butter; oils such as peanut oil, cottonseed oil; safflower oil, sesame oil; olive oil; corn oil and soybean oil; glycols; such as propylene glycol; esters such as ethyl oleate and ethyl laurate; agar; natural and synthetic phospholipids, such as soybean and egg yolk phosphatides, lecithin, hydrogenated soy lecithin, dimyristoyl lecithin, dipalmitoyl lecithin, distearoyl lecithin, dioleoyl lecithin, hydroxylated lecithin, lysophosphatidylcholine, cardiolipin, sphingomyelin, phosphatidylcholine, phosphatidyl ethanolamine, diastearoyl phosphatidylethanolamine (DSPE) and its pegylated esters, such as DSPE-PEG750 and, DSPE-PEG2000, phosphatidic acid, phosphatidyl glycerol and phosphatidyl serine. Commercial grades of lecithin which are preferred include those which are available under the trade name Phosal® or Phospholipon® and include Phosal® 53 MCT, Phosal® 50 PG, Phosal® 75 SA, Phospholipon® 90H, Phospholipon® 90G and Phospholipon® 90 NG; soy-phosphatidylcholine (SoyPC) and DSPE-PEG2000 are particularly preferred; buffering agents such as amino acids; pyrogen-free water; isotonic saline; Ringer's solution; ethyl alcohol, and phosphate buffer solutions, as well as other non-toxic compatible lubricants such as sodium lauryl sulfate as well as releasing agents, coating agents, sweetening, flavoring and perfuming agents, preservatives and antioxidants can also be present in the pharmaceutical composition, according to the judgment of the formulator.

The compounds represented by Formula I are also suitable for incorporation into nanoparticulate systems such as liposomes, polymeric nanoparticles, polymeric micelles, lipid nanoparticles, micro- and nano-emulsions, nanogels, liposomes being particularly preferred. The corresponding nanoparticulate systems are known in the prior art and are, for instance, described in the review by Wu and Mansour (X. Wu and H. M Mansour, Invited Paper. International Journal of Nanotechnology: Special Issue-Nanopharmaceuticals, 2011, 8, 1/2, 115-145).

Nanoparticulate systems typically have an average particle size ranging from 1 to 1000 nm, preferably from 50 to 500 nm. The term “liposomes” as used herein refers to phospholipid vesicles with average particle size ranging from 50 to 1000 nm, which are formed by one or several lipid bilayers with an aqueous phase both inside and between the bilayers. The term “polymeric nanoparticles” refers to solid colloidal particles comprising polymeric materials. The average particle size of polymeric nanoparticles ranges from 30 to 300 nm.

Polymeric micelles are particles formed through the self-assembly of amphiphilic block copolymers containing hydrophobic and hydrophilic blocks.

Lipid nanoparticles may be in the form of solid lipid nanoparticles, nanostructured lipid carriers or lipid drug conjugates. Microemulsions are typically characterized by the average internal globule size of less than 150 nm. Microemulsions require a surfactant concentration of at least 10 wt.-%, preferably of at least 50 wt.-% and more preferred of at least 20 wt.-%, based on the weight of the composition.

The term “nanogel” refers to aqueous dispersions of hydrogel particles formed by physically or chemically cross-linked polymer networks of nanoscale size. Nanogels can be prepared by a variety of methods such as self-assembly of polymers, polymerization of monomers, cross-linking of preformed polymers or template-assisted nanofabrication.

Use of nanoparticulate systems according to the present invention provides sustained-release of the compound of Formula I in the lung tissue, resulting in a reduction of dosing frequency and improved patient compliance and further enabling uniformity of drug dose distribution among the alveoli. Moreover, by formulating the compounds of Formula I as in nanoparticulate systems, one can achieve a dose that is higher than that of other pharmaceutical compositions, which are limited by the solubility volatibility of the compound of Formula I. Nanoparticles can be internalised by a variety of cell types and becide macrophages, other cells like cancer cells and epithelial cells are also able to take up nanoparticles. Therefore, usage of nanoparticulate systems for delivering the compounds of Formula I is highly advantageous for the treatment and prevention of lung cancer.

Nanoparticulate formulations can further be advantageously used for the nasal delivery of the compounds represented by Formula I. In this embodiment, multiple-unit mucoadhesive nanoparticles are preferably used in order to prolong the contact of the compound of Formula I with the nasal mucosa.

The resulting compositions can be advantageously employed for administration by the respiratory route. Preferred liposome compositions are those which in addition to other phospholipids, incorporate pegylated phospholipids, such as DSPE-PEG2000, and exhibit long circulation times by avoiding uptake and clearance by the reticuloendothelial system (RES) and thus, are able to reach and treat lung cancer tumors.

In some embodiments, the pharmaceutical composition may further comprise an additional compound having anticancer activity. The additional compound having anticancer activity can be selected from the group of compounds such as chemotherapeutic and cytotoxic agents, differentiation-inducing agents (e.g. retinoic acid, vitamin D, cytokines), hormonal agents, immunological agents and anti-angiogenic agents. Chemotherapeutic and cytotoxic agents include, but are not limited to, alkylating agents, cytotoxic antibiotics, antimetabolites, vinca alkaloids, etoposides, and others (e.g., paclitaxel, taxol, docetaxel, taxotere, cis-platinum). A list of additional compounds having anticancer activity can be found in L. Brunton, B. Chabner and B. Knollman (eds). Goodman and Gilman's The Pharmacological Basis of Therapeutics, Twelfth Edition, 2011, McGraw Hill Companies, New York, N.Y.

In a preferred embodiment, the additional compound having anticancer activity is a tyrosine kinase inhibitor (TKI). A TKI inhibits the tyrosine kinase activity of at least one tyrosine kinase. The inhibition may be reversible or irreversible. TKIs include, but are not limited to, agents such as imatinib, dasatinib, nilotinib, gefitinib, erlotinib, lapatinib, sunitinib, sorafenib and pazopanib. Various TKIs are, for instance, described in Hartmann et al. (J. Th. Hartman et al. Cur. Drug Metab, 2009, 10, pp. 470-481).

In another embodiment, the additional compound having anticancer activity is a compound with oxidative stress-inducing ability. These compounds increase the oxidative stress of cancer cells by inhibiting the mechanisms that cancer cells utilize to compensate for reactive oxygen species (ROS) and/or activating cellular signaling pathways that lead to immunocytotoxicity. Examples of the anticancer drug include platinum formulation such as cis-platin, carboplatin, and oxaliplatin, thiostrepton, cyclophosphamide, fluorouracil, etoposide, doxorubicin, bleomycin, and mitomycin. The term “reactive oxygen species” relates to highly reactive metabolites of molecular oxygen, which are generated in a tissue environment. ROS can be free radicals, ions or molecules. Examples of ROS include, but are not limited to, superoxide ion radical (O₂⁻), hydroxyl radical (OH.), peroxide (ROO.), alkoxyl radicals (RO.), hydrogen peroxide (H₂O₂), organic peroxide (ROOR′), ozone (O₃), singlet oxygen (¹O₂), etc.

Additional compounds having anticancer activity are preferably difluoromethylornithine, erlotinide and thiostrepton.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with an anti-inflammation or anticancer agent), or they may achieve different effects (e.g. control of any adverse effects).

In certain embodiments, the pharmaceutical compositions of the present invention further comprise one or more additional therapeutically active ingredients (e.g. anti-inflammatory and/or palliative). For purposes of the invention, the term “palliative” refers to treatment that is focused on the relief of symptoms of a disease and/or side effects of a therapeutic regimen, but is not curative. For example, palliative treatment encompasses painkillers, antinausea medications and anti-sickness drugs.

### Uses and Methods of Treatment

As discussed above, the compounds of Formula I as well as pharmaceutical compositions comprising these compounds are useful for the treatment and/or prevention of lung cancer and precancerous condition of the lung.

The compounds of Formula I as well as pharmaceutical compositions thereof may be administered by the nasal or oral respiratory route. For example, the compounds of Formula I and pharmaceutical compositions thereof can be suspended or dissolved in an appropriate carrier and administered directly into the lungs using a nasal spray or inhalant.

Alternatively, the compounds of Formula I and pharmaceutical compositions thereof may be sprayed into the nasal cavity and absorbed through the nasal mucosa.

Importantly, direct inhalational administration of compounds of Formula I and pharmaceutical compositions thereof into the lungs provides several advantages over oral administration:
a) A lower amount of the compound of Formula I is required for achieving the same therapeutic effect. This may be critical for expensive compounds.
b) Any potential undesired side effects of the compound of Formula I are minimized.
c) Inactivation of the compound of Formula I in vivo through first-pass metabolism, e.g. by non-specific esterases in the intestine and liver is substantially avoided.
d) Improved absorption using aerosol drug delivery.

Accordingly, in another aspect of the invention, methods for the treatment and/or prevention of lung and/or brain cancer and precancerous condition thereof are provided comprising administering a therapeutically effective amount of the compound of Formula I to a subject in need thereof by the respiratory route. In certain embodiments, the compounds of Formula I and pharmaceutical compositions comprising these compounds are administered in such amounts and for such time as is necessary to achieve the desired result.

The invention is also directed to the use of any compound of Formula I for the preparation of a medicament for administration to a human or animal patient in need thereof for the treatment and/or prevention of lung and/or brain cancer and precancerous condition thereof. Such compounds are preferably administered once a precancerous condition of the lung or lung and/or brain cancer has been diagnosed in the patient, optionally in combination with anti-inflammation agents or other anticancer agents such as those that maintain therapeutic levels of the compounds within the body. Compounds of the invention also may be administered after other therapies have failed.

In another embodiment, the compounds of Formula I as well as pharmaceutical compositions comprising these compounds may be administered prophylactically for the purpose of prevention of lung and/or brain cancer. Thus, the compounds of Formula I and the pharmaceutical compositions comprising these compounds may be administered to subjects having an increased risk of developing lung and/or brain cancer, for instance to smokers. Lung cancer is the culmination of a long transition of the tracheal epithelium from normal through various precancerous stages. Thus, administration of the compounds of Formula I before or during this transitional period is simpler for the patient and is further cost-effective compared to current therapeutic modalities for already developed lung cancer.

In some embodiments of the invention, the pharmaceutical composition is administered to a human or animal in the form of an aerosol.

Yet in other embodiments of the invention, the pharmaceutical composition is administered to a human or animal in the form of a dry powder aerosol.

In a further embodiment of the invention the pharmaceutical composition is administered to a human or animal, preferably to a human, in combination with tobacco smoke. Thus, the corresponding envisioned mode of administration is for the compound of Formula I to be inhaled at the same time when the smoker smokes.

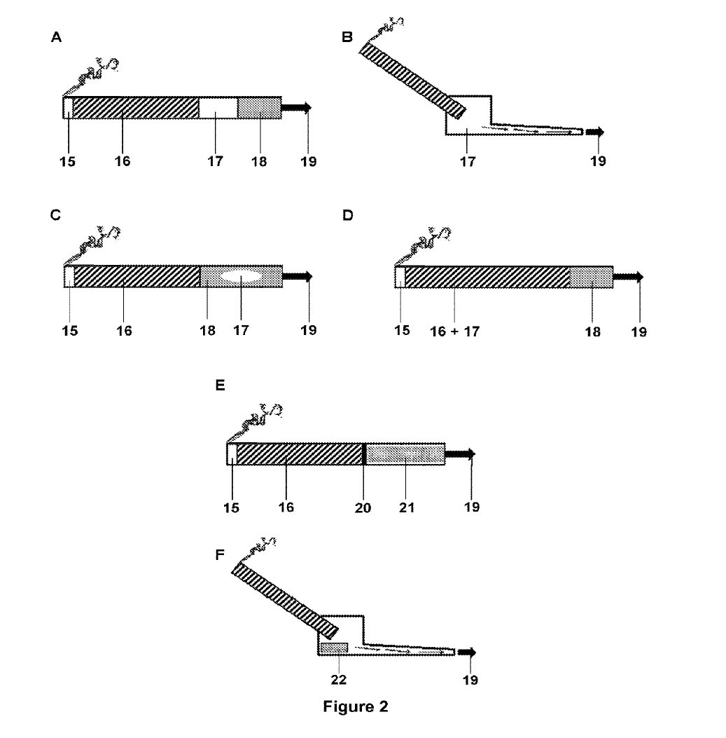

For this purpose, the compound of Formula I or a pharmaceutical composition thereof can be incorporated in a smoking device such as for instance a cigarette or a smoking pipe as shown in FIG. 2. In the embodiments illustrated by FIGS. 2A-2D, the number 17 indicates the location of the compound of Formula I or a pharmaceutical composition thereof. In the smoking devices shown in FIG. 2E and FIG. 2F the compound of Formula I or a pharmaceutical composition thereof is located in the cartridge 21 and in the additional unit 22 respectively.

In some embodiments of the invention, the compound of Formula I or a pharmaceutical composition thereof can be directly mixed with tobacco. In these embodiments, a vaporization of the compound of Formula I takes place in the pyrolysis zone of the smoking device. These embodiments are particularly preferred for sufficiently volatile compounds of Formula I. The vaporization of the compound of Formula I can be additionally facilitated, when volatile solids such as menthol are used as carriers in the pharmaceutical composition.

The term “tobacco” as used herein relates to the leaf of a tobacco plant i.e. a plant of the genus Nicotiana, such as Nicotiana tabaccum. Tobacco leaves of several types may be employed. Suitable types of tobacco leaves include, but are not limited to, Brightleaf tobacco, Burley, Cavendish, Corojo, Criollo, Oriental tobacco, Perique, Shade tobacco, Thuoc Iao, Type 22, White Burley, wild tobacco and Yl.

In other embodiments of the invention, the pharmaceutical composition comprising the compound of Formula I is spatially separated from the tobacco.

Preferably, the aerosol particles comprise less than 10 wt.-% of degradation products formed by the compound of Formula I. More preferred, the particles comprise less than 5 wt.-% of degradation products formed by the compound of Formula I. Yet even more preferred, the particles comprise less than 1.0 wt.-%, for instance less than 0.5 wt.-% of degradation products formed by the compound of Formula I.

Preferably, at least 50 wt.-% of the aerosol is amorphous in form, wherein crystalline forms make up less than 50 wt.-% of the total aerosol weight, regardless of the nature of individual particles. More preferred, at least 75 wt.-% of the aerosol is amorphous in form. Particularly preferred, at least 90 wt.-% the aerosol is amorphous in form.

Typically, the aerosol has an inhalable aerosol particle density greater than 10⁶ particles/ml. Preferably, the aerosol has an inhalable aerosol particle density greater than 10⁷ particles/ml or 10⁸ particles/ml.

Preferably, the aerosol particles have a mass median aerodynamic diameter of between 3 μm and 0.02 μm, more preferred between 2 μm and 0.05 μm, even more preferred between 1 μm and 0.1 μm, particularly preferred between 0.8 μm and 0.2 μm.

Particle size distribution of the aerosol can be determined using any suitable method in the art (e.g. cascade impaction). For example, an Andersen Eight Stage Nonviable Cascade Impactor (Andersen Instruments, Smyrna, Ga.) linked to a furnace tube by a mock throat (USP throat, Andersen Instruments, Smyrna, Ga.) is one system used for cascade impaction studies.

Inhalable aerosol mass density is determined, for example, by delivering a drug-containing aerosol into a confined chamber via an inhalation device and measuring the mass collected in the chamber. Typically, the aerosol is drawn into the chamber by having a pressure gradient between the device and the chamber, wherein the chamber is at lower pressure than the device. The volume of the chamber should approximate the tidal volume of an inhaling patient.

Inhalable aerosol drug mass density is determined, for example, by delivering a drug-containing aerosol into a confined chamber via an inhalation device and measuring the amount of active drug compound collected in the chamber. Typically, the aerosol is drawn into the chamber by having a pressure gradient between the device and the chamber, wherein the chamber is at lower pressure than the device. The volume of the chamber should approximate the tidal volume of an inhaling patient. The amount of the compound of Formula I collected in the chamber is determined by extracting the chamber, conducting chromatographic analysis of the extract, for instance by using analytical HPLC, and comparing the results of the chromatographic analysis to those of a standard containing known amounts of the compound of Formula I.

The expression “effective amount” as used herein, refers to a sufficient amount of the compound of Formula I to exhibit the desired therapeutic effect. The exact amount required will vary from subject to subject, depending on the species, age, and general condition of the subject, the particular therapeutic agent and the like. The compounds of the invention are preferably formulated in dosage unit form for ease of administration and uniformity of dosage. The expression “dosage unit form” as used herein refers to a physically discrete unit of therapeutic agent appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the anticancer activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Furthermore, after pharmaceutical composition with appropriate pharmaceutically acceptable carriers in a desired dosage form, the pharmaceutical compositions of this invention can be administered to a human or animal subject. In certain embodiments, the compounds of the invention may be administered by inhalation at dosage levels of 0.001 mg/kg to 50 mg/kg, from 0.01 mg/kg to 25 mg/kg or from 0.1 mg/kg to 10 mg/kg of subject body weight per day, one or more times a day, to obtain the desired therapeutic effect. In other embodiments, compounds of the invention may be administered at dosage levels of 0.01 mg/kg to 100 mg/kg, from 0.05 mg/kg to 50 mg/kg or from 0.1 mg/kg to 10 mg/kg of subject body weight per day, one or more times a day, to obtain the desired therapeutic effect. It will also be appreciated that dosages smaller than 0.001 mg/kg or greater than 50 mg/kg (for example 50 mg/kg to 100 mg/kg) can be administered to a subject.

The inhalation of the compound of Formula I can take place between one and seven times a day, for instance three times a day.

### Inhalation Devices

Contemplated for use in the practice of this invention are a wide range of mechanical devices designed for pulmonary delivery of the pharmaceutical compositions of the present invention, including but not limited to nebulizers, metered dose inhalers, and powder inhalers, all of which are familiar to those skilled in the art. Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp, Bedford, Mass.

Device for the nasal drug delivery are also known to persons skilled in the art and are commercially available, for instance, from Bespak (Bespak Europe Limited, United Kingdom).

In some other embodiments, the pharmaceutical composition of the present invention is directly heated, whereby the compound of Formula I forms a vapor and subsequently condenses into an aerosol. Thus, an aerosol containing the compound of Formula I is formed. Subsequently, the patient inhales this aerosol. Suitable devices are known in the prior art and are, for instance, described in US 2003/0000518.

In another embodiment, the compound of Formula I or the pharmaceutical composition is dissolved in a solvent such as ethanol, glycerol, water, 1,3-propylene glycol or in a mixture of any of those. For instance, ethanol can be employed for this purpose.

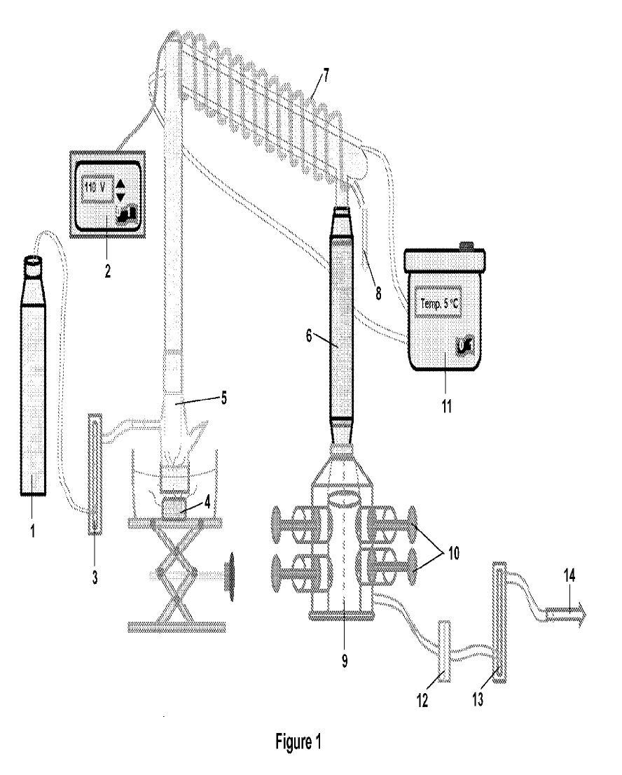

An example of inhalation device, in which the compound of Formula I is dissolved in a solvent is shown in FIG. 1. This exposure system can be employed for pre-clinical and clinical studies as well as for routine administration of the compound of Formula I to the patients. Air flow in the device is controlled by two major elements:
a) an inlet air regulator equipped with a flow meter 3, which pushes air 1 into the device via the baffle 5; and
b) a vacuum pump 14 which draws air from the system. The air entering the vacuum pump 14 passes through a filter 12 and a flow meter 13.

The compound of Formula I is dissolved in ethanol and the solution in the baffle 5 is aerosolized with the ultrasonic atomizer 4. The aerosol formed passes through an ascending stainless steel column, followed by a reflux column which is maintained at a temperature gradient by a heating tape 7 (82°) and a condenser (5° C.) to condense and remove ethanol. The temperature of the heating tape 7 is adjusted by the voltage regulator 2. The aerosol of the compound of Formula I exiting the reflux column then passes through a charcoal column 6 which serves to remove residual traces of ethanol from the aerosol before it enters the chamber 9. The patient can inhale the aerosol from air-tight tubes 10 for desired time intervals.

In another embodiment, the compound of Formula I is administered in a so-called electronic cigarette. Such devices are known in the prior art and are, for instance, described in US 2006/0196518, US 2007/0267031 and Caponnetto et al (Journal of Medical Case Reports 5, 585, 2011). An electronic cigarette is primarily used for the administration of nicotine and, optionally, of flavors such as menthol. Incorporating of the compound of Formula I or the corresponding pharmaceutical composition in the nicotine cartridge thus allows efficient administration of the compound of Formula I by the respiratory route. Advantageously, the cartridge comprising the compound of Formula I can be employed with a commercially available electronic cigarette.

Accordingly, another aspect of the present invention relates to a cartridge containing the compound of Formula I or the pharmaceutical composition thereof for use in an electronic cigarette. Such cartridge can be primarily used by patients suffering from lung cancer or those with precancerous conditions in the lung.

Another envisioned mode of administration is for the compound of Formula I to be inhaled at the same time that the smoker smokes. For this purpose, the compound of Formula I or the pharmaceutical composition thereof can be for instance incorporated in a cigarette, a cigar (see FIG. 2A) or in a smoking device such as a smoking pipe (see FIG. 2B in the chamber of a smoking pipe) or in a water pipe etc.

FIG. 2A: the pharmaceutical composition 17 containing the compound of Formula I is incorporated into the cigarette containing tobacco 16 and, optionally, having a filter 18. Tobacco smoke coming from the pyrolysis zone 15 causes volatilization of the compound 17. In order to improve volatilization the compound of Formula I can be formulated with a volatile solid such as menthol. The tobacco smoke 19 containing the compound of Formula I enters the mouth and the lungs of the smoker.

FIG. 2B: the pharmaceutical composition 17 is incorporated into a smoking pipe. Alternatively, another smoking device such as water pipe can be employed. The volatilization of the pharmaceutical composition 17 can be additionally facilitated by an external heating, for instance, by using an electric heating element.

In FIG. 2C a further embodiment of the present invention is shown. The compound of Formula I is administered in a so-called “cigarette with menthol capsules”. The pharmaceutical composition 17 is incorporated in a menthol capsule, which, in turn, is located in the filter 18. Cigarettes with menthol capsules are known in the prior art and are, for instance, described in US 2009/0277465. The compound of Formula I or the pharmaceutical composition thereof is incorporated into the menthol capsule and is volatilized during the smoking process. Thus, this embodiment is particularly suited for smokers and aims to prevent lung cancer and/or precancerous conditions in the lung.

In a further embodiment shown in FIG. 2D, the pharmaceutical composition 17 is directly mixed with tobacco. Thus, volatilization the compound of Formula I occurs primarily in the pyrolysis zone 15 of the cigarette and the tobacco smoke 19 containing the compound of Formula I enters the mouth and the lungs of the smoker. In this embodiment, the filter 18 is optional. This embodiment is particularly useful, if the compound of Formula I is sufficiently volatile.

A further embodiment is shown in FIG. 2E. The pharmaceutical composition 17 (not shown) is incorporated in an electronic cigarette cartridge 21. Valve 20 prevents the entry of the aerosol and solvent vapor emitted by the cartridge 21 into the tobacco section 16. In this embodiment, tobacco smoke formed in the pyrolysis zone 15 enters the section containing the electronic cigarette cartridge 21 via the valve 20. Thus, the aerosol emitted by the electronic cigarette cartridge 21 is mixed with the tobacco smoke and the resulting mixture 19 is subsequently inhaled by the smoker.

FIG. 2F: a further embodiment is shown. The anti-cancer agent or a pharmaceutical composition thereof 17 (not shown) is incorporated in an additional unit 22 which may be an atomizer or cartonizer or similar device that renders the anti-cancer agent suitable for inhalation. Having an appropriate valve or other mechanism(s), smoke and inhalable agent may be mixed to simultaneously deliver smoke and anti-cancer agent to the mouth and ultimately the lungs of the smoker.

In all embodiments shown in FIGS. 2A-2F during inhalation of tobacco smoke, the smoker also inhales the compound of Formula I. In order to facilitate volatilization of the compound of Formula I, it can be formulated in a dry powder aerosol composition such as the one described by C. Plumley, et at. (Int. J. Pharm. 369, (1-2), pages 136-143, 2009) or in a pharmaceutical composition containing volatile solids such as menthol. Alternatively, a neat compound of Formula I can be used instead of the pharmaceutical composition thereof.

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### EXAMPLES

### Materials and Methods

All reagents and solvents were ACS grade. All experiments involving moisture- or air-sensitive compounds were conducted under dry nitrogen. The starting materials and reagents, unless otherwise specified, were of the best grade commercially available (Aldrich, Fluka) and used without further purification. After purification, all new products showed a single spot on TLC analysis in two different solvent systems. All experiments were performed under atmospheric pressure of 100±5 kPa and room temperature unless stated otherwise. The term “room temperature” refers to a temperature of 20±2° C.

### Example 1

### Phosphoric acid diethyl ester 4-[2-(4-isobutyl-phenyl)-propionylamino]-butyl ester (phospho-ibuprofen amide, 1)

The title compound 1 was synthesized as shown in Scheme 1 below.

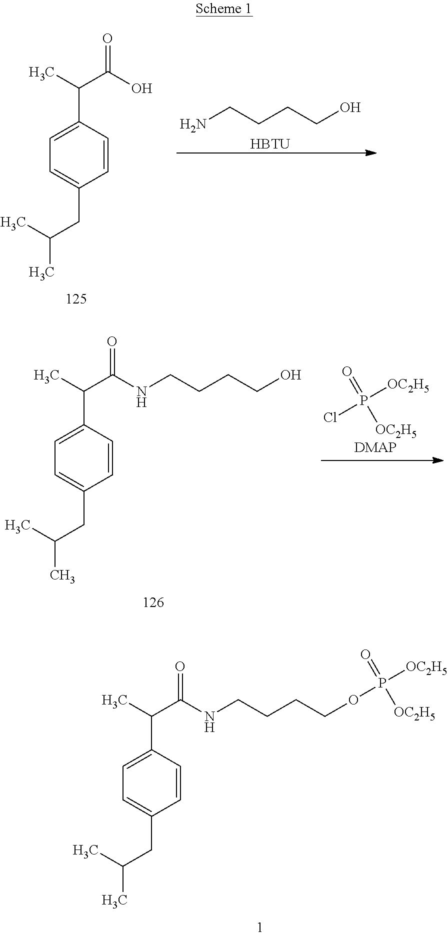

### Step 1.1 Synthesis of N-(4-Hydroxy-butyl)-2-(4-isobutyl-phenyl)-propionamide (126)

Ibuprofen (125) (0.228 g, 1 mmol), 4-amino-1-butanol (0.138 ml, 1.5 mmol) and 0-(Benzotriazol-1-yl)-N,N,N′,N′-tetramethyluronium hexafluorophosphate (HBTU) (0.57 g, 1.5 mmol) were dissolved in 5 ml of N,N-dimethylformamide (DMF) containing N,N-diisopropylethylamine (DIPEA) (0.17 ml, 1 mmol). The reaction mixture was stirred at room temperature for 4 h. The reaction was monitored by TLC. The resulting reaction mixture was dissolved in ethyl acetate, and then washed with 1M HCl, saturated aqueous NaHCO₃ solution, distilled water, brine and dried over sodium sulfate (Na₂SO₄). After the solvent was removed, the crude product was purified by flash column chromatography to give 126 as a white solid in 95% yield.

### Step 1.2 Synthesis of phosphoric acid diethyl ester 4-[2-(4-isobutyl-phenyl)-propionylamino]-butyl ester (1)

Under nitrogen, diethyl chlorophosphate (0.43 g, 1.25 mmol) was added drop-wise to a solution of alcohol 126 (0.299 g, 1 mmol) in dichloromethane (10 ml) containing DIPEA (0.17 ml, 1 mmol), and 4-(dimethylamino)pyridine (DMAP) (6 mg, 0.05 mmol). The reaction mixture was stirred overnight and monitored by TLC. The obtained reaction solution was washed with water (2×25 ml), dried over anhydrous Na₂SO₄, filtered and concentrated. The crude residue was purified by column chromatography using n-hexane:ethyl acetate (60:40) as eluent. The pure fractions were combined and evaporated to give a slightly yellow liquid 1 in 85% yield.

Biodistribution of Phospho-Ibuprofen Amide 1

Methods:

Phospho-ibuprofen amide 1 was formulated in liposomes following the standard protocols described by Mattheolabakis et al., (G. Mattheolabakis, T. Nie, P. P. Constantinides, B. Rigas, Pharm. Res. 2012; 29:1435-43) and administered intravenously to mice as a single 200 mg/kg i.v. dose. After 1 h, blood and all major organs were collected and drug concentration was determined in them following already published methods (T. Nie et al. Br J. Pharmacol. 2012; 166(3):991-1001).

Results:

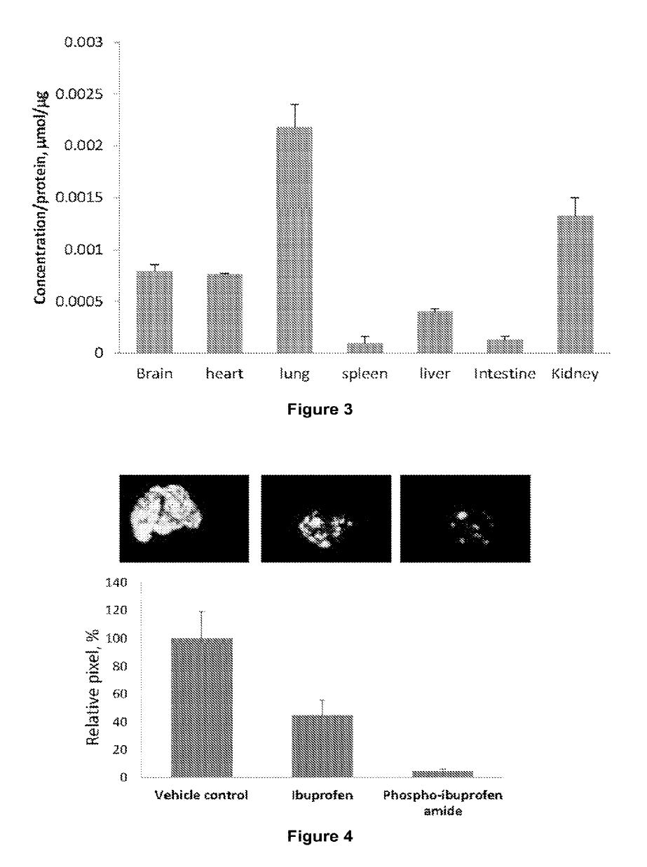

As shown in FIG. 3, liposomal phospho-ibuprofen amide 1 preferentially accumulated in lungs.

Efficacy of Phospho-Ibuprofen Amide 1: Inhibition of Lung Cancer

Methods: Female Ncr nude mice (6-7 weeks old) were injected i.v. (via their tail vein) with 6×10⁶ A549 human non-small lung cancer cells engineered to stably express green fluorescence protein. These cells were transplanted to the lungs (orthotopic lung tumor model). Three groups (n=6) of such mice were treated with a) liposomal phospho-ibuprofen amide 1 200 mg/kg, or b) ibuprofen (125) 200 mg/kg or c) vehicle once a week for 8 weeks. Mouse fluorescence was monitored using an in vivo imaging system (Maestro, Wobum, Mass.). Relative green fluorescence intensity units (from 7.5×10⁴ to 3.0×10⁵) were used as a marker for tumor initiation in the lungs. Day 0 was designated as initial detection of disease and the day before start of treatment. At the end of the study, animals were sacrificed and their tumors were removed, weighed and imaged.

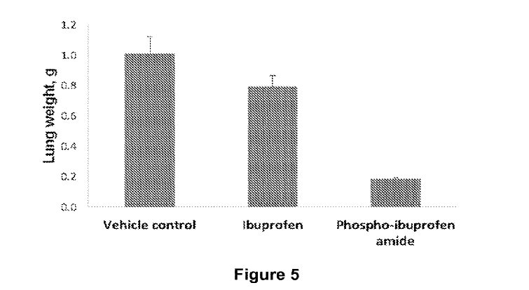

Results: FIG. 4 shows, in addition to representative fluorescence images of lungs from control (left), ibuprofen (center) and phospho-ibuprofen amide 1 (left) treated mice, the amount of lung tumor per group (based on fluorescence intensity). FIG. 5 depicts the lung weight of the same groups of animals. Values (% control) are mean±SEM.

Phospho-ibuprofen amide 1 essentially eliminated lung cancer, reducing it, by 95% based on fluorescence and by 80% base on lung tumor weight. In contrast, ibuprofen (125) reduced tumor fluorescence by 57% and lung weight by 19%. The differences between phospho-ibuprofen amide 1 and ibuprofen (125) were statistically significant (p<0.01). These findings underscore the efficacy of the compounds of the invention.

### Example 2

### [1-(4-Chloro-benzoyl)-5-methoxy-2-methyl-1H-indol-3-yl]-acetic acid 4-[2-(diethoxy-phosphoryloxy)-ethyl]-phenyl ester (phospho-tyrosol-indomethacin, 93)

The title compound 93 was synthesized as shown in Scheme 2 below:

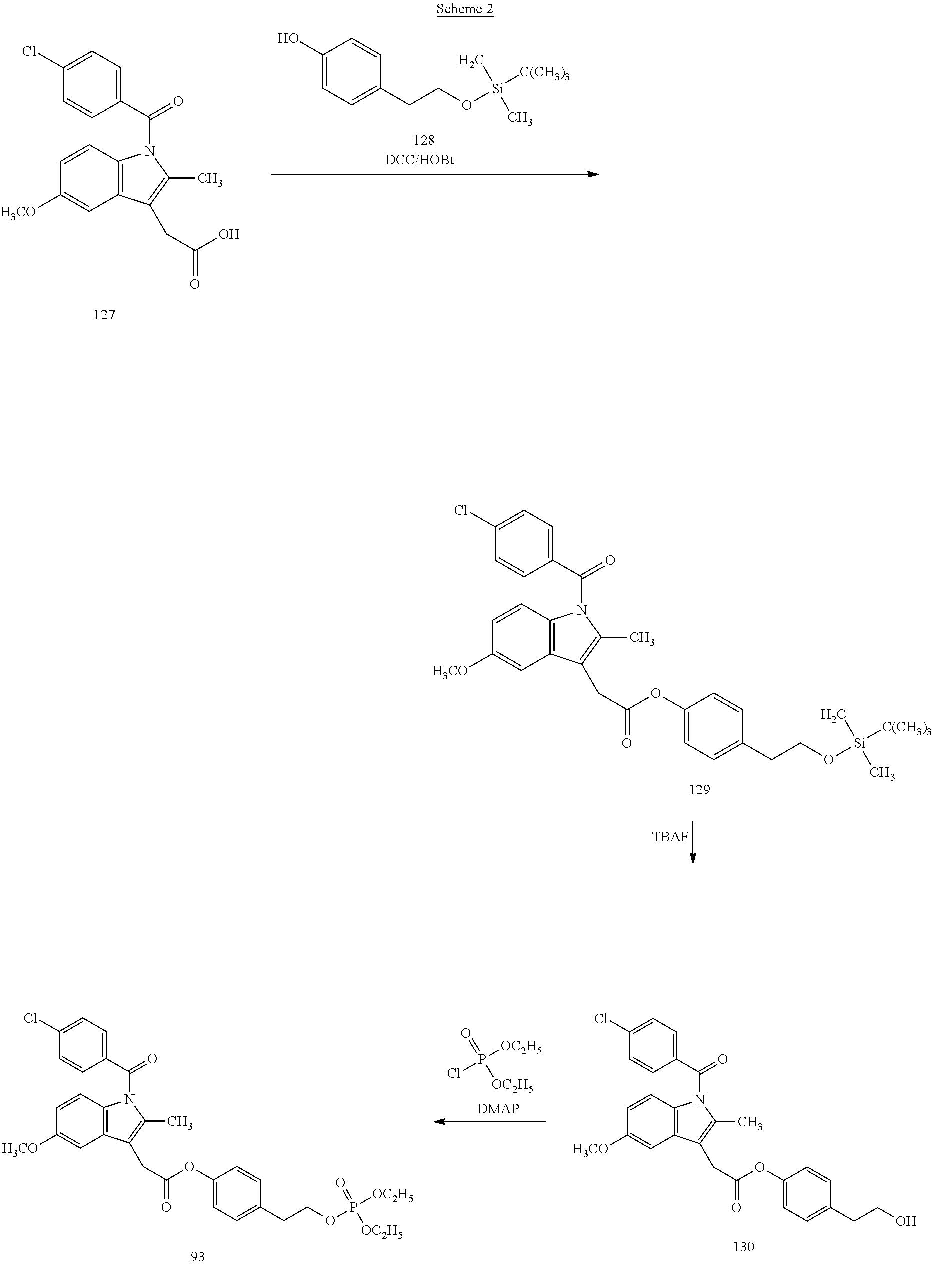

### Step 2.1 Synthesis of [1-(4-chloro-benzoyl)-5-methoxy-2-methyl-1H-indol-3-yl]-acetic acid 4-(2-hydroxy-ethyl)-phenyl ester (129)

Under nitrogen atmosphere, indomethacin (127) (1.0 g, 3 mmol), N,N′-dicyclohexylcarbodiimide (DCC) (0.9 g, 3.2 mmol), 1-hydroxybenzotriazole (HOBt) (0.6 g, 3 mmol) and dichloromethane (20 ml) were added to a flask and stirred at room temperature for 1 h. Then, a solution of the phenol 128 (0.9 g, 3.2 mmol) and DMAP (60 mg) in dichloromethane (10 ml) were added. The resulting solution was stirred at room temperature overnight. The reaction was monitored by TLC. The insoluble solids were removed by filtration and the solvent was evaporated. The remnant was dissolved in ethyl acetate, and then washed with 2% NaHCO₃ solution, distilled water, brine, and dried over Na₂SO₄. After the solvent was removed under reduced pressure, the crude product was purified by flash column chromatography to yield 129 as a pale yellow oil in 90% yield.

### Step 2.2 Synthesis of [1-(4-chloro-benzoyl)-5-methoxy-2-methyl-1H-indol-3-yl]-acetic acid 4-(2-hydroxy-ethyl)-phenyl ester (130)

Compound 129 (7 mmol) obtained in step 2.1 above was dissolved in THF (40 ml) and reacted with 1 M solution of tetrabutylammonium fluoride (TBAF) in THF (7.2 mmol) and acetic acid (7 ml) at room temperature for 3 h. Alcohol 130 was obtained as a pale yellow solid in 88% yield. MS: 477 (M+).

### Step 2.3 Synthesis of [1-(4-Chloro-benzoyl)-5-methoxy-2-methyl-1H-indol-3-yl]-acetic acid 4-[2-(diethoxy-phosphoryloxy)-ethyl]-phenyl ester (93)

Diethylchlorophosphate (2.5 ml, 17.26 mmol) was added drop-wise to a solution of alcohol 112 (6.64 mmol) in dichloromethane (10 ml) containing DIPEA (2.2 ml, 13.28 mmol), followed by DMAP (25 mg) as a solid. The reaction mixture was heated under reflux overnight. The reaction solution was washed with water (2×25 ml), dried over anhydrous Na₂SO₄, filtered and concentrated. The crude residue was purified by column chromatography using n-hexane:ethyl acetate (40:60) as eluant. The pure fractions were combined and evaporated to give 93 as viscous yellowish oil in 82% yield. MS: 613.16 (M+).

Toxicity Assessment of phospho-tyrosol-indomethacin 93

The cardiotoxicity of phospho-tyrosol-indomethacin 93 (PTI) was determined histologically in heart tissue sections from mice treated with PTI 93 for about 1.5 months There were no differences between PTI-treated and healthy control mice. Its genotoxicity was evaluated by measuring the ability of PTI to induce reverse mutations of two strains of Salmonella Typhimurium (TA98 and TA100) in the presence and absence of rat liver S9 activation. These studies showed no genotoxicity.

Pharmacokinetics

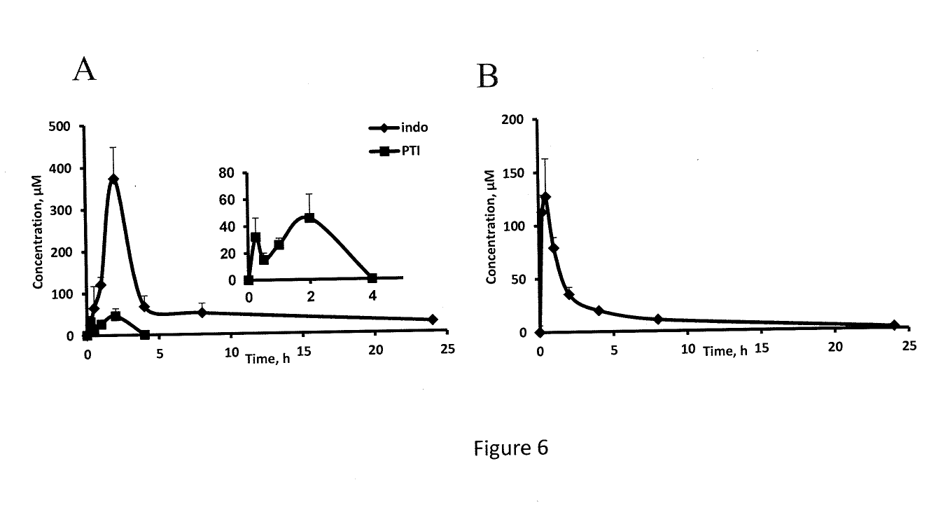

FIG. 6 illustrates a pharmacokinetics study of PTI 93 in mice. Following a single i.p. dose of 100 mg/kg PTI 93 (left) or 58 mg/kg indomethacin (127) (equimolar to PTI) (Indo; right) the plasma levels of intact PTI 93 and indomethacin (127) (hydrolysis product of PTI) were determined at the indicated time points.

Plasma levels of PTI 93 reached the maximum concentration (Cₘₐₓ=46 μM) at 2 hours and became undetectable 4 hours post administration. Its metabolite, indomethacin, reached its Cₘₐₓ=378 μM at 2.5 hours and was minimal at 24 hours. Conventional indomethacin 127 (given alone as above) Cₘₐₓ=127 μM at 1 hour, with its levels becoming negligible at 24 hours. PTI generated a cumulative AUC₀₋₂₄ₕ (PTI plus indomethacin) of 1,700 pMxh, while that of indomethacin was 500 pMxh. This result was totally unexpected.

Efficacy of PTI 93 Against Lung Cancer

Methods:

A549 human non-small cell lung cancer cells (1.5×10⁵) were injected subcutaneously in the left and right flanks of 5-6-week-old female NOD SCID mice (Taconic Farms, Germantown, N.Y.). When the average tumor volume reached 100 mm³, mice were treated by oral gavage of 10 or 15 mg/kg/day PTI 93 or vehicle (corn oil) for 2 weeks, when they were sacrificed and the tumors were harvested.

Results;

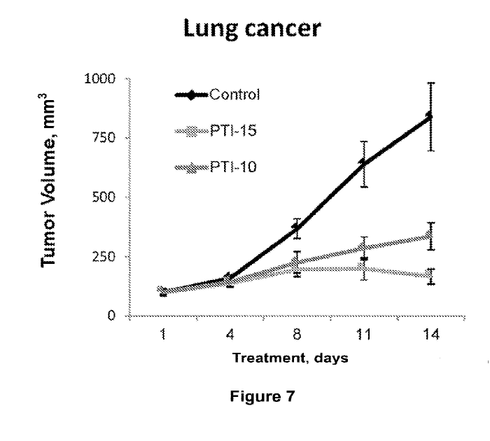

As shown in FIG. 7 (values are Mean±SEM), at the end of the study at 10 mg/kg/day PTI 93 reduced tumor volume by 85%, and at 15 mg/kg/day by 96% (factoring in the 0 time value). These changes are significant (p<0.001, compared to control).

### Example 3

### Aerosol Administration of Phospho-Sulindac (PS, 96) Prevents Non-Small Cell Lung Cancer

Inhalation Exposure System:

Air flow in the system was controlled by two major devices by using the arrangement illustrated by FIG. 1: (1) an inlet air regulator which pushes air into the system via the baffle; and (2) a vacuum pump which draws air from the system.

PS 96 was dissolved in ethanol. PS solution in the baffle was aerosolized with the ultrasonic atomizer. The aerosol passed through an ascending stainless steel column, followed by a reflux column which is maintained at a temperature gradient by a heating tape (82° C.) and a chiller (5° C.) to condense and remove ethanol. PS aerosol exiting the reflux column then passed through a charcoal column which served to remove residual traces of ethanol from aerosol before it entered the animal-holding chamber. Experimental animals were held in nose-only air-tight tubes for designated time intervals.

Orthotopic Lung Cancer Model:

BALB/c nude mice (7 weeks old) were divided into control and treatment groups (15 mice/group) and treated following a prevention protocol by administration of either aerosol generated from ethanol (control) or PS solution (treatment) for one week. The optimized exposure time and dose to mice were 50 mg/mL PS for 8 min, respectively. On day 1 of week 2, a small incision (˜5 mm) was made to the left side of the chest of anesthetized mice and 1 million GFP-A549 human lung cancer cells (A549 cells expressing green fluorescence protein (GFP) which allows their detection and quantification) were injected into their left lung as described by Y. Doki et al. (Br. J. Cancer, 79, 7-8, pages 1121-1126, 1999). Inhalation treatment was resumed 2 days post-surgery and continued for 6 weeks when mice were euthanized, and blood and lung tissues were collected. Luminosity of the GFP-A549 tumors was measured and the lungs were weighed.

Chemopreventive Efficacy:

Two outcomes were used to gauge efficacy, animal survival and tumor size.

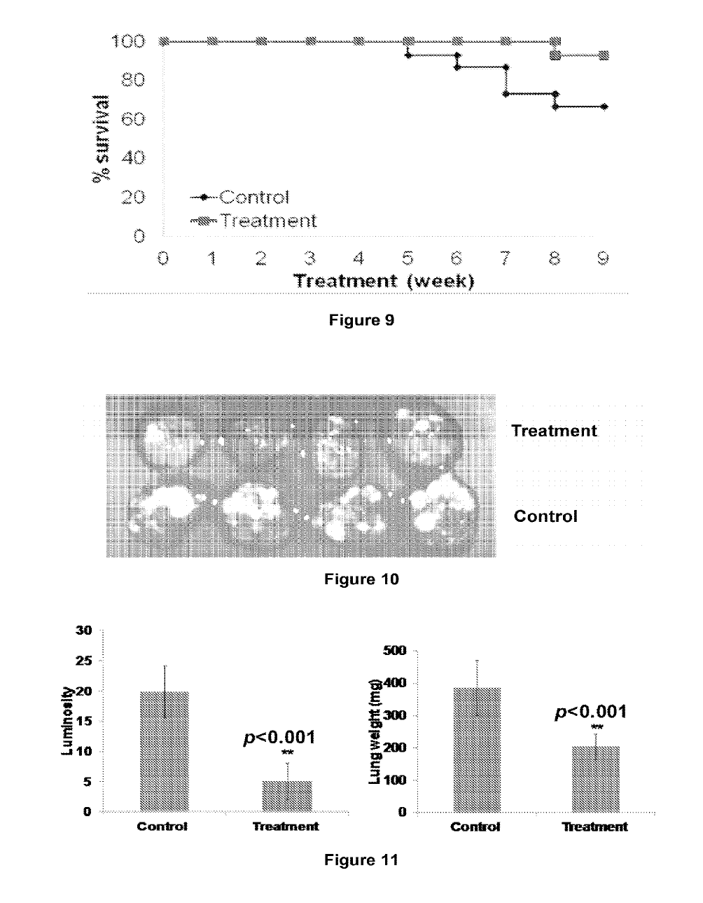

a) Survival: At the end of the study, 40% of the mice in the control group died from the disease while the death rate in the treatment group was less than 10% (p<0.03). The results are illustrated by FIG. 9.

b) Tumor size: At sacrifice, the tumor size was (all values, Mean±SEM) determined a) by luminosity: control=19.85±4.33, treatment=5.05±2.97 (p<0.001). The results are shown in FIG. 10 (upper photograph: after treatment; lower photograph: control group) and FIG. 11 (left hand side); and b) by lung weight: control=385.7±85.2 mg, treatment=204.4±39.4 mg (p<0.001). The results are shown in FIG. 11 (right hand side).

### Example 4

### The Pharmacokinetic Parameters of PS 96 after Inhalational Administration

PS 96 as well as sulindac, sulindac sulfide 131 and sulindac sulfone 132, the structures of which are shown below, were administered to BALB/c nude mice.

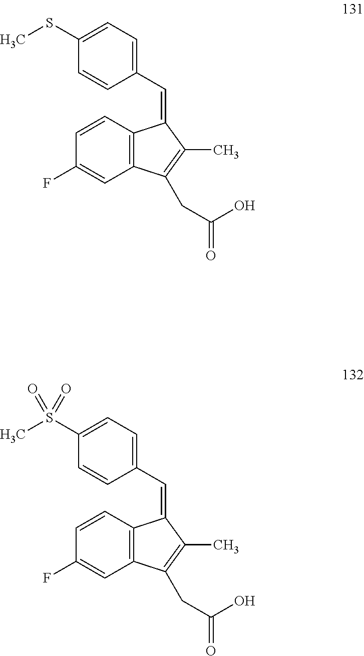 [SMILES: CSc1ccc(/C=C2/C(C)=C(CC(=O)O)c3cc(F)ccc32)cc1 ; CC1=C(CC(=O)O)c2cc(F)ccc2/C1=C\c1ccc(S(C)(=O)=O)cc1]

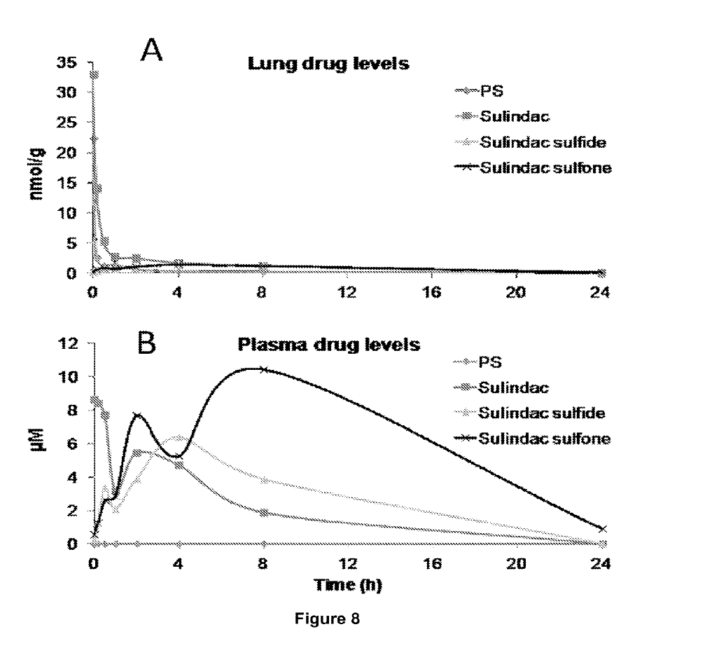

After 8 min of inhalation treatment, BALB/c nude mice were euthanized at various time points and drug levels were analyzed by HPLC in plasma and lung tissues. The results are summarized below and are further illustrated in FIG. 8.

TABLE 1
Pharmacokinetic parameters in lung

| | AUC | Cₘₐₓ, nmol/g | Tₘₐₓ, h |
|---|---|---|---|
| PS 96 | 7.7 | 22.2 | 0 |
| Sulindac | 30.1 | 32.9 | 0 |
| Sulindac sulfide 131 | 18.9 | 1.4 | 4 |
| Sulindac sulfone 132 | 57.5 | 4.6 | 8 |

TABLE 2
Pharmacokinetic parameters in plasma

| | AUC | Cₘₐₓ, μM | Tₘₐₓ, h |
|---|---|---|---|
| PS 96 | 0 | 0 | — |
| Sulindac | 49.5 | 8.6 | 0 |
| Sulindac sulfide 131 | 66.9 | 6.4 | 4 |
| Sulindac sulfone 132 | 142.4 | 10.4 | 8 |

These findings indicate the following: a) inhalation provides intact PS 96 to the lungs, which is more cytotoxic to human cancer cells than either of its three metabolites, sulindac, sulindac sulfide 131 and sulindac sulfone 132; b) oral administration does not provide intact PS 96 to the lungs, leading only to its three metabolites; and c) there are sufficient concentrations of sulindac and its metabolites in the circulation, and for prolonged periods of time. Sulindac, sulindac sulfide 131 and sulindac sulfone 132 are established cancer chemopreventive agents and thus, when derived from inhaled PS 96, they can prevent smoking/nicotine-related cancers at sites other than the lung.

### Example 5

### Inhalation Delivery of Aerosolized Phospho-Sulindac to the Lungs of Mice Leads to Higher Drug Levels than Oral Administration

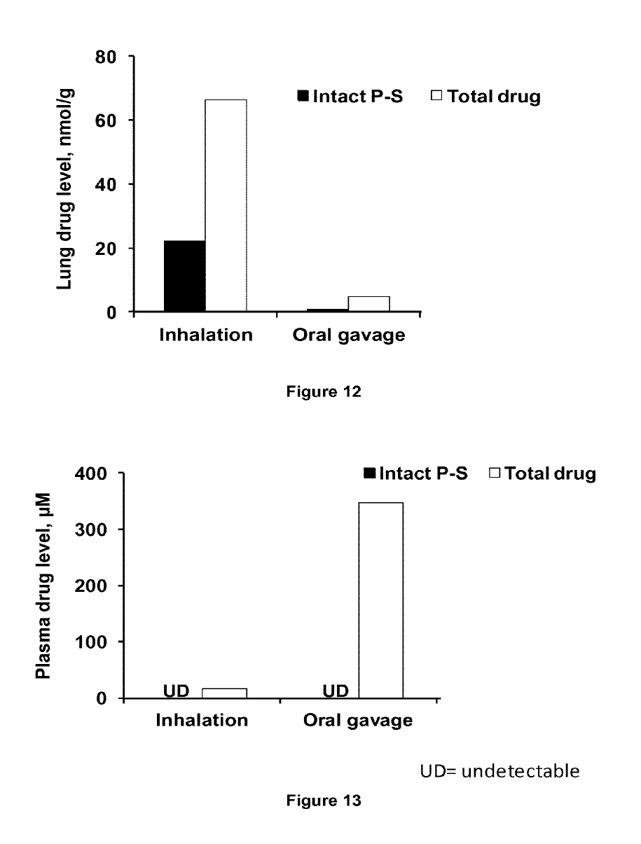

The delivery of aerosolized phospho-sulindac (PS 96) to the lungs of mice was evaluated using the same inhalation device as in Example 1 and compared to its oral delivery. The PS 96 doses were: inhalational=6.5 mg/kg body weight; oral=150 mg/kg body weight. The level of PS 96 in the lungs and plasma after inhalation vs. after oral gavage are shown in FIGS. 12 and 13, respectively.

Lungs:

PS levels: The aerosol-exposure system delivered a high level of intact PS 96 to the lungs of mice (>20 nmol/g); while there were only trace levels of intact PS 96 (<2 nmol/g) by oral administration.

Total Drug Levels:

It represents the total level of PS 96 plus its metabolites. The main metabolites of PS 96 are sulindac, sulindac sulfide 131 and sulindac sulfone 132; at least the first two can cause gastrointestinal and renal side effects. The levels achieved by inhalation were significantly higher compared to those by oral administration.

Plasma:

PS levels: undetectable.

Total drug levels after inhalation treatment (17 μM) was lower than that after oral (348 μM) administration. Thus, inhalation delivery leads to blood levels of sulindac that can be chemopreventive for various non-lung cancers, but which are not particularly high so that can have significant potential toxicity. Of the three main metabolites of PS 96, at least sulindac and sulindac sulfide 131 can cause gastrointestinal and renal side effects.

Thus, PS 96 can be effectively delivered to lung cells by inhalation of a mixture of tobacco smoke with aerosolized PS 96.

### Example 6

### Inhibition of Glioblastoma Cell Lines

U87 cells were treated with sulindac and ibuprofen 125 as well as with the compounds 2, 3, 4 and 96. U87 is a human primary glioblastoma cell line, formally known as U-87 MG. This cell line has epithelial morphology, and is one of the most frequently used glioblastoma cell lines. In this experiment the 24-hour growth inhibitory concentration (24-h IC₅₀) of phospho-sulindac, phospho-ibuprofen, phospho-ibuprofen glycerol, and phospho-ibuprofen glycerol amide were determined, as specified by Huang et al. (Huang L, Mackenzie G G. Sun Y, Ouyang N, Xie G, Vrankova K. et al. Cancer Res. 2011; 71: pp. 7617-27).

The values of 24-h IC₅₀, μM are summarized in Table 3 below.

TABLE 3

| | 24-h IC₅₀, μM |
|---|---|
| Sulindac | ≧1000 |
| Ibuprofen 125 | ≧1000 |
| Phospho-sulindac 96 | 114 |
| Phospho-ibuprofen 2 | 98 |
| Phospho-ibuprofen glycerol 3 | 105 |
| Phospho-ibuprofen glycerol amide 4 | 87 |

Thus, the compounds of the present invention 2, 3, 4 and 96 inhibited glioblastoma cell lines U87 with enhanced potency compared to conventional NSAIDs sulindac and ibuprofen 125.

### Example 7

### Phosphovalproic Acid (PV, 116) and Ibuprofen Phospho-Gylcerol Amide (PGIA, 4) Synergize Strongly to Inhibit the Growth of Glioblastoma and Lung Cancer

Methods:

Cell Growth:

After treatment with PV 116 or PGIA 4 alone or in combination for 24 h, the reduction of 3-(4,5-dimethylthiazol-2-yl)-2,5-diphenyltetrazolium bromide dye (MTT), was determined following the manufacturer's protocol (Promega, Madison, Wis.).

### Apoptosis:

Cells (1.0×10⁵ cells/well) were treated with or without PV 116, PGIA 4 or valproic acid (VPA) for 24 or 48 h. After treatment, cells were trypsinized, stained with Annexin V-FITC (100× dilution, Invitrogen) and propidium iodide (PI) 0.5 μg/ml and the fluorescence intensity was analyzed by FACScaliber.

Results:

PGIA 4 is a Successful Combination Partner with PV 116 in Inhibiting Glioblastoma Cell Growth In Vitro

The potential synergy of PV and PGIA was screened in vitro. Isobologram established synergy between PV and PGIA.

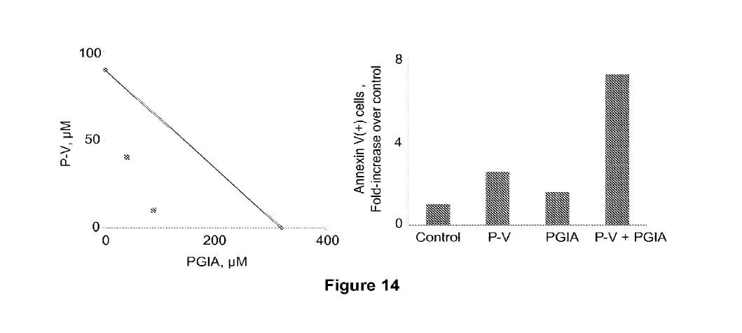

It was observed that in cultured U87 glioblastoma cells, there is a clear-cut pharmacological synergy between PV and PGIA (FIG. 14, left panel). In addition, there is also a synergistic effect in the induction of apoptosis. For example, after 24 h of incubation with PGIA 200 μM and PV 40 μM, the fold-increase of annexinV (+) cells was 8.0, compared to 3.0 for PV 40 μM alone and 1.8 for PGIA 200 μM (FIG. 14, right panel).

Similar results were obtained in other glioblastoma cell lines, such as U118. LN-18 and LN-229, as well as in A549 lung cancer and MIA PaCa-2 pancreatic cancer cell lines.

1. A method of treating and/or preventing lung or brain cancer and/or precancerous conditions thereof, comprising administering a compound of Formula I 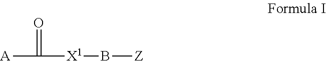
or an enantiomer, diastereomer, racemate, tautomer, salt or hydrate thereof, wherein:
A is an optionally substituted aliphatic, heteroaliphatic, aromatic, heteroaromatic substituent or alkylaryl substituent having 1 to 100 carbon atoms;
X¹ is selected from the group consisting of —O—, —S— and —NR¹—,
R¹ being hydrogen or C₁₋₁₀₀-alkyl;
B is selected from 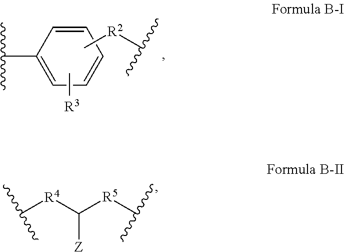
a single bond and an aliphatic group with 1 to 100 carbon atoms,
R², R⁴ and R⁵ being the same or different C₁₋₃-alkylene,
R₃ being hydrogen, C₁₋₆-alkyl, halogenated C₁₋₆-alkyl; C₁₋₆-alkoxy, halogenated C₁₋₆-alkoxy, —C(O)—C₁₋₆-alkyl, —C(O)O—C₁₋₆-alkyl, —OC(O)—C-₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxyl;
Z is selected from 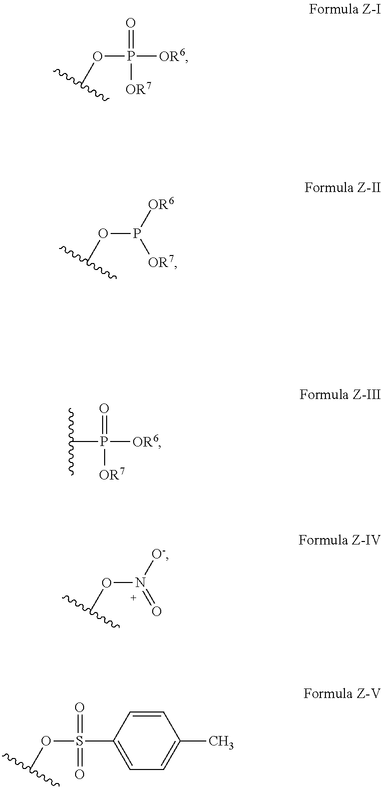
and a folic acid residue;
R⁶ being independently selected from hydrogen, C₁₋₁₀₀-alkyl, and a polyethylene glycol residue,
R⁷ being independently selected from hydrogen, C₁₋₁₀₀-alkyl, and a polyethylene glycol residue; or
B together with Z forms a structure: 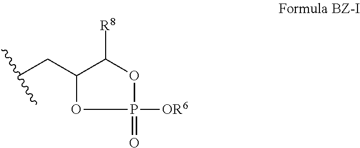
R⁶ being defined as above, and
R⁸ being independently selected from hydrogen, an aliphatic substituent with 1 to 22 carbon atoms, and a polyethylene glycol residue;
to a human or animal by the respiratory route.

2. The method according to claim 1, wherein A is selected from 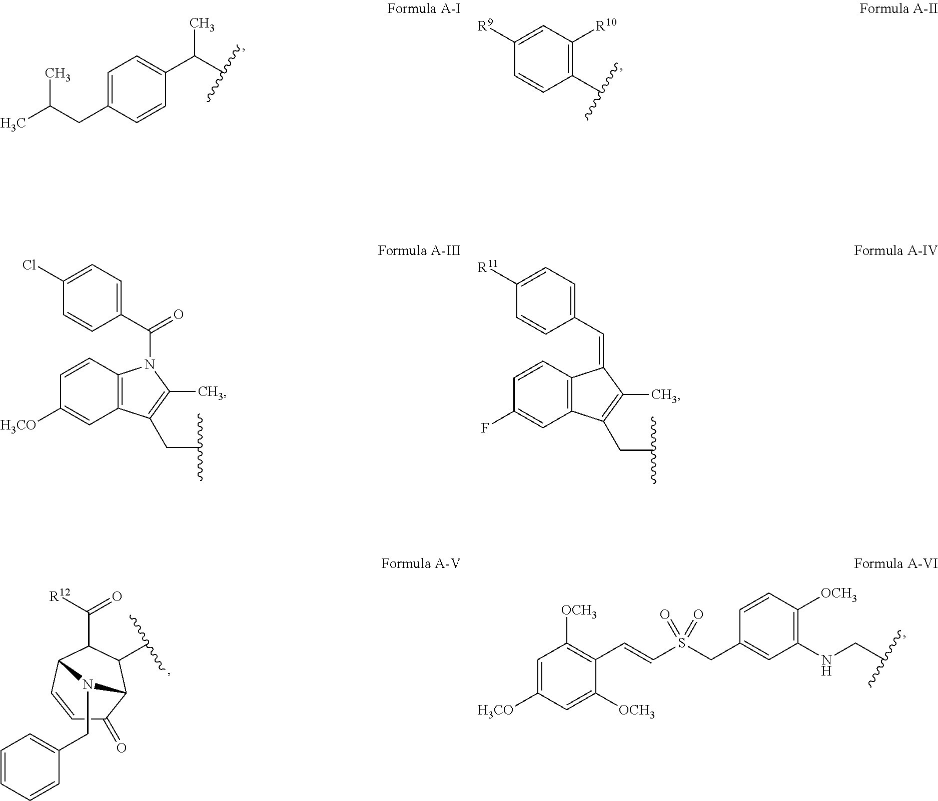 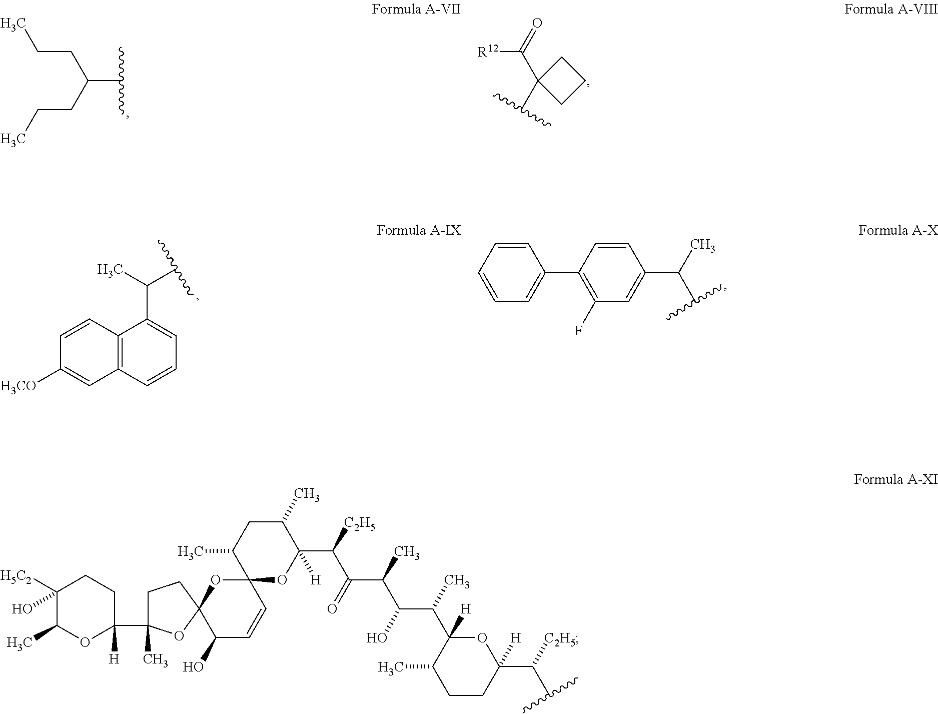 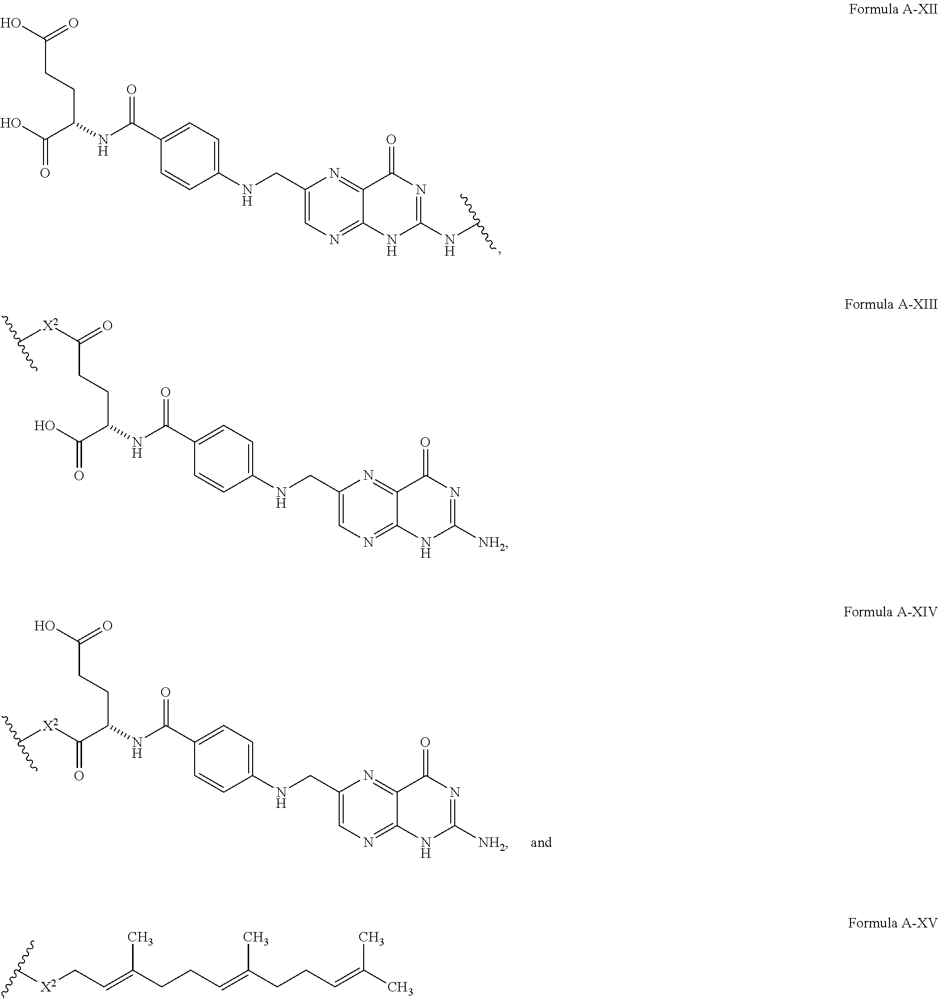
R⁹ being selected from hydrogen and trifluoromethyl;
R¹⁰ being selected from —X₂—C(O)—CH₃,
R¹¹ being selected from —SCH₃, —S(O)CH₃ and —S(O)₂CH₃; and
R¹² being selected from hydroxy, —B—Z and Formula A-XII,
wherein X² is selected from the group consisting of —O—, —S— and —NR¹³—,
R¹³ being hydrogen or C₁₋₆-alkyl.

3. The method according to claim 1, wherein the folic acid residue is selected from

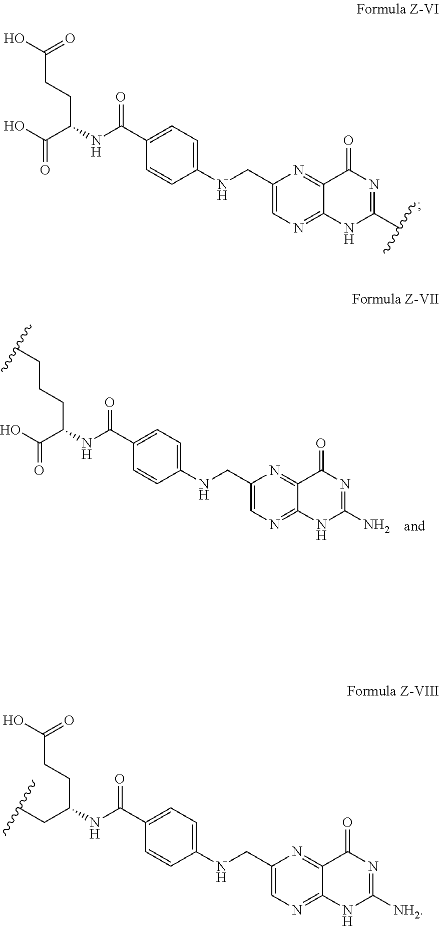 [SMILES: *c1nc(=O)c2nc(CNc3ccc(C(=O)N[C@@H](CCC(=O)O)C(=O)O)cc3)cnc2[nH]1 ; *CCC[C@H](NC(=O)c1ccc(NCc2cnc3[nH]c(N)nc(=O)c3n2)cc1)C(=O)O ; "and" ; *C[C@H](CCC(=O)O)NC(=O)c1ccc(NCc2cnc3[nH]c(N)nc(=O)c3n2)cc1]

4. The method according to claim 1, wherein X¹ is —NR¹—, R¹ is hydrogen;
B is selected from 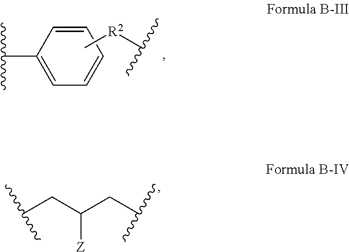
single bond, C₁₋₆-alkylene, C₂₋₆-alkenylene and C₂₋₆-alkynylene;
Z is represented by formula Z-I,
R⁶ being independently selected from hydrogen, C₁₋₃-alkyl and (OCH₂CH₂)_{b}OCH₃, and
R⁷ being independently selected from C₁₋₃-alkyl and (OCH₂CH₂)ₙOCH₃; wherein N is from 40-50.

5. The method according to claim 1, wherein X¹ is —NR¹—, R¹ is hydrogen;
B is selected from the group consisting of C-₁₋₄-alkylene and 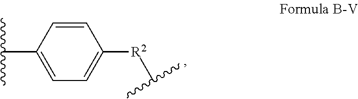
R² being methylene or ethylene; and
Z is represented by Formula Z-I, R⁶ and R⁷ being identical C₁₋₃-alkyl substituents.

6. The method according to claim 1, wherein X¹ is —NR¹—, R¹ is hydrogen; B is —(CH₂)₄—, and Z is represented by Formula Z-I, R⁶ and R⁷ being identical C₁₋₃-alkyl substituents.

7. (canceled)

8. The method according to claim 1 selected from

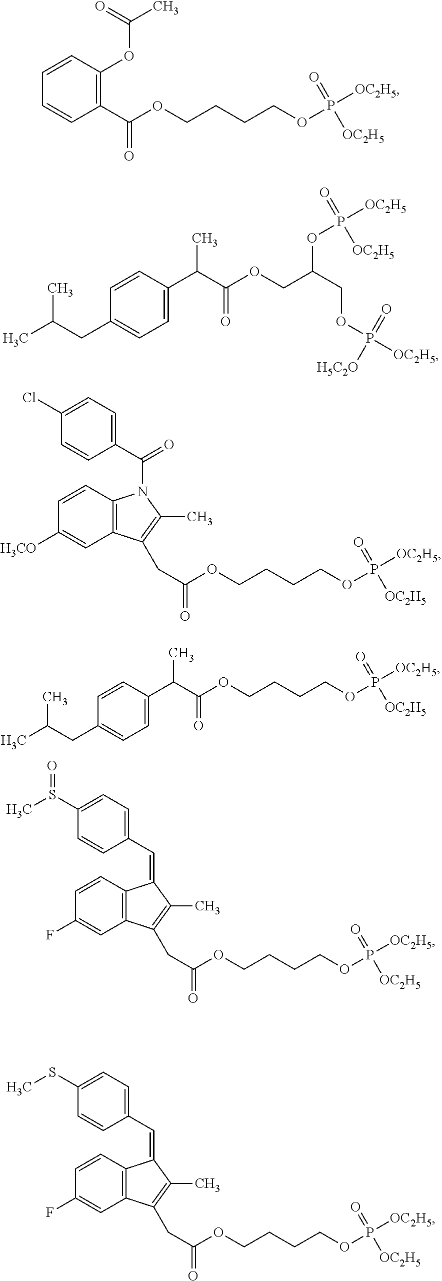

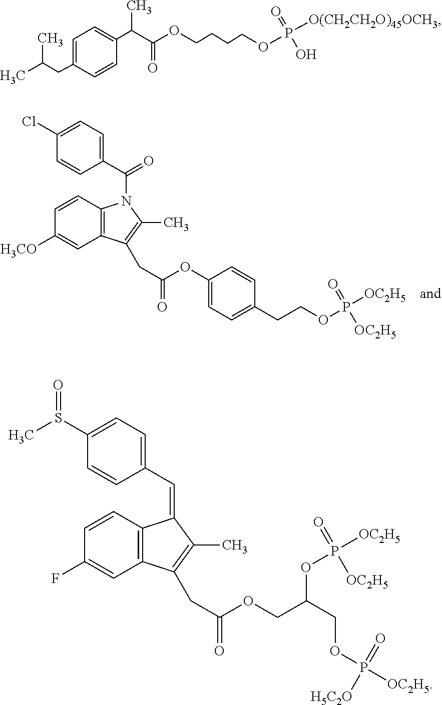

9. The compound according to claim 1 selected from

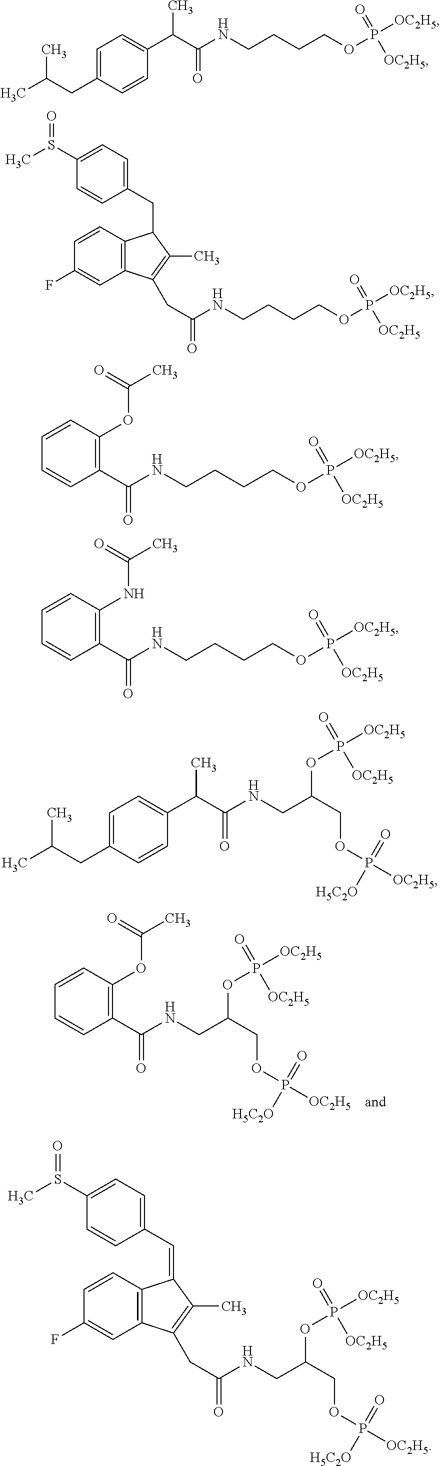

10. The method according to claim 1, wherein said method is to prevent a precancerous lung lesion.

11. The method according to any one of claim 1, wherein said method is to prevent a precancerous brain lesion.

12. The method according to claim 1, wherein said method is to prevent or to treat lung cancer a precancerous lung lesion.

13. The method according to claim 1, wherein said method is to prevent or to treat brain cancer.

14. The method according to claim 1, wherein said method is to treat small cell or non-small cell lung cancer.

15. The method according to claim 13, wherein the brain cancer is glioma.

16. The method according to claim 1, wherein the compound is administered as a pharmaceutical composition comprising the compound and a pharmaceutically acceptable excipient.

17. The method pharmaceutical composition according to claim 16, comprising administering said composition to a human or animal by nasal administration.

18. The method according to claim 16, comprising administering said composition to a human or animal in the form of an aerosol.

19. The method according to claim 16, comprising administering said composition to a human or animal in the form of a dry powder aerosol.

20. The method according to claim 16, wherein said composition is formulated in form of nanoparticles.

21. The method of claim 20, wherein said nanoparticles are lipid or polymeric nanoparticles or a combination thereof.

22. The method of claim 20, wherein said nanoparticles are in form of a liposome, submicron emulsion, microemulsion, nanoemulsion, lipid micelle, solid lipid nanoparticle, polymeric micelle, polymeric nanoparticle or a combination thereof.

23. The method according to claim 16, further comprising administering one or more additional compounds having anticancer activity.

24. The method according to claim 23, wherein the one or more additional compounds having anticancer activity comprise difluoromethylornithine, erlotinib, imatinib, or thiostrepton.

25. The method according to claim 16, wherein said composition is administered to a human or animal in combination with tobacco or tobacco smoke.

26. The method according to claim 16, wherein the compound is administered via an inhalation device comprising the pharmaceutical composition.

27. The method according to claim 16, wherein the compound is administered via a smoking device comprising tobacco and the pharmaceutical composition.

28. The method according to claim 27, wherein said smoking device is a cigarette.

29. The method according to claim 27, wherein the pharmaceutical composition is spatially separated from the tobacco.

30-47. (canceled)
